SEQUENCE LISTING (360 sequences)

SEQ ID NO: 1 - protein, 123 aa, synthetic construct
  REGION 1..123: note = VH A23-58
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSS

SEQ ID NO: 2 - protein, 8 aa, synthetic construct
  REGION 1..8: note = HCDR1 A23-58
GFTFTSSA

SEQ ID NO: 3 - protein, 8 aa, synthetic construct
  REGION 1..8: note = HCDR2 A23-58
IVVGSGNT

SEQ ID NO: 4 - protein, 17 aa, synthetic construct
  REGION 1..17: note = HCDR3 A23-58
AAPNCSNVVCYDGFDIW

SEQ ID NO: 5 - protein, 108 aa, synthetic construct
  REGION 1..108: note = VL A23-58
EIVLTQSPGTLSLSPGERATLSCRASQSVSSSYLAWYQQKPGQAPRLLIYSASSRATGIPDRFSGSGSGTDFTLTISRLEPEDFAVYFCQQYGTSPWTFGQGTKVEIK

SEQ ID NO: 6 - protein, 7 aa, synthetic construct
  REGION 1..7: note = LCDR1 A23-58
QSVSSSY

SEQ ID NO: 7 - skipped

SEQ ID NO: 8 - protein, 10 aa, synthetic construct
  REGION 1..10: note = LCDR3 A23-58
QQYGTSPWTF

SEQ ID NO: 9 - protein, 126 aa, synthetic construct
  REGION 1..126: note = VH A19-46
QVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSS

SEQ ID NO: 10 - protein, 8 aa, synthetic construct
  REGION 1..8: note = HCDR1 A19-46
GFTLSSYG

SEQ ID NO: 11 - protein, 8 aa, synthetic construct
  REGION 1..8: note = HCDR2 A19-46
ISYDGSNK

SEQ ID NO: 12 - protein, 19 aa, synthetic construct
  REGION 1..19: note = HCDR3 A19-46
ARGWAYWELLPDYYYGMDV

SEQ ID NO: 13 - protein, 110 aa, synthetic construct
  REGION 1..110: note = VL A19-46
QTVVTQEPSFSVSPGGTVTLTCGLSSGSVSTAYFPSWYQQTPGQAPRTLIYGTNTRSSGVPDRFSGSILGNKAALTITGAQADDESDYYCVLYMGRGIVVFGGGTKLTVL

SEQ ID NO: 14 - protein, 9 aa, synthetic construct
  REGION 1..9: note = LCDR1 A19-46
SGSVSTAYF

SEQ ID NO: 15 - skipped

SEQ ID NO: 16 - protein, 10 aa, synthetic construct
  REGION 1..10: note = LCDR3 A19-46
VLYMGRGIVV

SEQ ID NO: 17 - protein, 123 aa, synthetic construct
  REGION 1..123: note = VH B1-182
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSS

SEQ ID NO: 18 - protein, 8 aa, synthetic construct
  REGION 1..8: note = HCDR1 B1-182
GFTFTSSA

SEQ ID NO: 19 - protein, 8 aa, synthetic construct
  REGION 1..8: note = HCDR2 B1-182
IVVGSGNT

SEQ ID NO: 20 - protein, 17 aa, synthetic construct
  REGION 1..17: note = HCDR3 B1-182
AAPYCSGGSCFDGFDIW

SEQ ID NO: 21 - protein, 108 aa, synthetic construct
  REGION 1..108: note = VL B1-182
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIR

SEQ ID NO: 22 - protein, 7 aa, synthetic construct
  REGION 1..7: note = LCDR1 B1-182
QSVSSSY

SEQ ID NO: 23 - skipped

SEQ ID NO: 24 - protein, 10 aa, synthetic construct
  REGION 1..10: note = LCDR3 B1-182
QQYGNSPWTF

SEQ ID NO: 25 - protein, 127 aa, synthetic construct
  REGION 1..127: note = VH A19-61
QVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSS

SEQ ID NO: 26 - protein, 8 aa, synthetic construct
  REGION 1..8: note = HCDR1 A19-61
GFTFSSYA

SEQ ID NO: 27 - protein, 8 aa, synthetic construct
  REGION 1..8: note = HCDR2 A19-61
ISYDGSNQ

SEQ ID NO: 28 - protein, 22 aa, synthetic construct
  REGION 1..22: note = HCDR3 A19-61
CARDLAIAVAGTWHYYNGMDVW

SEQ ID NO: 29 - protein, 107 aa, synthetic construct
  REGION 1..107: note = VL A19-61
DIQMTQSPSSVSASVGDRVIITCRASQGISSWLAWYQQKPGKAPKVLIYDASSLQSGVPSRFSGSGYGTDFTLTISSLQPEDSATYYCQQAKSFPITFGQGTRLEIK

SEQ ID NO: 30 - protein, 6 aa, synthetic construct
  REGION 1..6: note = LCDR1 A19-61
QGISSW

SEQ ID NO: 31 - skipped

SEQ ID NO: 32 - protein, 10 aa, synthetic construct
  REGION 1..10: note = LCDR3 A19-61
QQAKSFPITF

SEQ ID NO: 33 - protein, 129 aa, synthetic construct
  REGION 1..129: note = VH A23-58H/B1-182L
SASGSGQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSS

SEQ ID NO: 34 - protein, 8 aa, synthetic construct
  REGION 1..8: note = HCDR1 A23-58H/B1-182L
GFTFTSSA

SEQ ID NO: 35 - protein, 8 aa, synthetic construct
  REGION 1..8: note = HCDR2 A23-58H/B1-182L
IVVGSGNT

SEQ ID NO: 36 - protein, 17 aa, synthetic construct
  REGION 1..17: note = HCDR3 A23-58H/B1-182L
AAPNCSNVVCYDGFDIW

SEQ ID NO: 37 - protein, 108 aa, synthetic construct; identical to SEQ ID NO: 21
  REGION 1..108: note = VL A23-58H/B1-182L

SEQ ID NO: 38 - protein, 7 aa, synthetic construct
  REGION 1..7: note = LCDR1 A23-58H/B1-182L
QSVSSSY

SEQ ID NO: 39 - skipped

SEQ ID NO: 40 - protein, 10 aa, synthetic construct
  REGION 1..10: note = LCDR3 A23-58H/B1-182L
QQYGNSPWTF

SEQ ID NO: 41 - protein, 19 aa, synthetic construct
  REGION 1..19: note = signal peptide
MGWSCIILFLVATATGVHS

SEQ ID NO: 42 - protein, 103 aa, synthetic construct
  REGION 1..103: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 43 - protein, 107 aa, synthetic construct [sequence omitted]

SEQ ID NO: 44 - protein, 226 aa, synthetic construct
  REGION 1..226: note = Fc - 1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 45 - protein, 226 aa, synthetic construct
  REGION 1..226: note = Fc - 2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 46 - protein, 5 aa, synthetic construct
  REGION 1..5: note = Linker
DKTHT

SEQ ID NO: 47-50 - skipped (4 entries)

SEQ ID NO: 51 - protein, 4 aa, synthetic construct
  REGION 1..4: note = Linker
SGSG

SEQ ID NO: 52 - protein, 5 aa, synthetic construct
  REGION 1..5: note = Linker
GGSSG

SEQ ID NO: 53 - protein, 5 aa, synthetic construct
  REGION 1..5: note = Linker
GGSGS

SEQ ID NO: 54 - protein, 5 aa, synthetic construct
  REGION 1..5: note = Linker
GSSGS

SEQ ID NO: 55 - protein, 5 aa, synthetic construct
  REGION 1..5: note = Linker
GTVAA

SEQ ID NO: 56 - protein, 5 aa, synthetic construct
  REGION 1..5: note = Linker
ASGGS

SEQ ID NO: 57 - protein, 5 aa, synthetic construct
  REGION 1..5: note = Linker
ASTGG

SEQ ID NO: 58 - protein, 6 aa, synthetic construct
  REGION 1..6: note = Linker
ASGGSG

SEQ ID NO: 59 - protein, 7 aa, synthetic construct
  REGION 1..7: note = Linker
GGSGSSG

SEQ ID NO: 60 - protein, 10 aa, synthetic construct
  REGION 1..10: note = Linker
GGSGGSSGSS

SEQ ID NO: 61 - protein, 10 aa, synthetic construct
  REGION 1..10: note = Linker
SGGSGSSGGS

SEQ ID NO: 62 - protein, 7 aa, synthetic construct
  REGION 1..7: note = Linker
SGSGGSG

SEQ ID NO: 63 - protein, 9 aa, synthetic construct
  REGION 1..9: note = Linker
SGSGGSSGG

SEQ ID NO: 64 - protein, 11 aa, synthetic construct
  REGION 1..11: note = Linker
SGSGGSSGGSG

SEQ ID NO: 65 - protein, 13 aa, synthetic construct
  REGION 1..13: note = Linker
GSGSGGSSGGSSG

SEQ ID NO: 66 - protein, 10 aa, synthetic construct
  REGION 1..10: note = Linker
GGSGSGGGSG

SEQ ID NO: 67 - protein, 11 aa, synthetic construct
  REGION 1..11: note = Linker
GGSGSGGGSGG

SEQ ID NO: 68 - protein, 15 aa, synthetic construct
  REGION 1..15: note = Linker
GGSSGSGSGGSGSSG

SEQ ID NO: 69 - protein, 14 aa, synthetic construct
  REGION 1..14: note = Linker
GGSGGSGGGSSGSG

SEQ ID NO: 70 - protein, 17 aa, synthetic construct
  REGION 1..17: note = Linker
GGSGGSGSGGGSASGSG

SEQ ID NO: 71 - protein, 18 aa, synthetic construct
  REGION 1..18: note = Linker
GGSGGSGSGGGGSASGSG

SEQ ID NO: 72 - protein, 20 aa, synthetic construct
  REGION 1..20: note = Linker
GGGSSGGGGSGGSGSGGSGS

SEQ ID NO: 73 - protein, 20 aa, synthetic construct
  REGION 1..20: note = Linker
GGGGSGGSGSGGGGSASGSG

SEQ ID NO: 74 - protein, 20 aa, synthetic construct
  REGION 1..20: note = Linker
GGGSSGGSGSGGSGSGGSGS

SEQ ID NO: 75 - protein, 22 aa, synthetic construct
  REGION 1..22: note = Linker
SGGSSGGSGSGGSGSGGSGSSG

SEQ ID NO: 76 - protein, 22 aa, synthetic construct
  REGION 1..22: note = Linker
GSGSSGGGGSGGSGSGGSGSSG

SEQ ID NO: 77 - protein, 40 aa, synthetic construct
  REGION 1..40: note = Linker
GGGGSGSGGSGGGSSGGGGSGGGGSGGGGSGGGGSGGGGS

SEQ ID NO: 78 - protein, 40 aa, synthetic construct
  REGION 1..40: note = Linker
GGGGSGGGGSGGGGSGGGGSGGGGSGGGGSGGGGSGGGGS

SEQ ID NO: 79 - protein, 43 aa, synthetic construct
  REGION 1..43: note = Linker
GGGGSGSGGSGGGSSGGGGSGGGGSGGGGSGGGGSGGGGSSSS

SEQ ID NO: 80 - protein, 45 aa, synthetic construct
  REGION 1..45: note = Linker
GGGGSGSGGSGGGSSGGGGSGGGGSGGGGSGGGGSGGGGSSSSGS

SEQ ID NO: 81 - protein, 588 aa, synthetic construct
  REGION 1..588: note = A19-46_A23-58__CODVH_holeLS - peptide without signal
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTQVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 82 - protein, 607 aa, synthetic construct
  REGION 1..607: note = A19-46_A23-58__CODVH_holeLS - peptide with signal
MGWSCIILFLVATATGVHSQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTQVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 83 - protein, 588 aa, synthetic construct
  REGION 1..588: note = A19-46_B1-182 CODVH holeLS - peptide without signal
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTQVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 84 - protein, 607 aa, synthetic construct
  REGION 1..607: note = A19-46_B1-182 CODVH holeLS - peptide with signal
MGWSCIILFLVATATGVHSQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTQVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 85 - protein, 335 aa, synthetic construct
  REGION 1..335: note = A19-46_B1-182 CODVL - peptide without signal
QTVVTQEPSFSVSPGGTVTLTCGLSSGSVSTAYFPSWYQQTPGQAPRTLIYGTNTRSSGVPDRFSGSILGNKAALTITGAQADDESDYYCVLYMGRGIVVFGGGTKLTVLDKTHTEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 354 aa, synthetic construct
  REGION 1..354: note = A19-46_B1-182 CODVL - peptide with signal
MGWSCIILFLVATATGVHSQTVVTQEPSFSVSPGGTVTLTCGLSSGSVSTAYFPSWYQQTPGQAPRTLIYGTNTRSSGVPDRFSGSILGNKAALTITGAQADDESDYYCVLYMGRGIVVFGGGTKLTVLDKTHTEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 455 aa, synthetic construct
  REGION 1..455: note = A19-46 monoH knobLS - peptide without signal
QVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 88 - protein, 474 aa, synthetic construct
  REGION 1..474: note = A19-46 monoH knobLS - peptide with signal
MGWSCIILFLVATATGVHSQVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 89 - protein, 217 aa, synthetic construct
  REGION 1..217: note = A19-46 monoL - peptide without signal
QTVVTQEPSFSVSPGGTVTLTCGLSSGSVSTAYFPSWYQQTPGQAPRTLIYGTNTRSSGVPDRFSGSILGNKAALTITGAQADDESDYYCVLYMGRGIVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 236 aa, synthetic construct
  REGION 1..236: note = A19-46 monoL - peptide with signal
MGWSCIILFLVATATGVHSQTVVTQEPSFSVSPGGTVTLTCGLSSGSVSTAYFPSWYQQTPGQAPRTLIYGTNTRSSGVPDRFSGSILGNKAALTITGAQADDESDYYCVLYMGRGIVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 589 aa, synthetic construct
  REGION 1..589: note = A19-61_A23-58 CODVH hole LS - peptide without signal
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTQVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 92 - protein, 608 aa, synthetic construct
  REGION 1..608: note = A19-61_A23-58 CODVH hole LS - peptide with signal
MGWSCIILFLVATATGVHSQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTQVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 93 - protein, 589 aa, synthetic construct
  REGION 1..589: note = A19-61_B1-182 CODVH holeLS - peptide without signal
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTQVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 94 - protein, 608 aa, synthetic construct
  REGION 1..608: note = A19-61_B1-182 CODVH holeLS - peptide with signal
MGWSCIILFLVATATGVHSQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTQVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 95 - protein, 332 aa, synthetic construct
  REGION 1..332: note = A19-61_B1-182 CODVL - peptide without signal
DIQMTQSPSSVSASVGDRVIITCRASQGISSWLAWYQQKPGKAPKVLIYDASSLQSGVPSRFSGSGYGTDFTLTISSLQPEDSATYYCQQAKSFPITFGQGTRLEIKDKTHTEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 351 aa, synthetic construct
  REGION 1..351: note = A19-61_B1-182 CODVL - peptide with signal
MGWSCIILFLVATATGVHSDIQMTQSPSSVSASVGDRVIITCRASQGISSWLAWYQQKPGKAPKVLIYDASSLQSGVPSRFSGSGYGTDFTLTISSLQPEDSATYYCQQAKSFPITFGQGTRLEIKDKTHTEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 456 aa, synthetic construct
  REGION 1..456: note = A19-61 monoH knobLS - peptide without signal
QVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 98 - protein, 475 aa, synthetic construct
  REGION 1..475: note = A19-61 monoH knobLS - peptide with signal
MGWSCIILFLVATATGVHSQVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 99 - protein, 214 aa, synthetic construct
  REGION 1..214: note = A19-61 monoL - peptide without signal
DIQMTQSPSSVSASVGDRVIITCRASQGISSWLAWYQQKPGKAPKVLIYDASSLQSGVPSRFSGSGYGTDFTLTISSLQPEDSATYYCQQAKSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 233 aa, synthetic construct
  REGION 1..233: note = A19-61 monoL - peptide with signal
MGWSCIILFLVATATGVHSDIQMTQSPSSVSASVGDRVIITCRASQGISSWLAWYQQKPGKAPKVLIYDASSLQSGVPSRFSGSGYGTDFTLTISSLQPEDSATYYCQQAKSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 588 aa, synthetic construct
  REGION 1..588: note = A23-58_A19-46 CODVH hole LS - peptide without signal
QVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 102 - protein, 607 aa, synthetic construct
  REGION 1..607: note = A23-58_A19-46 CODVH hole LS - peptide with signal
MGWSCIILFLVATATGVHSQVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 103 - protein, 584 aa, synthetic construct
  REGION 1..584: note = A23-58_A19-61__CODVH_holeLS - peptide without signal
QVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 104 - protein, 603 aa, synthetic construct
  REGION 1..603: note = A23-58_A19-61__CODVH_holeLS - peptide with signal
MGWSCIILFLVATATGVHSQVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 105 - protein, 585 aa, synthetic construct
  REGION 1..585: note = A23-58_A23-58 CODVH hole LS - peptide without signal
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 106 - protein, 604 aa, synthetic construct
  REGION 1..604: note = A23-58_A23-58 CODVH hole LS - peptide with signal
MGWSCIILFLVATATGVHSQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 107 - protein, 452 aa, synthetic construct
  REGION 1..452: note = A23-58 monoH KnobLS - peptide without signal
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 108 - protein, 471 aa, synthetic construct
  REGION 1..471: note = A23-58 monoH KnobLS - peptide with signal
MGWSCIILFLVATATGVHSQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 109 - protein, 588 aa, synthetic construct
  REGION 1..588: note = B1-182_A19-46 CODVH holeLS - peptide without signal
QVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 110 - protein, 607 aa, synthetic construct
  REGION 1..607: note = B1-182_A19-46 CODVH holeLS - peptide with signal
MGWSCIILFLVATATGVHSQVQLVESGGGVVQPGRSLRLSCAASGFTLSSYGMHWVRQAPGKGLEWVAVISYDGSNKYYVDSVKGRFTISRDNSKNTLYLQMNSLRAEDTAVYYCARGWAYWELLPDYYYGMDVWGQGTTVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 111 - protein, 335 aa, synthetic construct
  REGION 1..335: note = B1-182_A19-46 CODVL - peptide without signal
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTQTVVTQEPSFSVSPGGTVTLTCGLSSGSVSTAYFPSWYQQTPGQAPRTLIYGTNTRSSGVPDRFSGSILGNKAALTITGAQADDESDYYCVLYMGRGIVVFGGGTKLTVLDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 354 aa, synthetic construct
  REGION 1..354: note = B1-182_A19-46 CODVL - peptide with signal
MGWSCIILFLVATATGVHSEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTQTVVTQEPSFSVSPGGTVTLTCGLSSGSVSTAYFPSWYQQTPGQAPRTLIYGTNTRSSGVPDRFSGSILGNKAALTITGAQADDESDYYCVLYMGRGIVVFGGGTKLTVLDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 332 aa, synthetic construct
  REGION 1..332: note = B1-182_A19-61 CODVL - peptide without signal
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTDIQMTQSPSSVSASVGDRVIITCRASQGISSWLAWYQQKPGKAPKVLIYDASSLQSGVPSRFSGSGYGTDFTLTISSLQPEDSATYYCQQAKSFPITFGQGTRLEIKDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 351 aa, synthetic construct
  REGION 1..351: note = B1-182_A19-61 CODVL - peptide with signal
MGWSCIILFLVATATGVHSEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTDIQMTQSPSSVSASVGDRVIITCRASQGISSWLAWYQQKPGKAPKVLIYDASSLQSGVPSRFSGSGYGTDFTLTISSLQPEDSATYYCQQAKSFPITFGQGTRLEIKDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 585 aa, synthetic construct
  REGION 1..585: note = B1-182_B1-182 CODVH holeLS - peptide without signal
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 116 - protein, 604 aa, synthetic construct
  REGION 1..604: note = B1-182_B1-182 CODVH holeLS - peptide with signal
MGWSCIILFLVATATGVHSQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 117 - protein, 333 aa, synthetic construct
  REGION 1..333: note = B1-182_B1-182 CODVL - peptide without signal
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 352 aa, synthetic construct
  REGION 1..352: note = B1-182_B1-182 CODVL - peptide with signal
MGWSCIILFLVATATGVHSEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 452 aa, synthetic construct
  REGION 1..452: note = B1-182_monoH_knobLS - peptide without signal
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 120 - protein, 471 aa, synthetic construct
  REGION 1..471: note = B1-182_monoH_knobLS - peptide with signal
MGWSCIILFLVATATGVHSQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 121 - protein, 214 aa, synthetic construct
  REGION 1..214: note = B1-182_monoL - peptide without signal
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 233 aa, synthetic construct
  REGION 1..233: note = B1-182_monoL - peptide with signal
MGWSCIILFLVATATGVHSEIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 589 aa, synthetic construct
  REGION 1..589: note = B1-182 A19-61 CODVH holeLS - peptide without signal
QVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 124 - protein, 608 aa, synthetic construct
  REGION 1..608: note = B1-182 A19-61 CODVH holeLS - peptide with signal
MGWSCIILFLVATATGVHSQVQLVESGGGVVQPGRSLRLSCAASGFTFSSYAFHWVRQAPGKGLEWVAVISYDGSNQYYADSVKGRFTISRDNSKNTLYLQMNSLRADDTAVYYCARDLAIAVAGTWHYYNGMDVWGQGTTVTVSSDKTHTQMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSSDKTHTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPG

SEQ ID NO: 125 - DNA, 1824 bp, synthetic construct
  misc_feature 1..1824: note = A19-46_A23-58__CODVH_holeLS - DNA
  source 1..1824: mol_type = other DNA
atgggctggtcctgcatcatcctgtttctggtggccacagccaccggtgtccacagccagatgcagctggtgcagtccggccctgaggtgaaaaagcccggcacctctgtgaaggtctcctgcaaggccagcggctttaccttcacaagcagcgccgtgcagtgggtgcggcaggccagaggccaaagactggaatggatcggctggatcgtggtgggcagcggaaataccaactacgcccagaagttccaggagcgggtgacaatcaccagagatatgagcacatctaccgcttatatggaactgagctctctgagaagcgaggacaccgccgtgtactactgcgccgctcctaactgcagcaacgtggtttgttacgacggcttcgacatctggggccagggaacaatggtcaccgtgtcctctgataagacccacacacaggtccagctggtggaaagcggaggcggagttgttcagcctggcagaagcctgagactgagctgtgccgcctctggctttaccctgagcagctatggcatgcactgggttcgacaagcccctggcaaaggcctggaatgggttgccgtgatcagctacgacggcagcaacaagtactacgtggactccgtgaagggcagattcaccatcagccgggacaacagcaagaacaccctgtacctgcagatgaactccctgagagccgaagatacagccgtgtattactgtgccagaggctgggcctattgggagctgctgcccgattactactacggcatggatgtgtggggacagggaaccaccgtgaccgtgtctagcgacaagacacacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 126 - DNA, 1824 bp, synthetic construct
  misc_feature 1..1824: note = A19-46_B1-182 CODVH holeLS - DNA
  source 1..1824: mol_type = other DNA
atgggctggtcctgcatcatcctgtttctggtggccacagccaccggtgtccacagccagatgcagctggtgcagtctggccccgaagtgaagaaacctggcaccagcgtgaaggtgtcctgcaaggccagcggcttcacctttacaagctctgccgtgcagtgggtccgacaggctagaggacaacggctggaatggatcggctggatcgttgtcggcagcggcaacaccaattacgcccagaaattccaagagcgcgtgaccatcaccagagacatgagcaccagcaccgcctacatggaactgagcagcctgagaagcgaggacaccgccgtgtactattgcgccgctccttactgtagcggcggcagctgctttgacggctttgacatttggggccagggcaccatggtcaccgtgtcctctgataagacccacacacaggtccagctggtggaaagcggaggcggagttgttcagcctggcagaagcctgagactgagctgtgccgcctctggctttaccctgagcagctatggcatgcactgggttcgacaagcccctggcaaaggcctggaatgggttgccgtgatcagctacgacggcagcaacaagtactacgtggactccgtgaagggcagattcaccatcagccgggacaacagcaagaacaccctgtacctgcagatgaactccctgagagccgaagatacagccgtgtattactgtgccagaggctgggcctattgggagctgctgcccgattactactacggcatggatgtgtggggacagggaaccaccgtgaccgtgtctagcgacaagacacacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 127 - DNA, 1065 bp, synthetic construct
  misc_feature 1..1065: note = A19-46_B1-182 CODVL - DNA
  source 1..1065: mol_type = other DNA
atgggctggtcctgcatcatcctgtttctggtggccacagccaccggtgtccacagccagaccgtggtcacacaagagcctagcttctccgtgtctcctggcggcacagtgacactgacatgtggccttagcagcggcagcgtgtccaccgcctattttcccagctggtatcagcagacccctggacaggctccccggacactgatctacggcaccaataccagatccagcggcgtgcccgatagattcagcggctctatcctgggcaacaaggccgcactgacaatcacaggcgcccaggccgatgacgagagcgattactactgcgtgctgtacatgggcagaggcatcgtggtttttggcggaggcaccaagctgaccgtgctggataagacccacaccgagatcgtgctgacacagagccctggcacactgtcactgtctccaggcgaaagagccacactgagctgtagagccagccagagcgtgtcctctagctacctggcctggtatcaacaaaagcccggacaggcccctcggctgctgatatatggtgcctctagcagagccaccggctttcccgacagattttctggcagcggaagcggcaccgacttcaccctgacaatctccagactggaacccgaggacttcgccgtgtactactgtcagcagtacggcaacagcccctggacctttggccagggcacaaaggtggaaatccgggacaagacacacacccgtacggtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttga

SEQ ID NO: 128 - DNA, 1425 bp, synthetic construct
  misc_feature 1..1425: note = A19-46 monoH knobLS - DNA
  source 1..1425: mol_type = other DNA
atgggctggtcctgcatcatcctgtttctggtggccacagccaccggtgtccacagccaggtgcagctggttgaatctggtggcggagttgtgcagcctggcagaagcctgagactgtcttgtgccgccagcggctttaccctgagcagctatggaatgcactgggtccgacaggcccctggcaaaggacttgaatgggtcgccgtgatcagctacgacggcagcaacaagtactacgtggacagcgtgaagggcagattcaccatcagccgggacaacagcaagaacaccctgtacctgcagatgaacagcctgagagccgaggacaccgccgtgtactattgtgccagaggctgggcctattgggagctgctgcccgattactactacggcatggatgtgtggggccagggcaccacagtgacagtctcttctgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgctgcatgaagccctgcacagccactacacccagaagtccctgtctctgtctcctggctga

SEQ ID NO: 129 - DNA, 711 bp, synthetic construct
  misc_feature 1..711: note = A19-46 monoL - DNA
  source 1..711: mol_type = other DNA
atgggctggtcctgcatcatcctgtttctggtggccacagccaccggtgtccacagccagaccgtggtcacacaagagcctagcttctccgtgtctcctggcggcacagtgacactgacatgtggccttagcagcggcagcgtgtccaccgcctattttcccagctggtatcagcagacccctggacaggctccccggacactgatctacggcaccaataccagatccagcggcgtgcccgatagattcagcggctctatcctgggcaacaaggccgcactgacaatcacaggcgcccaggccgatgacgagagcgattactactgcgtgctgtacatgggcagaggcatcgtggtttttggcggaggcaccaagctgaccgttctgcgtacggtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttga

SEQ ID NO: 130 - DNA, 1827 bp, synthetic construct
  misc_feature 1..1827: note = A19-61_A23-58 CODVH hole LS - DNA
  source 1..1827: mol_type = other DNA
atgggctggtcctgcatcatcctgtttctggtggccacagccaccggtgtccacagccagatgcagctggtgcagtccggccctgaggtgaaaaagcccggcacctctgtgaaggtctcctgcaaggccagcggctttaccttcacaagcagcgccgtgcagtgggtgcggcaggccagaggccaaagactggaatggatcggctggatcgtggtgggcagcggaaataccaactacgcccagaagttccaggagcgggtgacaatcaccagagatatgagcacatctaccgcttatatggaactgagctctctgagaagcgaggacaccgccgtgtactactgcgccgctcctaactgcagcaacgtggtttgttacgacggcttcgacatctggggccagggaacaatggtcaccgtgtcctctgataagacccacacacaggtccagctggtggaaagcggaggcggagttgttcagcctggcagaagcctgagactgagctgtgccgcctctggctttaccttcagcagctacgccttccactgggttcgacaagcccctggcaaaggcctggaatgggttgccgtgatcagctacgacggcagcaaccagtactacgccgactccgtgaagggcagattcaccatcagccgggacaacagcaagaacaccctgtacctgcagatgaacagcctgagggccgatgatacagccgtgtattactgtgcccgcgacctggccattgccgtggctggaacatggcactactacaacggcatggacgtgtggggacagggaaccaccgtgaccgtgtctagcgacaagacacacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 131 - DNA, 1827 bp, synthetic construct
  misc_feature 1..1827: note = A19-61_B1-182 CODVH holeLS - DNA
  source 1..1827: mol_type = other DNA
atgggctggtcctgcatcatcctgtttctggtggccacagccaccggtgtccacagccagatgcagctggtgcagtctggccccgaagtgaagaaacctggcaccagcgtgaaggtgtcctgcaaggccagcggcttcacctttacaagctctgccgtgcagtgggtccgacaggctagaggacaacggctggaatggatcggctggatcgttgtcggcagcggcaacaccaattacgcccagaaattccaagagcgcgtgaccatcaccagagacatgagcaccagcaccgcctacatggaactgagcagcctgagaagcgaggacaccgccgtgtactattgcgccgctccttactgtagcggcggcagctgctttgacggctttgacatttggggccagggcaccatggtcaccgtgtcctctgataagacccacacacaggtccagctggtggaaagcggaggcggagttgttcagcctggcagaagcctgagactgagctgtgccgcctctggctttaccttcagcagctacgccttccactgggttcgacaagcccctggcaaaggcctggaatgggttgccgtgatcagctacgacggcagcaaccagtactacgccgactccgtgaagggcagattcaccatcagccgggacaacagcaagaacaccctgtacctgcagatgaacagcctgagggccgatgatacagccgtgtattactgtgcccgcgacctggccattgccgtggctggaacatggcactactacaacggcatggacgtgtggggacagggaaccaccgtgaccgtgtctagcgacaagacacacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 132 - DNA, 1056 bp, synthetic construct
  misc_feature 1..1056: note = A19-61_B1-182 CODVL - DNA
  source 1..1056: mol_type = other DNA
atgggctggtcctgcatcatcctgtttctggtggccacagccaccggtgtccacagcgacatccagatgacacagagccctagcagcgtgtccgcctctgtgggagacagagtgatcatcacctgtagagccagccagggcatcagctcttggctggcttggtatcagcagaagcctggcaaggcccctaaggtgctgatctacgatgccagctctctgcagtctggcgtgcccagcagattttctggcagcggctacggcaccgacttcaccctgaccatatctagcctgcagcctgaggacagcgccacctactattgtcagcaggccaagtctttccccatcaccttcggccagggcaccagactggaaatcaaggacaagacccacaccgagatcgtgctgacccagtctcctggcacactgtcactgtctccaggcgagagagccacactgagctgtagagcttctcagagcgtgtccagcagctacctcgcctggtatcaacaaaagcccggacaggctccccggctgctgatatatggtgcctctagcagagccaccggctttcccgatagattcagcggctctggctctggcacagatttcacactgacaatcagcaggctggaacccgaggacttcgccgtgtattactgccagcagtacggcaacagcccctggacatttggccagggaacaaaggtggaaatccgcgataagacacacacccgtacggtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttga

SEQ ID NO: 133 - DNA, 1428 bp, synthetic construct
  misc_feature 1..1428: note = A19-61 monoH knobLS - DNA
  source 1..1428: mol_type = other DNA
atgggctggtcctgcatcatcctgtttctggtggccacagccaccggtgtccacagccaggtgcagctggttgaatctggtggcggagttgtgcagcctggcagaagcctgagactgtcttgtgccgccagcggcttcacctttagcagctacgccttccactgggtccgacaggcacctggcaaaggccttgaatgggtcgccgtgatcagctacgacggcagcaatcagtactacgccgacagcgtgaagggcagattcaccatcagccgggacaacagcaagaacaccctgtacctgcagatgaacagcctgagagccgacgacaccgccgtgtactattgtgcccgcgatctggccattgccgtggccggaacatggcactactacaacggcatggatgtgtggggccagggcaccacagtgacagtctcttctgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgctgcatgaagccctgcacagccactacacccagaagtccctgtctctgtctcctggctga

SEQ ID NO: 134 - DNA, 702 bp, synthetic construct
  misc_feature 1..702: note = A19-61 monoL - DNA
  source 1..702: mol_type = other DNA
atgggctggtcctgcatcatcctgtttctggtggccacagccaccggtgtccacagcgacatccagatgacacagagccctagcagcgtgtccgcctctgtgggagacagagtgatcatcacctgtagagccagccagggcatcagctcttggctggcttggtatcagcagaagcctggcaaggcccctaaggtgctgatctacgatgccagctctctgcagtctggcgtgcccagcagattttctggcagcggctacggcaccgacttcaccctgaccatatctagcctgcagcctgaggacagcgccacctactattgtcagcaggccaagtctttccccatcaccttcggccagggcaccagactggaaatcaagcgtacggtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttga

SEQ ID NO: 135 - DNA, 1824 bp, synthetic construct
  misc_feature 1..1824: note = A23-58_A19-46 CODVH hole LS - DNA
  source 1..1824: mol_type = other DNA
atgggctggtcctgcatcatcctgtttctggtggccacagccaccggtgtccacagccaggtgcagctggttgaatctggtggcggagttgtgcagcctggcagaagcctgagactgtcttgtgccgccagcggctttaccctgagcagctatggaatgcactgggtccgacaggcccctggcaaaggacttgaatgggtcgccgtgatcagctacgacggcagcaacaagtactacgtggacagcgtgaagggcagattcaccatcagccgggacaacagcaagaacaccctgtacctgcagatgaacagcctgagagccgaggacaccgccgtgtactattgtgccagaggctgggcctattgggagctgctgcccgattactactacggcatggatgtgtggggccagggcaccacagtgaccgtgtctagcgataagacccacacacaaatgcagctggtgcagagcggccctgaggtgaaaaagcccggcacatctgtgaaggtgtcctgcaaggccagcggatttaccttcaccagctccgccgtgcagtgggtgcggcaggccagaggccagcggctggaatggatcggatggatcgtggtgggcagcggcaacaccaactacgcccagaagttccaggagagagtgaccatcacaagagatatgagcacaagcactgcttatatggaactgagcagcctgagaagcgaggacaccgccgtgtactactgcgccgctcctaattgcagcaacgtggtttgttacgacggcttcgacatctggggccagggcaccatggtcaccgtgtcttctgacaagacacacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 136 - DNA, 1812 bp, synthetic construct
  misc_feature 1..1812: note = A23-58_A19-61__CODVH_holeLS - DNA
  source 1..1812: mol_type = other DNA
atgggctggtcctgcatcatcctgtttctggtggccacagccaccggtgtccacagccaggtgcagctggttgaatctggtggcggagttgtgcagcctggcagaagcctgagactgtcttgtgccgccagcggcttcacctttagcagctacgccttccactgggtccgacaggcacctggcaaaggccttgaatgggtcgccgtgatcagctacgacggcagcaatcagtactacgccgacagcgtgaagggcagattcaccatcagccgggacaacagcaagaacaccctgtacctgcagatgaacagcctgagagccgacgacaccgccgtgtactattgtgcccgcgatctggccattgccgtggccggaacatggcactactacaacggcatggatgtgtggggccagggcaccacagtgaccgtgtctagcgataagacccacacacaaatgcagctggtgcagagcggccctgaggtgaaaaagcccggcacatctgtgaaggtgtcctgcaaggccagcggatttaccttcaccagctccgccgtgcagtgggtgcggcaggccagaggccagcggctggaatggatcggatggatcgtggtgggcagcggcaacaccaactacgcccagaagttccaggagagagtgaccatcacaagagatatgagcacaagcactgcttatatggaactgagcagcctgagaagcgaggacaccgccgtgtactactgcgccgctcctaattgcagcaacgtggtttgttacgacggcttcgacatctggggccagggcaccatggtcaccgtgtcttctgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 137 - DNA, 1815 bp, synthetic construct
  misc_feature 1..1815: note = A23-58_A23-58 CODVH hole LS - DNA
  source 1..1815: mol_type = other DNA
atgggctggtcctgcatcatcctgtttctggtggccacagccaccggtgtccacagccaaatgcagctggtgcagagcggccctgaggtgaaaaagcccggcacatctgtgaaggtgtcctgcaaggccagcggatttaccttcaccagctccgccgtgcagtgggtgcggcaggccagaggccagcggctggaatggatcggatggatcgtggtgggcagcggcaacaccaactacgcccagaagttccaggagagagtgaccatcacaagagatatgagcacaagcactgcttatatggaactgagcagcctgagaagcgaggacaccgccgtgtactactgcgccgctcctaattgcagcaacgtggtttgttacgacggcttcgacatctggggccagggcaccatggtcaccgtgtcttctgataagacccacacccagatgcagctggtgcagagcggacctgaggtgaaaaagcccggcaccagcgtgaaggtgtcttgcaaggccagcggcttcaccttcacatctagcgccgtgcagtgggtcagacaggccagaggccaaagactggaatggatcggatggatcgtggtgggcagcggcaacaccaactacgcccagaagttccaggagcgggtgaccatcacccgggacatgagcacctctacagcttatatggaactgagcagcctgagaagcgaggacaccgccgtgtactactgcgccgctcctaattgcagcaacgtggtttgttacgacggctttgatatctggggccagggcacaatggtgacagtgtcctccgacaagacccacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 138 - DNA, 1416 bp, synthetic construct
  misc_feature 1..1416: note = A23-58 monoH KnobLS - DNA
  source 1..1416: mol_type = other DNA
atgggctggtcctgcatcatcctgtttctggtggccacagccaccggtgtccacagccagatgcagctggtgcagtccggccctgaggtgaaaaagcccggcacctctgtgaaggtctcctgcaaggccagcggctttaccttcacaagcagcgccgtgcagtgggtgcggcaggccagaggccaaagactggaatggatcggctggatcgtggtgggcagcggaaataccaactacgcccagaagttccaggagcgggtgacaatcaccagagatatgagcacatctaccgcttatatggaactgagctctctgagaagcgaggacaccgccgtgtactactgcgccgctcctaactgcagcaacgtggtttgttacgacggcttcgacatctggggccagggaacaatggtcaccgttagctctgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgctgcatgaagccctgcacagccactacacccagaagtccctgtctctgtctcctggctga

SEQ ID NO: 139 - DNA, 1824 bp, synthetic construct
  misc_feature 1..1824: note = B1-182_A19-46 CODVH holeLS - DNA
  source 1..1824: mol_type = other DNA
atgggctggtcctgcatcatcctgtttctggtggccacagccaccggtgtccacagccaggtgcagctggttgaatctggtggcggagttgtgcagcctggcagaagcctgagactgtcttgtgccgccagcggctttaccctgagcagctatggaatgcactgggtccgacaggcccctggcaaaggacttgaatgggtcgccgtgatcagctacgacggcagcaacaagtactacgtggacagcgtgaagggcagattcaccatcagccgggacaacagcaagaacaccctgtacctgcagatgaacagcctgagagccgaggacaccgccgtgtactattgtgccagaggctgggcctattgggagctgctgcccgattactactacggcatggatgtgtggggccagggcaccacagtgaccgtgtctagcgataagacccacacacagatgcagctcgtgcagagcggccctgaagtgaagaaacctggcacctccgtgaaggtgtcctgcaaggcctctggcttcacctttacaagcagcgccgtgcagtgggttcgacaagctagaggccagagactggaatggatcggctggatcgttgtcggcagcggcaacaccaattacgcccagaaattccaagagcgcgtgaccatcaccagagacatgagcaccagcaccgcctacatggaactgtctagcctgcggagcgaggatacagctgtgtactactgcgccgctccttactgtagcggcggcagctgctttgacggctttgacatttggggacagggaaccatggtcaccgtgtcctctgacaagacacacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 140 - DNA, 1065 bp, synthetic construct
  misc_feature 1..1065: note = B1-182_A19-46 CODVL - DNA
  source 1..1065: mol_type = other DNA
atgggctggtcctgcatcatcctgtttctggtggccacagccaccggtgtccacagcgagatcgtgctgacacagagccctggcacactgtcactgtctccaggcgaaagagccacactgagctgtagagccagccagagcgtgtccagctcttacctggcttggtatcagcagaagcccggacaggctcccagactgctgatctatggcgcctctagcagagccaccggctttcccgatagattttctggcagcggctccggcaccgacttcaccctgacaatcagcagactggaacccgaggacttcgccgtgtactactgtcagcagtacggcaacagcccctggacatttggccagggcaccaaggtggaaatccgggacaagacccacacacagaccgtggtcacacaagagcccagcttctctgtgtctcctggcggcacagtgaccctgacatgtggacttagcagcggatctgtgtccaccgcctactttcccagctggtatcaacagaccccaggccaggcacctcggacactgatctacggcaccaacacaagatccagcggcgtgcccgataggttcagcggctctatcctgggaaacaaggccgcactgactatcacaggcgcccaggccgatgacgagagcgattactactgcgtgctgtacatgggcagaggcatcgtggtttttggcggaggcaccaagctgaccgtgctggataagacacacacccgtacggtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttga

SEQ ID NO: 141 - DNA, 1056 bp, synthetic construct
  misc_feature 1..1056: note = B1-182_A19-61 CODVL - DNA
  source 1..1056: mol_type = other DNA
atgggctggtcctgcatcatcctgtttctggtggccacagccaccggtgtccacagcgagatcgtgctgacacagagccctggcacactgtcactgtctccaggcgaaagagccacactgagctgtagagccagccagagcgtgtccagctcttacctggcttggtatcagcagaagcccggacaggctcccagactgctgatctatggcgcctctagcagagccaccggctttcccgatagattttctggcagcggctccggcaccgacttcaccctgacaatcagcagactggaacccgaggacttcgccgtgtactactgtcagcagtacggcaacagcccctggacatttggccagggcaccaaggtggaaatccgggacaagacccacaccgacatccagatgacccagtctccaagcagcgtgtccgcctctgtgggcgacagagtgatcatcacatgcagagcctctcagggcatcagctcttggctcgcctggtatcaacaaaagcctggcaaggcccctaaggtcctgatctacgatgccagctctctgcagtctggcgtgcccagcagattttccggctctggctacggcacagatttcacactcaccataagcagcctgcagcctgaggacagcgccacctattattgccagcaggccaagtctttccccatcaccttcggccagggaacaagactggaaatcaaggataagacgcacacacgtacggtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttga

SEQ ID NO: 142 - DNA, 1815 bp, synthetic construct
  misc_feature 1..1815: note = B1-182_B1-182 CODVH holeLS - DNA
  source 1..1815: mol_type = other DNA
atgggctggtcctgcatcatcctgtttctggtggccacagccaccggtgtccacagccagatgcagctggtgcagtctggccccgaagtgaagaaacctggcaccagcgtgaaggtgtcctgcaaggccagcggcttcacctttacaagctctgccgtgcagtgggtccgacaggctagaggacaacggctggaatggatcggctggatcgttgtcggcagcggcaacaccaattacgcccagaaattccaagagcgcgtgaccatcaccagagacatgagcaccagcaccgcctacatggaactgagcagcctgagaagcgaggacaccgccgtgtactattgcgccgctccttactgtagcggcggcagctgctttgacggctttgacatttggggccagggcaccatggtcaccgtgtcctctgataagacccacacccagatgcagctggtgcagtctggccccgaagtgaagaaacctggcaccagcgtgaaggtgtcctgcaaggcttctggcttcaccttcacaagcagcgccgtgcagtgggtccgacaggctagaggacagagactggaatggatcggctggatcgtcgtcggcagcggcaacacaaactacgcccagaaattccaagagcgcgtgaccatcaccagagacatgagcaccagcaccgcctacatggaactgagcagcctgagaagcgaggacaccgccgtgtactactgtgccgctccttactgtagcggcggcagctgtttcgacggctttgacatttggggccagggcaccatggtcaccgtgtctagcgacaagacccacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 143 - DNA, 1059 bp, synthetic construct
  misc_feature 1..1059: note = B1-182_B1-182 CODVL - DNA
  source 1..1059: mol_type = other DNA
atgggctggtcctgcatcatcctgtttctggtggccacagccaccggtgtccacagcgagatcgtgctgacacagagccctggcacactgtcactgtctccaggcgaaagagccacactgagctgtagagccagccagagcgtgtccagctcttacctggcttggtatcagcagaagcccggacaggctcccagactgctgatctatggcgcctctagcagagccaccggctttcccgatagattttctggcagcggctccggcaccgacttcaccctgacaatcagcagactggaacccgaggacttcgccgtgtactactgtcagcagtacggcaacagcccctggacatttggccagggcaccaaggtggaaatccgggacaagacccacaccgagatcgtgctgacccagtctccaggcacactgtctcttagcccaggcgagagagctaccctgtcctgtagagcctctcagtccgtgtcctcctcttacctggcctggtatcagcagaagccaggacaggctcccaggctgctgatctatggcgcttcttccagagccaccggctttcccgatagattctccggctctggatctggcaccgacttcaccctgaccatctccagactggaacccgaggacttcgccgtgtactactgccagcagtacggcaactccccatggaccttcggacagggcaccaaggtggaaatcagggacaagacccacacccgtacggtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgttga

SEQ ID NO: 144 - DNA, 1416 bp, synthetic construct
  misc_feature 1..1416: note = B1-182_monoH_knobLS - DNA
  source 1..1416: mol_type = other DNA
atgggctggtcctgcatcatcctgtttctggtggccacagccaccggtgtccacagccagatgcagctggtgcagtctggccccgaagtgaagaaacctggcaccagcgtgaaggtgtcctgcaaggccagcggcttcacctttacaagctctgccgtgcagtgggtccgacaggctagaggacaacggctggaatggatcggctggatcgttgtcggcagcggcaacaccaattacgcccagaaattccaagagcgcgtgaccatcaccagagacatgagcaccagcaccgcctacatggaactgagcagcctgagaagcgaggacaccgccgtgtactattgcgccgctccttactgtagcggcggcagctgctttgacggctttgacatttggggccagggcaccatggtcaccgttagctctgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacccttcctccatgccgggatgagctgaccaagaaccaggtgtccctgtggtgtctcgtgaagggcttctaccccagcgacattgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgctgcatgaagccctgcacagccactacacccagaagtccctgtctctgtctcctggctga

SEQ ID NO: 145 - DNA, 702 bp, synthetic construct
  misc_feature 1..702: note = B1-182_monoL - DNA
  source 1..702: mol_type = other DNA
tcaacattcgccccggttgaagctcttggtcacagggctagaaaggccctggtgggtcacttcgcaggcgtacactttgtgcttctcgtagtcggccttgctcagtgtcagggtgctgctcaggctgtaggtagagtccttgctgtcctgctcggtcacgctctcttggctattgccgctctgcagggcgttgtccaccttccactgcaccttggcttctctggggtagaagttgttcagcaggcacacgacagaggctgtgccagacttcagctgctcgtcggaaggtgggaagatgaacacgctaggagcggccaccgtacggatttccaccttggtgccctggccaaatgtccaggggctgttgccgtactgctgacagtagtacacggcgaagtcctcgggttccagtctgctgattgtcagggtgaagtcggtgccggagccgctgccagaaaatctatcgggaaagccggtggctctgctagaggcgccatagatcagcagtctgggagcctgtccgggcttctgctgataccaagccaggtaagagctggacacgctctggctggctctacagctcagtgtggctctttcgcctggagacagtgacagtgtgccagggctctgtgtcagcacgatctcgctgtggacaccggtggctgtggccaccagaaacaggatgatgcaggaccagcccat

SEQ ID NO: 146 - DNA, 1827 bp, synthetic construct
  misc_feature 1..1827: note = B1-182 A19-61 CODVH holeLS - DNA
  source 1..1827: mol_type = other DNA
atgggctggtcctgcatcatcctgtttctggtggccacagccaccggtgtccacagccaggtgcagctggttgaatctggtggcggagttgtgcagcctggcagaagcctgagactgtcttgtgccgccagcggcttcacctttagcagctacgccttccactgggtccgacaggcacctggcaaaggccttgaatgggtcgccgtgatcagctacgacggcagcaatcagtactacgccgacagcgtgaagggcagattcaccatcagccgggacaacagcaagaacaccctgtacctgcagatgaacagcctgagagccgacgacaccgccgtgtactattgtgcccgcgatctggccattgccgtggccggaacatggcactactacaacggcatggatgtgtggggccagggcaccacagtgaccgtgtctagcgataagacccacacacagatgcagctcgtgcagagcggccctgaagtgaagaaacctggcacctccgtgaaggtgtcctgcaaggcctccggctttaccttcacaagctctgccgtgcagtgggttcgacaagccagaggccaacggctggaatggatcggctggatcgttgtcggcagcggcaacaccaattacgcccagaaattccaagagcgcgtgaccatcaccagagacatgagcaccagcaccgcctacatggaactgtctagcctgcggagcgaggatacagccgtgtattactgcgccgctccttactgtagcggcggcagctgctttgacggctttgacatttggggacagggaaccatggtcaccgtgtcctctgacaagacacacaccgcctctacaaagggcccctccgtgtttcctctggctcccagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctcgcgaacctcaagtctgtacactgcctcctagccgggacgagctgaccaagaatcaggtgtccctgagctgtgccgtgaagggcttttaccccagcgacattgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgctgcatgaagccctgcacagccactacacccagaagtccctgtctctgtctcctggctga

SEQ ID NO: 147 - protein, 119 aa, synthetic construct
  REGION 1..119: note = LY-Cov1404_VH
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSS

SEQ ID NO: 148 - protein, 9 aa, synthetic construct
  REGION 1..9: note = LY-Cov1404_VH CDR1
GFSLSISGV

SEQ ID NO: 149 - protein, 5 aa, synthetic construct
  REGION 1..5: note = LY-Cov1404_VH CDR2
YWDDD

SEQ ID NO: 150 - protein, 9 aa, synthetic construct
  REGION 1..9: note = LY-Cov1404_VH CDR3
HSISTIFDH

SEQ ID NO: 151 - protein, 109 aa, synthetic construct
  REGION 1..109: note = LY-Cov1404_VL
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVL

SEQ ID NO: 152 - protein, 14 aa, synthetic construct
  REGION 1..14: note = LY-Cov1404_VL CDR1
TATSSDVGDYNYVS

SEQ ID NO: 153 - protein, 7 aa, synthetic construct
  REGION 1..7: note = LY-Cov1404_VL CDR2
EVSDRPS

SEQ ID NO: 154 - protein, 9 aa, synthetic construct
  REGION 1..9: note = LY-Cov1404_VL CDR3
SSYTTSSAV

SEQ ID NO: 155 - protein, 109 aa, synthetic construct
  REGION 1..109: note = VL B8
EIVMTQSPAILSVSPGERATLSCRASQSVAINLAWYQQKPGQAPRLLIYAASSRATGIPGRFSGSGSETNFTLTISSLQSGDFAVYYCQQYITWPPSYTFGQGTKLEIK

SEQ ID NO: 156 - protein, 11 aa, synthetic construct
  REGION 1..11: note = LCDR1 B8
RASQSVAINLA

SEQ ID NO: 157 - protein, 7 aa, synthetic construct
  REGION 1..7: note = LCDR2 B8
AASSRAT

SEQ ID NO: 158 - protein, 11 aa, synthetic construct
  REGION 1..11: note = LCDR3 B8
QQYITWPPSYT

SEQ ID NO: 159 - protein, 117 aa, synthetic construct
  REGION 1..117: note = VH B8
EVQLVESGGALIQPGGSLRLSCAASGIIVSANYMSWVRQAPGKGLEWVSVIYSGGSTFYADSVKGRFTISRDNSKNTLYLEMNSLRAEDTAVYYCARDAPILGVSDWGQGTLVTVSS

SEQ ID NO: 160 - protein, 8 aa, synthetic construct
  REGION 1..8: note = HCDR1 B8
GIIVSANY

SEQ ID NO: 161 - protein, 7 aa, synthetic construct
  REGION 1..7: note = HCDR2 B8
IYSGGST

SEQ ID NO: 162 - protein, 11 aa, synthetic construct
  REGION 1..11: note = HCDR3 B8
ARDAPILGVSD

SEQ ID NO: 163 - protein, 108 aa, synthetic construct
  REGION 1..108: note = VL E12
EIVLTQSPGTLSLSPGETATLSCRASQSFSSDLAWYQQKPGQAPRLLIYGASSRATGIPDRFSGSGSGTDFTLTISRLEPADFAVYYCQQYGSSPPVTFGGGTKVEIK

SEQ ID NO: 164 - protein, 6 aa, synthetic construct
  REGION 1..6: note = LCDR1 E12
QSFSSD

SEQ ID NO: 165 - skipped

SEQ ID NO: 166 - protein, 10 aa, synthetic construct
  REGION 1..10: note = LCDR3 E12
QQYGSSPPVT

SEQ ID NO: 167 - protein, 118 aa, synthetic construct
  REGION 1..118: note = VH E12
EVQLVESGGGLIQPGGSLRLSCAASGVIVSYNYMNWVRQAPGKGLEWVSVIYSGGSTFYADSVRGRFTISRDNSKNTLYLQMNSLRAEDTAVYYCARDLGPAGAFDIWGLGTMVTVSS

SEQ ID NO: 168 - protein, 8 aa, synthetic construct
  REGION 1..8: note = HCDR1 E12
GVIVSYNY

SEQ ID NO: 169 - protein, 7 aa, synthetic construct
  REGION 1..7: note = HCDR2 E12
IYSGGST

SEQ ID NO: 170 - protein, 12 aa, synthetic construct
  REGION 1..12: note = HCDR3 E12
ARDLGPAGAFDI

SEQ ID NO: 171 - protein, 113 aa, synthetic construct
  REGION 1..113: note = VL E8
DTVMTQSPLSLPVTPGEPASISCRSSQSLLNSNGYNYLDWYVQKPGQSPQLLIYLGSNRASGVPDRFSGSGSGTDFTLKISRVEAEDVGVYYCMQPLQTPLFTFGPGTKVEIK

SEQ ID NO: 172 - protein, 11 aa, synthetic construct
  REGION 1..11: note = LCDR1 E8
QSLLNSNGYNY

SEQ ID NO: 173 - skipped

SEQ ID NO: 174 - protein, 10 aa, synthetic construct
  REGION 1..10: note = LCDR3 E8
MQPLQTPLFT

SEQ ID NO: 175 - protein, 125 aa, synthetic construct
  REGION 1..125: note = VH E8
QVTLRESGPALVKPTQTLTLTCTFSGFSLSTRGVCVSWIRQPPGKALEWLAFIDWDDDKYYNTSLKTRLTISKDTSKNQVVLTMTNVHPVDTATYYCARISSHHGSSWCTTFDYWGQGALVTVSA

SEQ ID NO: 176 - protein, 10 aa, synthetic construct
  REGION 1..10: note = HCDR1 E8
GFSLSTRGVC

SEQ ID NO: 177 - protein, 7 aa, synthetic construct
  REGION 1..7: note = HCDR2 E8
IDWDDDK

SEQ ID NO: 178 - protein, 17 aa, synthetic construct
  REGION 1..17: note = HCDR3 E8
ARISSHHGSSWCTTFDY

SEQ ID NO: 179 - protein, 109 aa, synthetic construct
  REGION 1..109: note = VL A23-58H/L93VGLTG
EIVLTQSPGTLSLSPGERATLSCRASQSVSSSYLAWYQQKPGQAPRLLIYSASSRATGIPDRFSGSGSGTDFTLTISRLEPEDFAVYFCQQYVGLTGWTFGQGTKVEIK

SEQ ID NO: 180 - protein, 7 aa, synthetic construct
  REGION 1..7: note = LCDR1 A23-58H/L93VGLTG
QSVSSSY

SEQ ID NO: 181 - protein, 4 aa, synthetic construct
  REGION 1..4: note = LCDR2 A23-58H/L93VGLTG
SASS

SEQ ID NO: 182 - protein, 10 aa, synthetic construct
  REGION 1..10: note = LCDR3 A23-58H/L93VGLTG
QQYVGLTGWT

SEQ ID NO: 183 - protein, 123 aa, synthetic construct; identical to SEQ ID NO: 1
  REGION 1..123: note = VH A23-58H/L93VGLTG

SEQ ID NO: 184 - protein, 8 aa, synthetic construct
  REGION 1..8: note = HCDR1 A23-58H/L93VGLTG
GFTFTSSA

SEQ ID NO: 185 - protein, 8 aa, synthetic construct
  REGION 1..8: note = HCDR2 A23-58H/L93VGLTG
IVVGSGNT

SEQ ID NO: 186 - protein, 16 aa, synthetic construct
  REGION 1..16: note = HCDR3 A23-58H/L93VGLTG
AAPNCSNVVCYDGFDI

SEQ ID NO: 187 - protein, 226 aa, synthetic construct
  REGION 1..226: note = Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 188 - protein, 109 aa, synthetic construct
  REGION 1..109: note = 58.1LC_94GLTG - VL
EIVLTQSPGTLSLSPGERATLSCRASQSVSSSYLAWYQQKPGQAPRLLIYSASSRATGIPDRFSGSGSGTDFTLTISRLEPEDFAVYFCQQYGGLTGWTFGQGTKVEIK

SEQ ID NO: 189 - protein, 109 aa, synthetic construct
  REGION 1..109: note = S2E12LC - VL
DIVLTQTPGTLSLSPGERATLSCRASQSVSSSYLAWYQQKPGQAPRLLIYGASSRATGIPDRFSGSGSGTDFTLTISRLEPEDFAVYYCQQYVGLTGWTFGQGTKVEIK

SEQ ID NO: 190 - protein, 123 aa, synthetic construct
  REGION 1..123: note = A23-58.1HC_Y100cA - VH
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCADGFDIWGQGTMVTVSS

SEQ ID NO: 191 - protein, 109 aa, synthetic construct
  REGION 1..109: note = 182.1LC_94GLTG - VL
EIVLTQSPGTLSLSPGERATLSCRASQSVSSSYLAWYQQKPGQAPRLLIYGASSRATGFPDRFSGSGSGTDFTLTISRLEPEDFAVYYCQQYGGLTGWTFGQGTKVEIR

SEQ ID NO: 192 - protein, 123 aa, synthetic construct
  REGION 1..123: note = A23-105.1 - VH
QVQLVESGGGVVQPGRSLRLSCAASGFTFSNYAIHWVRQAPGKGLEWVAVISYDGSNKYYADSVKGRFTISRDNSKNTLYLEMNSLRAEDMAVYYCARVGPYQYDSSAAFDIWGQGTMVTVSS

SEQ ID NO: 193 - protein, 8 aa, synthetic construct
  REGION 1..8: note = A23-105.1 HCDR1
GFTFSNYA

SEQ ID NO: 194 - protein, 8 aa, synthetic construct
  REGION 1..8: note = A23-105.1 HCDR2
ISYDGSNK

SEQ ID NO: 195 - protein, 16 aa, synthetic construct
  REGION 1..16: note = A23-105.1 HCDR3
CARVGPYQYDSSAAFD

SEQ ID NO: 196 - protein, 107 aa, synthetic construct
  REGION 1..107: note = A23-105.1 VL
DIQMTQSPSTLSASVGDRVTITCRASQSISSWLAWYQQKPGQAPKLLIYDASSLESGVPSRFSGSGSGTEFTLTISSLQPDDFATYYCQQYNSYSRTFGQGTKVEIK

SEQ ID NO: 197 - protein, 6 aa, synthetic construct
  REGION 1..6: note = A23-105.1 LCDR1
QSISSW

SEQ ID NO: 198 - skipped

SEQ ID NO: 199 - protein, 11 aa, synthetic construct [sequence omitted]

SEQ ID NO: 200 - protein, 120 aa, synthetic construct
  REGION 1..120: note = A19-1.1 VH
QVQLVESGGGVVQPGRSLRLSCAASGFTFTNYAMHWVRQAPGKGLEWVAVISNDGSDKYYADSVKGRFTISRDNSKSTVYLQMSSLRPEDTAVYFCARDPPQVHWSLDYWGQGTLVTVSS

SEQ ID NO: 201 - protein, 8 aa, synthetic construct
  REGION 1..8: note = A19.1.1 HCDR1
GFTFTNYA

SEQ ID NO: 202 - protein, 8 aa, synthetic construct
  REGION 1..8: note = A19.1.1 HCDR2
ISNDGSDK

SEQ ID NO: 203 - protein, 15 aa, synthetic construct
  REGION 1..15: note = A19.1.1 HCDR3
CARDPPQVHWSLDYW

SEQ ID NO: 204 - protein, 110 aa, synthetic construct
  REGION 1..110: note = A19-1.1 VL
QSALTQPPSASGSPGQSVTISCTGTSSDVGDYNYVSWYQHHPGKAPKLIIYDVSKRPSGVPDRFSGSKSGDTASLTVSGLQAEDEADYYCSSYAGNNNAVFGTGTKVTVL

SEQ ID NO: 205 - protein, 9 aa, synthetic construct
  REGION 1..9: note = A19.1.1 LCDR1
SSDVGDYNY

SEQ ID NO: 206 - skipped

SEQ ID NO: 207 - protein, 12 aa, synthetic construct
  REGION 1..12: note = A19.1.1 LCDR3
CSSYAGNNNAVF

SEQ ID NO: 208 - protein, 122 aa, synthetic construct
  REGION 1..122: note = A19-30.1 VH
QVQLVESGGGVVQPGRSLRLSCAASGFTFSNYGMHWVRQAPGKGLEWLAVISYDGSNKYYADSVKGRFTISRDNSKNTLFLQMNSLRAEDTAVYYCAKESQFGELFEALDYWGQGTLVTVSS

SEQ ID NO: 209 - protein, 8 aa, synthetic construct
  REGION 1..8: note = A19-30.1 HCDR1
GFTFSNYG

SEQ ID NO: 210 - protein, 8 aa, synthetic construct
  REGION 1..8: note = A19-30.1 HCDR2
ISYDGSNK

SEQ ID NO: 211 - protein, 16 aa, synthetic construct
  REGION 1..16: note = A19-30.1 HCDR3
CAKESQFGELFEALDY

SEQ ID NO: 212 - protein, 108 aa, synthetic construct
  REGION 1..108: note = A19-30.1 VL
SYELTQPPSVSVSPGQTARITCSGDALPRKYAFWYQQKSGQAPVLVISEDSKRPSGIPERFSGSSSGTMATLTISGAQVEDEADYYCYSTDSSGNHRVFGGGTKLTVL

SEQ ID NO: 213 - protein, 6 aa, synthetic construct
  REGION 1..6: note = A19-30.1 LCDR1
ALPRKY

SEQ ID NO: 214 - skipped

SEQ ID NO: 215 - protein, 13 aa, synthetic construct [sequence omitted]

SEQ ID NO: 216 - protein, 123 aa, synthetic construct
  REGION 1..123: note = A20-36.1- VH
QVQLVESGGGVVQPGRSLRLSCAASGFIFSSYGVHWVRQAPGKGLEWVAVIWHDESNKDYADSVKGRFTISRDNSKNTVYLQMNSLRAEDTAMYYCARDGYDFLTGAYELDYWGQGTLVTVSS

SEQ ID NO: 217 - protein, 8 aa, synthetic construct
  REGION 1..8: note = A20-36.1 HCDR1
GFIFSSYG

SEQ ID NO: 218 - protein, 8 aa, synthetic construct
  REGION 1..8: note = A20-36.1 HCDR2
IWHDESNK

SEQ ID NO: 219 - protein, 18 aa, synthetic construct
  REGION 1..18: note = A20-36.1 HCDR3
CARDGYDFLTGAYELDYW

SEQ ID NO: 220 - protein, 107 aa, synthetic construct
  REGION 1..107: note = A20-36.1 VL
SYELTQPPSVSVSPGQTARITCSGDALPKQYAYWYQQKPGQAPVVVIYKDSERPSGIPERFSGSSSGTTVTLTISGVQAEDEADYYCQSADSSGTWVFGGGTKLTVL

SEQ ID NO: 221 - protein, 6 aa, synthetic construct
  REGION 1..6: note = A20-36.1 LCDR1
ALPKQY

SEQ ID NO: 222 - skipped

SEQ ID NO: 223 - protein, 12 aa, synthetic construct [sequence omitted]

SEQ ID NO: 224 - protein, 122 aa, synthetic construct
  REGION 1..122: note = A23-97.1 -VH
QVQLVESGGGVVQPGGSLRLSCAASGFTFSSFGMHWVRQAPGKGLEWVAFIRYDGSNKYYADSVKGRFTISRDNSKNTLYLQMNSLRAEDTAVYYCAKTELYYYDSSGPLGWGQGTLVTVSS

SEQ ID NO: 225 - protein, 8 aa, synthetic construct
  REGION 1..8: note = A23-97.1 HCDR1
GFTFSSFG

SEQ ID NO: 226 - protein, 8 aa, synthetic construct
  REGION 1..8: note = A23-97.1 HCDR2
IRYDGSNK

SEQ ID NO: 227 - protein, 17 aa, synthetic construct
  REGION 1..17: note = A23-97.1 HCDR3
CAKTELYYYDSSGPLGW

SEQ ID NO: 228 - protein, 107 aa, synthetic construct
  REGION 1..107: note = A23-97.1 VL
DIQMTQSPSTLSASVGDRVTITCRASQSITSWLAWYQQKPGKAPKLLIYDASSLESGVPSRFSGSGSGTEFTLTISSLQPDDFATYYCQQYNSYPWTFGQGTKVEIK

SEQ ID NO: 229 - protein, 6 aa, synthetic construct
  REGION 1..6: note = A23-97.1 LCDR1
QSITSW

SEQ ID NO: 230 - skipped

SEQ ID NO: 231 - protein, 11 aa, synthetic construct
  REGION 1..11: note = A23-97.1 LCDR3
CQQYNSYPWTF

SEQ ID NO: 232 - protein, 120 aa, synthetic construct
  REGION 1..120: note = A23-113.1 VH
QVHLEESGGAVVQPGRSLRLSCAASGFTFSSYGMHWVRQAPGKGLEWVAVISHDGSYKYYADSVKGRFTISRDNSKDTLYLQTNSLRAEDTAMYYCAKSYGYWMAYFDYWGQGTLVTVSS

SEQ ID NO: 233 - protein, 8 aa, synthetic construct
  REGION 1..8: note = A23-113.1 HCDR1
GFTFSSYG

SEQ ID NO: 234 - protein, 8 aa, synthetic construct
  REGION 1..8: note = A23-113.1 HCDR2
ISHDGSYK

SEQ ID NO: 235 - protein, 15 aa, synthetic construct
  REGION 1..15: note = A23-113.1 HCDR3
CAKSYGYWMAYFDYW

SEQ ID NO: 236 - protein, 107 aa, synthetic construct
  REGION 1..107: note = A23-113.1 VL
DIQMTQSPSSLSASVGDRVTITCRASQDISNYLAWYQQKPGKVPKLLIYAASTLQSGVPSRFSGSGSGTDFTLTISSLQPEDVATYYCQKYNSPWHTFGGGTKVEIK

SEQ ID NO: 237 - protein, 6 aa, synthetic construct
  REGION 1..6: note = A23-113.1 LCDR1
QDISNY

SEQ ID NO: 238 - skipped

SEQ ID NO: 239 - protein, 11 aa, synthetic construct
  REGION 1..11: note = A23-113.1 LCDR3
CQKYNSPWHTF

SEQ ID NO: 240 - protein, 125 aa, synthetic construct
  REGION 1..125: note = A23-80.1 VH
QVQLVQSGAEVKKPGASVKVSCKASGYTFTSNGVTWVRQAPGQGLEWMGWISTYNGDTNYAQKLQGRVSMTTDTSTRTVYMELRSLRSDDTAVYYCARVGDAYCSGGSCYHFDYWGQGTLVTVSS

SEQ ID NO: 241 - protein, 8 aa, synthetic construct
  REGION 1..8: note = A23-80.1 HCDR1
GYTFTSNG

SEQ ID NO: 242 - protein, 8 aa, synthetic construct
  REGION 1..8: note = A23-80.1 HCDR2
ISTYNGDT

SEQ ID NO: 243 - protein, 20 aa, synthetic construct
  REGION 1..20: note = A23-80.1 HCDR3
CARVGDAYCSGGSCYHFDYW

SEQ ID NO: 244 - protein, 109 aa, synthetic construct
  REGION 1..109: note = A23-80.1 VL
EIVMTQSPATLSVSPGERATLSCRASQSVSTNLAWYQQKPGQAPRLLIYGASTRATGIPARFSGSGSGTEFTLTISSLQSEDFALYYCQQYDNWPPEFTFGPGTKVDIK

SEQ ID NO: 245 - protein, 6 aa, synthetic construct
  REGION 1..6: note = A23-80.1 LCDR1
QSVSTN

SEQ ID NO: 246 - skipped

SEQ ID NO: 247 - protein, 13 aa, synthetic construct
  REGION 1..13: note = A23-80.1 LCDR3
CQQYDNWPPEFTF

SEQ ID NO: 248 - protein, 122 aa, synthetic construct
  REGION 1..122: note = A19-82.1 - VH
QVQLVESGGGVVQPGRSLRLSCVVSGLIFSTYDMHWVRQAPGKGLEWVAVISYDGSYKHYADSVKGRFTISRDSSKNTLYLQMNSLRPEDTAVYYCAKGEGVVAGTGKFDYWGQGTLVTVSS

SEQ ID NO: 249 - protein, 8 aa, synthetic construct
  REGION 1..8: note = A19-82.1 HCDR1
GLIFSTYD

SEQ ID NO: 250 - protein, 8 aa, synthetic construct
  REGION 1..8: note = A19-82.1 HCDR2
ISYDGSYK

SEQ ID NO: 251 - protein, 17 aa, synthetic construct
  REGION 1..17: note = A19-82.1 HCDR3
CAKGEGVVAGTGKFDYW

SEQ ID NO: 252 - protein, 108 aa, synthetic construct
  REGION 1..108: note = A19.82.1- VL
SYVLTQPPSVSVAPGQTARITCGGNNIGSKSVHWYQQKPGQAPVLVVYDDSDRPSGIPERFSGSNSGNTATLTISRVEAGDEADYYCQVWDGSGDPWVFGGGTKLTVL

SEQ ID NO: 253 - protein, 6 aa, synthetic construct
  REGION 1..6: note = A19-82.1 LCDR1
NIGSKS

SEQ ID NO: 254 - skipped

SEQ ID NO: 255 - protein, 13 aa, synthetic construct
  REGION 1..13: note = A19-82.1 LCDR3
CQVWDGSGDPWVF

SEQ ID NO: 256 - protein, 123 aa, synthetic construct
  REGION 1..123: note = A20-9.1 VH
QVQLVESGGGVVQPGRSLRLSCAASGFTFSSYGMHWVRQAPGKGLEWVAFISYDGSNKYYADSVKGRFTISRDNSKNTLYLQMNSLRAEDTAVYYCAKDYWSVAAGTSWFDPWGQGTLVSVSS

SEQ ID NO: 257 - protein, 8 aa, synthetic construct
  REGION 1..8: note = A20-9.1 HCDR1
GFTFSSYG

SEQ ID NO: 258 - protein, 8 aa, synthetic construct
  REGION 1..8: note = A20-9.1 HCDR2
ISYDGSNK

SEQ ID NO: 259 - protein, 18 aa, synthetic construct
  REGION 1..18: note = A20-9.1 HCDR3
CAKDYWSVAAGTSWFDPW

SEQ ID NO: 260 - protein, 107 aa, synthetic construct; identical to SEQ ID NO: 220
  REGION 1..107: note = A20-9.1 VL

SEQ ID NO: 261 - protein, 107 aa, synthetic construct; identical to SEQ ID NO: 220
  REGION 1..107: note = A20-9.1 LCDR1

SEQ ID NO: 262 - skipped

SEQ ID NO: 263 - protein, 12 aa, synthetic construct [sequence omitted]

SEQ ID NO: 264 - protein, 123 aa, synthetic construct; identical to SEQ ID NO: 1
  REGION 1..123: note = A23-58.1HC/58.1LC_93VGLTG Antibody 45 VH

SEQ ID NO: 265 - protein, 8 aa, synthetic construct
  REGION 1..8: note = A23-58.1HC/58.1LC_93VGLTG Antibody 45 HCDR1
GFTFTSSA

SEQ ID NO: 266 - protein, 8 aa, synthetic construct
  REGION 1..8: note = A23-58.1HC/58.1LC_93VGLTG Antibody 45 HCDR2
IVVGSGNT

SEQ ID NO: 267 - protein, 18 aa, synthetic construct
  REGION 1..18: note = A23-58.1HC/58.1LC_93VGLTG Antibody 45 HCDR3
CAAPNCSNVVCYDGFDIW

SEQ ID NO: 268 - protein, 109 aa, synthetic construct; identical to SEQ ID NO: 179
  REGION 1..109: note = A23-58.1HC/58.1LC_93VGLTG Antibody 45 VL

SEQ ID NO: 269 - protein, 7 aa, synthetic construct
  REGION 1..7: note = A23-58.1HC/58.1LC_93VGLTG Antibody 45 LCDR1
QSVSSSY

SEQ ID NO: 270 - skipped

SEQ ID NO: 271 - protein, 12 aa, synthetic construct
  REGION 1..12: note = A23-58.1HC/58.1LC_93VGLTG Antibody 45 LCDR3
CQQYVGLTGWTF

SEQ ID NO: 272 - protein, 123 aa, synthetic construct; identical to SEQ ID NO: 1
  REGION 1..123: note = A23-58.1HC/58.1LC_94GLTG Antibody 44 - VH

SEQ ID NO: 273 - protein, 8 aa, synthetic construct
  REGION 1..8: note = A23-58.1HC/58.1LC_94GLTG Antibody 44 HCDR1
GFTFTSSA

SEQ ID NO: 274 - protein, 8 aa, synthetic construct
  REGION 1..8: note = A23-58.1HC/58.1LC_94GLTG Antibody 44 HCDR2
IVVGSGNT

SEQ ID NO: 275 - protein, 18 aa, synthetic construct
  REGION 1..18: note = A23-58.1HC/58.1LC_94GLTG Antibody 44 HCDR3
CAAPNCSNVVCYDGFDIW

SEQ ID NO: 276 - protein, 109 aa, synthetic construct; identical to SEQ ID NO: 188
  REGION 1..109: note = A23-58.1HC/58.1LC_94GLTG Antibody 44 VL

SEQ ID NO: 277 - protein, 7 aa, synthetic construct
  REGION 1..7: note = A23-58.1HC/58.1LC_94GLTG Antibody 44 LCDR1
QSVSSSY

SEQ ID NO: 278 - skipped

SEQ ID NO: 279 - protein, 12 aa, synthetic construct
  REGION 1..12: note = A23-58.1HC/58.1LC_94GLTG Antibody 44 LCDR3
CQQYGGLTGWTF

SEQ ID NO: 280 - protein, 123 aa, synthetic construct; identical to SEQ ID NO: 1
  REGION 1..123: note = A23-58.1HC/S2E12LC Antibody 56 - VH

SEQ ID NO: 281 - protein, 8 aa, synthetic construct
  REGION 1..8: note = A23-58.1HC/S2E12LC Antibody 56 HCDR1
GFTFTSSA

SEQ ID NO: 282 - protein, 8 aa, synthetic construct
  REGION 1..8: note = A23-58.1HC/S2E12LC Antibody 56 HCDR2
IVVGSGNT

SEQ ID NO: 283 - protein, 18 aa, synthetic construct
  REGION 1..18: note = A23-58.1HC/S2E12LC Antibody 56 HCDR3
CAAPNCSNVVCYDGFDIW

SEQ ID NO: 284 - protein, 109 aa, synthetic construct; identical to SEQ ID NO: 189
  REGION 1..109: note = A23-58.1HC/S2E12LC Antibody 56 VL

SEQ ID NO: 285 - protein, 7 aa, synthetic construct
  REGION 1..7: note = A23-58.1HC/S2E12LC Antibody 56 LCDR1
QSVSSSY

SEQ ID NO: 286 - skipped

SEQ ID NO: 287 - protein, 12 aa, synthetic construct
  REGION 1..12: note = A23-58.1HC/S2E12LC Antibody 56 LCDR3
CQQYVGLTGWTF

SEQ ID NO: 288 - protein, 123 aa, synthetic construct; identical to SEQ ID NO: 190
  REGION 1..123: note = A23-58.1HC_Y100cA/S2E12LC Antibody 57 - VH

SEQ ID NO: 289 - protein, 8 aa, synthetic construct
  REGION 1..8: note = A23-58.1HC_Y100cA/S2E12LC Antibody 57 HCDR1
GFTFTSSA

SEQ ID NO: 290 - protein, 8 aa, synthetic construct
  REGION 1..8: note = A23-58.1HC_Y100cA/S2E12LC Antibody 57 HCDR2
IVVGSGNT

SEQ ID NO: 291 - protein, 18 aa, synthetic construct
  REGION 1..18: note = A23-58.1HC_Y100cA/S2E12LC Antibody 57 HCDR3
CAAPNCSNVVCADGFDIW

SEQ ID NO: 292 - protein, 109 aa, synthetic construct; identical to SEQ ID NO: 189
  REGION 1..109: note = A23-58.1HC_Y100cA/S2E12LC Antibody 57 VL

SEQ ID NO: 293 - protein, 7 aa, synthetic construct
  REGION 1..7: note = A23-58.1HC_Y100cA/S2E12LC Antibody 57 LCDR1
QSVSSSY

SEQ ID NO: 294 - skipped

SEQ ID NO: 295 - protein, 12 aa, synthetic construct
  REGION 1..12: note = A23-58.1HC_Y100cA/S2E12LC Antibody 57 LCDR3
CQQYVGLTGWTF

SEQ ID NO: 296 - protein, 123 aa, synthetic construct; identical to SEQ ID NO: 17
  REGION 1..123: note = B1-182.1HC/182.1LC_94GLTG Antibody 27 - VH

SEQ ID NO: 297 - protein, 8 aa, synthetic construct
  REGION 1..8: note = B1-182.1HC/182.1LC_94GLTG Antibody 27 HCDR1
GFTFTSSA

SEQ ID NO: 298 - protein, 8 aa, synthetic construct
  REGION 1..8: note = B1-182.1HC/182.1LC_94GLTG Antibody 27 HCDR2
IVVGSGNT

SEQ ID NO: 299 - protein, 18 aa, synthetic construct
  REGION 1..18: note = B1-182.1HC/182.1LC_94GLTG Antibody 27 HCDR3
CAAPYCSGGSCFDGFDIW

SEQ ID NO: 300 - protein, 109 aa, synthetic construct; identical to SEQ ID NO: 191
  REGION 1..109: note = B1-182.1HC/182.1LC_94GLTG Antibody 27 VL

SEQ ID NO: 301 - protein, 7 aa, synthetic construct
  REGION 1..7: note = B1-182.1HC/182.1LC_94GLTG Antibody 27 LCDR1
QSVSSSY

SEQ ID NO: 302 - skipped

SEQ ID NO: 303 - protein, 12 aa, synthetic construct
  REGION 1..12: note = B1-182.1HC/182.1LC_94GLTG Antibody 27 LCDR3
CQQYGGLTGWTF

SEQ ID NO: 304 - protein, 118 aa, synthetic construct
  REGION 1..118: note = VH SARS2.F768_pt2_G10 (G10)
EVQLVETGGGLIQPGGSLRLSCAASGVIVSRNYMSWVRQAPGKGLEWVSLMFAGGSAFYSDSVKGRFTISRDDSRNTLYLQMNSLRAEDTAVYYCARDLDVRGGLDVWGQGTTVTVSS

SEQ ID NO: 305 - protein, 105 aa, synthetic construct
  REGION 1..105: note = VL SARS2.F768_pt2_G10 (G10)
DVQLTQFPSSLSASVGDRVTITCRASQDIPNYINWYQQKPGKAPRLLIYAASTLQSGVPSRFSGSGSGTDFTLTISSLQPEDFGTYYCQQSDTRTFGPGTKLEIK

SEQ ID NO: 306 - protein, 118 aa, synthetic construct
  REGION 1..118: note = VH - SARS2.F768_pt1_D11
EVQLVESGGGLVQPGGSLRLSCAASEIIVTRNYMTWVRQAPGKGLEWVSLIYPGGTTYYADSVKGRFTISRDNSKNTLYLQMNSLTTEDTAVYFCARDLDIRGGVDIWGQGTLVTVSS

SEQ ID NO: 307 - protein, 107 aa, synthetic construct
  REGION 1..107: note = VL - SARS2.F768_pt1_D11
DIQMTQSPSSLSASVGDRVSITCRASQDINNYLNWYQQKPGKAPRLLIFAASSLQSGVPSRFSGSGSGTDFTLTITSLQPEDFATYYCHQSSTTPWTFGQGTKVEIK

SEQ ID NO: 308 - protein, 118 aa, synthetic construct
  REGION 1..118: note = VH - SARS2.F768_pt1_A05
EVQLVETGGGLIQPGGSLRLSCAASGVIVSRNYMSWVRQAPGKGLEWVSLLFAGGSAFYSDSVKGRFTISRDDSRNTLYLQMNSLRAEDTAVYYCARDLDVRGGLDVWGQGTTVTVSS

SEQ ID NO: 309 - protein, 105 aa, synthetic construct
  REGION 1..105: note = VL - SARS2.F768_pt1_A05
DVQLTQFPSSLSASVGDRVTITCRAGQGIPNYISWYHQRPGKAPRLLIYAASTLQSGVPSRFSGSGSGTDFTLTISSLQPEDFGTYYCQQSDTRTFGPGTKLEIK

SEQ ID NO: 310 - protein, 117 aa, synthetic construct
  REGION 1..117: note = VH - SARS2.F768_pt2_D12
EVQLVESGGGLVQPGGSLRLSCAASEIIVNRNYMSWVRQAPGQGLEWVSLLYAGGSTFYADSVKGRFAISRDDSKNTLYLQMNNLRGDDTAVYYCARDLIDYGMDVWGQGTTVTVSS

SEQ ID NO: 311 - protein, 108 aa, synthetic construct
  REGION 1..108: note = VL - SARS2.F768_pt2_D12
DIQLTQSPSFLSASVGDRVTMTCRASQGISTYLAWYQQKPGKAPKLLIYAASTLQSGVPSRFSGSGSGTEFTLTISSLQPEDFATYYCQQVNSSPPVTFGQGTRLEIK

SEQ ID NO: 312 - protein, 132 aa, synthetic construct
  REGION 1..132: note = VH - SARS2.F770_pt1_G11
EVQLVESGGGRVKPGGSLRLSCAASGFTFSSYRMNWVRQAPGKGLEWVSSISSGSSYIYFADSVKGRFTISRDNAKNSLFLRMNNLRVEDTAVYYCARAPTLRHSSGWYDPPGTENSSFDLWGRGTLVTVSS

SEQ ID NO: 313 - protein, 108 aa, synthetic construct
  REGION 1..108: note = VL - SARS2.F770_pt1_G11
SYVLTQPPSVSVAPGQTARITCGGNNIGTKSVHWYQQKPGQAPVLVISSDSDRPSGVPDRFSGSNSGNTATLTISRVEAGDEADYYCHVWDSSSDHWVFGGGTKLTVL

SEQ ID NO: 314 - protein, 120 aa, synthetic construct
  REGION 1..120: note = VH - SARS2.F769_pt1_B1
QVQLVQSGAEVKEPGASVKVSCKTSGYTFTTYLIHWVRQAPGQGLEWMGVTNPNGDGSSYALNFQGRVTMTSDTSTSTVYMELSSLRSEDTAIYYCARVDDFWSGYWDYWGQGTLVTVST

SEQ ID NO: 315 - protein, 113 aa, synthetic construct
  REGION 1..113: note = VL - SARS2.F769_pt1_B1
QSVLTQPPSVSGAPGQRVTISCTGISSNIGAGYDVHWYQQHPGTAPRLLIYDNTNRPSGVPDRFSGSKSGTSASLAITGLQAEDEADYFCQAYVTGLISSGYVFGTGTKVSVL

SEQ ID NO: 316 - protein, 119 aa, synthetic construct
  REGION 1..119: note = VH - SARS2.F770_pt1_C1
QITLKESGPTLVKRKQTLTLTCTFSGFAFSASGVGVGWVRQPPGKGLEWLALIYWDDDKKYSPSLKSRLSITKDTSKNQVVLTLTNVDALDTGTYYCAHHTVPTIFDFWGQGTLVAVSS

SEQ ID NO: 317 - protein, 111 aa, synthetic construct
  REGION 1..111: note = VL - SARS2.F770_pt1_C1
QSALTQPASVSGSPGQSITISCTGTSSDVGDHNYVSWYQRHPGKAPKLMIYEVTNRPSGVSNRFSGSKSGNTASLTISGLQAEDEADYFCSSYSTSSTPVVFGGGTKLTVL

SEQ ID NO: 318 - protein, 124 aa, synthetic construct
  REGION 1..124: note = VH - SARS2-A20-29.1
EVQLVESGGGLVQPGRSLRLSCAASGFTFDDYAMHWVRQTPGKGLEWVSGISWNSGDIDYADSVKGRFTISRDNAKNSLYLQMNSLRTEDTALYYCTKGWFGEFFGAGSICDYWGQGTLVTVSS

SEQ ID NO: 319 - protein, 8 aa, synthetic construct
  REGION 1..8: note = HCDR1 - SARS2-A20-29.1
GFTFDDYA

SEQ ID NO: 320 - protein, 8 aa, synthetic construct
  REGION 1..8: note = HCDR2 - SARS2-A20-29.1
ISWNSGDI

SEQ ID NO: 321 - protein, 19 aa, synthetic construct
  REGION 1..19: note = HCDR3 - SARS2-A20-29.1
CTKGWFGEFFGAGSICDYW

SEQ ID NO: 322 - protein, 106 aa, synthetic construct
  REGION 1..106: note = VL - SARS2-A20-29.1
EIVMTQSPATLSVSPGERATLSCRASQSVSNNLAWYQQKPGQAPRLLIYGASTRATGIPARFSGSGSGTEFTLTISSLQSEDFAVYYCQQYNNWPLFGQGTKVEIK

SEQ ID NO: 323 - protein, 6 aa, synthetic construct
  REGION 1..6: note = LCDR1 - SARS2-A20-29.1
QSVSNN

SEQ ID NO: 324 - skipped

SEQ ID NO: 325 - protein, 10 aa, synthetic construct [sequence omitted]

SEQ ID NO: 326 - protein, 127 aa, synthetic construct; identical to SEQ ID NO: 25
  REGION 1..127: note = VH - SARS2-A19-61.1

SEQ ID NO: 327 - protein, 8 aa, synthetic construct
  REGION 1..8: note = HCDR1 - SARS2-A19-61.1
GFTFSSYA

SEQ ID NO: 328 - protein, 8 aa, synthetic construct
  REGION 1..8: note = HCDR2 - SARS2-A19-61.1
ISYDGSNQ

SEQ ID NO: 329 - protein, 22 aa, synthetic construct
  REGION 1..22: note = HCDR3 - SARS2-A19-61.1
CARDLAIAVAGTWHYYNGMDVW

SEQ ID NO: 330 - protein, 107 aa, synthetic construct; identical to SEQ ID NO: 29
  REGION 1..107: note = VL - SARS2-A19-61.1

SEQ ID NO: 331 - protein, 6 aa, synthetic construct
  REGION 1..6: note = LCDR1 - SARS2-A19-61.1
QGISSW

SEQ ID NO: 332 - skipped

SEQ ID NO: 333 - protein, 11 aa, synthetic construct [sequence omitted]

SEQ ID NO: 334 - protein, 123 aa, synthetic construct; identical to SEQ ID NO: 1
  REGION 1..123: note = VH - SARS2-A23-58.1

SEQ ID NO: 335 - protein, 8 aa, synthetic construct
  REGION 1..8: note = HCDR1 - SARS2-A23-58.1
GFTFTSSA

SEQ ID NO: 336 - protein, 8 aa, synthetic construct
  REGION 1..8: note = HCDR2 - SARS2-A23-58.1
IVVGSGNT

SEQ ID NO: 337 - protein, 18 aa, synthetic construct
  REGION 1..18: note = HCDR3 - SARS2-A23-58.1
CAAPNCSNVVCYDGFDIW

SEQ ID NO: 338 - protein, 108 aa, synthetic construct; identical to SEQ ID NO: 5
  REGION 1..108: note = VL - SARS2-A23-58.1

SEQ ID NO: 339 - protein, 7 aa, synthetic construct
  REGION 1..7: note = LCDR1 - SARS2-A23-58.1
QSVSSSY

SEQ ID NO: 340 - skipped

SEQ ID NO: 341 - protein, 11 aa, synthetic construct [sequence omitted]

SEQ ID NO: 342 - protein, 123 aa, synthetic construct; identical to SEQ ID NO: 17
  REGION 1..123: note = VH - SARS2-B1-182.1

SEQ ID NO: 343 - protein, 8 aa, synthetic construct
  REGION 1..8: note = HCDR1 - SARS2-B1-182.1
GFTFTSSA

SEQ ID NO: 344 - protein, 8 aa, synthetic construct
  REGION 1..8: note = HCDR2 - SARS2-B1-182.1
IVVGSGNT

SEQ ID NO: 345 - protein, 18 aa, synthetic construct
  REGION 1..18: note = HCDR3 - SARS2-B1-182.1
CAAPYCSGGSCFDGFDIW

SEQ ID NO: 346 - protein, 108 aa, synthetic construct; identical to SEQ ID NO: 21
  REGION 1..108: note = VL - SARS2-B1-182.1

SEQ ID NO: 347 - protein, 7 aa, synthetic construct
  REGION 1..7: note = LCDR1 - SARS2-B1-182.1
QSVSSSY

SEQ ID NO: 348 - skipped

SEQ ID NO: 349 - protein, 11 aa, synthetic construct [sequence omitted]

SEQ ID NO: 350 - DNA, 375 bp, synthetic construct
  misc_feature 1..375: note = E8 VH wild type (N62) DNA
  source 1..375: mol_type = other DNA
caggtcacactgcgcgagtctgggcctgctctggttaagccaacgcaaaccctgacactcacctgcactttttcgggtttcagtttgtcaacacgtggcgtttgcgtttcctggatccgccaaccacctggcaaagccctggagtggctggctttcatcgactgggacgatgacaagtactataacactagcttgaaaactcggctcacaatttcaaaggacacctccaagaaccaggtggtgctgaccatgaccaatgtccatcctgtggatacagccacttattattgtgcaagaatttctagccaccacgggtcttcctggtgcacgacctttgattactggggccagggagcactcgttaccgtctctgcc

SEQ ID NO: 351 - protein, 125 aa, synthetic construct
  REGION 1..125: note = E8 VH N62Q peptide
QVTLRESGPALVKPTQTLTLTCTFSGFSLSTRGVCVSWIRQPPGKALEWLAFIDWDDDKYYQTSLKTRLTISKDTSKNQVVLTMTNVHPVDTATYYCARISSHHGSSWCTTFDYWGQGALVTVSA

SEQ ID NO: 352 - DNA, 375 bp, synthetic construct
  misc_feature 1..375: note = E8 VH N62Q DNA
  source 1..375: mol_type = other DNA
caggtcacactgcgcgagtctgggcctgctctggttaagccaacgcaaaccctgacactcacctgcactttttcgggtttcagtttgtcaacacgtggcgtttgcgtttcctggatccgccaaccacctggcaaagccctggagtggctggctttcatcgactgggacgatgacaagtactatcagactagcttgaaaactcggctcacaatttcaaaggacacctccaagaaccaggtggtgctgaccatgaccaatgtccatcctgtggatacagccacttattattgtgcaagaatttctagccaccacgggtcttcctggtgcacgacctttgattactggggccagggagcactcgttaccgtctctgcc

SEQ ID NO: 353 - protein, 125 aa, synthetic construct
  REGION 1..125: note = E8 VH N62S peptide
QVTLRESGPALVKPTQTLTLTCTFSGFSLSTRGVCVSWIRQPPGKALEWLAFIDWDDDKYYSTSLKTRLTISKDTSKNQVVLTMTNVHPVDTATYYCARISSHHGSSWCTTFDYWGQGALVTVSA

SEQ ID NO: 354 - DNA, 375 bp, synthetic construct
  misc_feature 1..375: note = E8 VH N62S DNA
  source 1..375: mol_type = other DNA
caggtcacactgcgcgagtctgggcctgctctggttaagccaacgcaaaccctgacactcacctgcactttttcgggtttcagtttgtcaacacgtggcgtttgcgtttcctggatccgccaaccacctggcaaagccctggagtggctggctttcatcgactgggacgatgacaagtactatagcactagcttgaaaactcggctcacaatttcaaaggacacctccaagaaccaggtggtgctgaccatgaccaatgtccatcctgtggatacagccacttattattgtgcaagaatttctagccaccacgggtcttcctggtgcacgacctttgattactggggccagggagcactcgttaccgtctctgcc

SEQ ID NO: 355 - protein, 220 aa, synthetic construct
  REGION 1..220: note = E8 VL in expression plasmid - peptide
DTVMTQSPLSLPVTPGEPASISCRSSQSLLNSNGYNYLDWYVQKPGQSPQLLIYLGSNRASGVPDRFSGSGSGTDFTLKISRVEAEDVGVYYCMQPLQTPLFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - DNA, 660 bp, synthetic construct
  misc_feature 1..660: note = E8 VL in expression plasmid - DNA
  source 1..660: mol_type = other DNA
gataccgtcatgacccagagccctctgagtctacccgtcactcctggagaaccagcctccatctcctgtcgaagtagtcaaagtctcctgaactccaatggctacaactatctggactggtacgtccaaaaacccggacaaagcccacaactcctgatctatctgggtagcaaccgggcaagcggcgtgcccgataggttcagtggctcagggtcgggaaccgacttcaccctcaaaatcagcagggttgaggctgaagatgttggagtgtactactgcatgcaaccgctccaaactccattgttcacatttgggcccggaaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 357 - protein, 455 aa, synthetic construct
  REGION 1..455: note = E8 VH N62Q in expression plasmid peptide
QVTLRESGPALVKPTQTLTLTCTFSGFSLSTRGVCVSWIRQPPGKALEWLAFIDWDDDKYYNTSLKTRLTISKDTSKNQVVLTMTNVHPVDTATYYCARISSHHGSSWCTTFDYWGQGALVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 358 - DNA, 1365 bp, synthetic construct
  misc_feature 1..1365: note = E8 VH N62Q in expression plasmid DNA
  source 1..1365: mol_type = other DNA
caggtcacactgcgcgagtctgggcctgctctggttaagccaacgcaaaccctgacactcacctgcactttttcgggtttcagtttgtcaacacgtggcgtttgcgtttcctggatccgccaaccacctggcaaagccctggagtggctggctttcatcgactgggacgatgacaagtactataacactagcttgaaaactcggctcacaatttcaaaggacacctccaagaaccaggtggtgctgaccatgaccaatgtccatcctgtggatacagccacttattattgtgcaagaatttctagccaccacgggtcttcctggtgcacgacctttgattactggggccagggagcactcgttaccgtctctgccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcacagccactacacgcagaagagcctctccctgtctccgggcaaa

SEQ ID NO: 359 - protein, 455 aa, synthetic construct
  REGION 1..455: note = E8 VH N62S in expression plasmid peptide
QVTLRESGPALVKPTQTLTLTCTFSGFSLSTRGVCVSWIRQPPGKALEWLAFIDWDDDKYYSTSLKTRLTISKDTSKNQVVLTMTNVHPVDTATYYCARISSHHGSSWCTTFDYWGQGALVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 360 - DNA, 1365 bp, synthetic construct
  misc_feature 1..1365: note = E8 VH N62S in expression plasmid DNA
  source 1..1365: mol_type = other DNA
caggtcacactgcgcgagtctgggcctgctctggttaagccaacgcaaaccctgacactcacctgcactttttcgggtttcagtttgtcaacacgtggcgtttgcgtttcctggatccgccaaccacctggcaaagccctggagtggctggctttcatcgactgggacgatgacaagtactattccactagcttgaaaactcggctcacaatttcaaaggacacctccaagaaccaggtggtgctgaccatgaccaatgtccatcctgtggatacagccacttattattgtgcaagaatttctagccaccacgggtcttcctggtgcacgacctttgattactggggccagggagcactcgttaccgtctctgccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctccatgaggctctgcacagccactacacgcagaagagcctctccctgtctccgggcaaa

(19) United States
(12) Patent Application Publication
(10) Pub. No.: US 2026/0327292 A1
(43) Pub. Date: Oct. 22, 2026

(54) ANTI-SARS-COV-2 ANTIGEN BINDING POLYPEPTIDES, POLYPEPTIDE COMPLEXES AND METHODS OF USE THEREOF

(71) Applicants: ModeX Therapeutics, Inc., Weston, MA (US); The United States of America, as Represented by the Secretary, Dept. of Health and Human Services, Bethesda, MD (US)

(72) Inventors: Ronnie R. WEI, Weston, MA (US); Chi-Jen WEI, Weston, MA (US); Zhi-Yong YANG, Newton Center, MA (US); John R. MASCOLA, Rockville, MD (US); Gary J. NABEL, Delray Beach, FL (US); John MISASI, Bethesda, MD (US); Amarendra PEGU, Bethesda, MD (US); Lingshu WANG, Bethesda, MD (US); Tongqing ZHOU, Bethesda, MD (US); Misook CHOE, Bethesda, MD (US); Olamide K. OLONINIYI, Bethesda, MD (US); Bingchun ZHAO, Bethesda, MD (US); Yi ZHANG, Bethesda, MD (US); Eun Sung YANG, Bethesda, MD (US); Man CHEN, Bethesda, MD (US); Kwanyee LEUNG, Bethesda, MD (US); Wei SHI, Bethesda, MD (US); Nancy J. SULLIVAN, Bethesda, MD (US); Peter D. KWONG, Bethesda, MD (US); Richard A. KOUP, Bethesda, MD (US); Barney S. GRAHAM, Smyrna, GA (US)

(21) Appl. No.: 18/879,056

(22) PCT Filed: Jun. 30, 2023
(86) PCT No.: PCT/US2023/069541

§ 371 Date: Dec. 26, 2024

Related U.S. Application Data
(60) Provisional application No. 63/357,336, filed on Jun. 30, 2022.
(60) Provisional application No. 63/357,873, filed on Jul. 1, 2022.
(60) Provisional application No. 63/404,473, filed on Sep. 7, 2022.
(60) Provisional application No. 63/381,850, filed on Nov. 1, 2022.
(60) Provisional application No. 63/433,719, filed on Dec. 19, 2022.

(51) Int. Cl. C07K 16/104; A61P 31/14; G01N 33/569

(52) U.S. Cl. CPC C07K 16/104; A61P 31/14; G01N 33/56983; C07K 2317/515; C07K 2317/522; C07K 2317/524; C07K 2317/526; C07K 2317/71; C07K 2317/76; C07K 2317/92; G01N 2333/165

(57) ABSTRACT

Disclosed are antigen binding antigen binding polypeptide complexes (e.g., antibodies and antigen binding fragments thereof) having certain structural and/or functional features. Also disclosed are polynucleotides and vectors encoding such polypeptide complexes; host cells, pharmaceutical compositions and kits containing such polypeptide complexes; and methods of using such polypeptide complexes.

### CROSS REFERENCE TO RELATED APPLICATIONS

This application claims the priority benefit of U.S. Provisional Application No. 63/357,336, filed Jun. 30, 2022; U.S. Provisional Application No. 63/357,873, filed Jul. 1, 2022; U.S. Provisional Application No. 63/404,473, filed Sep. 7, 2022; U.S. Provisional Application No. 63/381,850, filed Nov. 1, 2022; and U.S. Provisional Application No. 63/433,719, filed Dec. 9, 2022; which are all incorporated herein by reference in their entireties.

### STATEMENT REGARDING FEDERALLY SPONSORED RESEARCH OR DEVELOPMENT

This invention was made in the performance of a Cooperative Research and Development Agreement with the National Institutes of Health, an Agency of the Department of Health and Human Services. The Government has certain rights in this invention.

### REFERENCE TO SEQUENCE LISTING SUBMITTED ELECTRONICALLY

The content of the electronically submitted sequence listing (Name: 4850_0100003_SEQLISTING_ST26.xml; Size: 486,871 bytes; Date of Creation: Jun. 28, 2023) is herein incorporated by reference in its entirety.

### FIELD

The present disclosure relates to anti-severe acute respiratory syndrome coronavirus 2 (anti-SARS-CoV-2) antigen binding polypeptides and polypeptide complexes (e.g., antibodies and antigen binding fragments thereof) having certain structural and/or functional features. The present disclosure also relates to polynucleotides and vectors encoding such polypeptides and polypeptide complexes; host cells; pharmaceutical compositions and kits containing such polypeptides and polypeptide complexes; and methods of using such polypeptides and polypeptide complexes.

### BACKGROUND

The outbreak of severe acute respiratory syndrome coronavirus (SARS-CoV-2) has caused over 450 million infections and over 6 million deaths worldwide. SARS-CoV-2 is the strain of coronavirus that causes COVID-19 (coronavirus disease 2019). Sequencing and publication of the original Wuhan Hu-1 (WA-1) genome enabled rapid vaccine design and production of spike proteins for therapeutic antibody development. However, persistent circulation of viruses during human pandemics leads to genetic variation that resists containment and eradication. While cellular and humoral immune responses are needed for optimal protection against viral infection, neutralizing antibodies play a major role in prevention and eradication of pathogens.

SARS-CoV-2 spike protein mutants and SARS-CoV-2 variants Alpha (B.1.1.7), Beta (B.1.351), Gamma (P.1), Delta (B.1.617.2), Epsilon variant (B.1.427), and Omicron (B.1.1.529) have emerged to contain mutations that are resistant to current therapies, have increased pathogenicity, and evade current vaccines. This led to their classification as variants of concern (VOCs).

Therapeutic antibodies have emerged as an important class of agents for treating human diseases and disorders. Therapeutic antibodies have been engineered to have specificity for two or more different antigens or epitopes (i.e., “multispecific” antibodies that are, for example, bispecific, trispecific or tetraspecific). Multispecific antibodies have been used to form multi-targeting strategies to treat human diseases and disorders, and are an important technological platform for developing neutralizing antibody-based therapeutics for preventing and treating SARS-CoV-2 infection and COVID-19.

There is a need for improved therapies to prevent and treat SARS-CoV-2 infection and COVID-19. There is a need for therapeutic antibodies with broad VOC reactivity and resistance to SARS-CoV-2 escape. There is also a need for therapeutic antibodies that are easier to manufacture and administer than current therapeutic antibody cocktails. There is also a need to treat or prevent SARS-CoV-2 infection and COVID-19 in immunocompromised individuals who cannot mount an effective immune response through vaccinations.

### BRIEF SUMMARY

Provided herein is an antigen binding polypeptide complex comprising a first polypeptide, a second polypeptide, a third polypeptide, and a fourth polypeptide; wherein the first polypeptide has a structure represented by VL1-L1-CL; wherein the second polypeptide has a structure represented by VH1-L2-CH1; wherein the third polypeptide has a structure represented by VH2-L3-VH3-L4-CH1; wherein the fourth polypeptide has a structure represented by VL3-L5-VL2-L6-CL; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; CH1 is an immunoglobulin heavy chain constant region 1; CL is an immunoglobulin light chain constant region; and L1-L6 are amino acid linkers.

Also provided herein is an antigen binding polypeptide complex comprising a first polypeptide, a second polypeptide, a third polypeptide, and a fourth polypeptide; wherein the first polypeptide has a structure represented by VL1-L1-CL; wherein the second polypeptide has a structure represented by VH1-L2-CH1-L3-Fc; wherein the third polypeptide has a structure represented by VH2-L4-VH3-L5-CH1-L6-Fc; wherein the fourth polypeptide has a structure represented by VL3-L7-VL2-L8-CL; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers.

In some aspects, VH1 comprises the same heavy chain variable region as VH3, and VL1 comprises the same light chain variable region as VL3.

In some aspects, VH1 comprises the same heavy chain variable region as VH2, and VL1 comprises the same light chain variable region as VL2.

In some aspects, VH2 comprises the same heavy chain variable region as VH3, and VL2 comprises the same light chain variable region as VL3.

In some aspects, VH1, VH2 and VH3 each comprise the same heavy chain variable region, and VL1, VL2 and VL3 each comprise the same light chain variable region.

In some aspects, VH1, VH2 and VH3 each comprise different heavy chain variable regions, and VL1, VL2 and VL3 each comprise different light chain variable regions.

In some aspects, VH1, VH2 and VH3 each independently comprise a CDR1 comprising an amino acid sequence having at least 80%, at least 85%, at least 90%, at least 95%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; a CDR2 comprising an amino acid sequence having at least 80%, at least 85%, at least 90%, at least 95%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; and a CDR3 comprising an amino acid sequence having at least 80%, at least 85%, at least 90%, at least 95%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345; and VL1, VL2 and VL3 each independently comprise a CDR1 comprising an amino acid sequence having at least 80%, at least 85%, at least 90%, at least 95%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; a CDR2 comprising an amino acid sequence having at least 80%, at least 85%, at least 90%, at least 95%, at least 99% or 100% identity to any one of SEQ ID NOs:7 (having the sequence of SAS), 15 (having the sequence of GTN), 23 (having the sequence of GAS), 31 (having the sequence of DAS), 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and a CDR3 comprising an amino acid sequence having at least 80%, at least 85%, at least 90%, at least 95%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349.

In some aspects, VH1, VH2 and VH3 each comprise a CDR1 comprising an amino acid sequence having at least 80%, at least 85%, at least 90%, at least 95%, at least 99% or 100% identity to SEQ ID NO:18; a CDR2 comprising an amino acid sequence having at least 80%, at least 85%, at least 90%, at least 95%, at least 99% or 100% identity to SEQ ID NO: 19; and a CDR3 comprising an amino acid sequence having at least 80%, at least 85%, at least 90%, at least 95%, at least 99% or 100% identity to SEQ ID NO:20; and VL1, VL2 and VL3 each comprise a CDR1 comprising an amino acid sequence having at least 80%, at least 85%, at least 90%, at least 95%, at least 99% or 100% identity to SEQ ID NO:22; a CDR2 comprising an amino acid sequence having at least 80%, at least 85%, at least 90%, at least 95%, at least 99% or 100% identity to SEQ ID NO:23 (having the sequence of GAS); and a CDR3 comprising an amino acid sequence having at least 80%, at least 85%, at least 90%, at least 95%, at least 99% or 100% identity to SEQ ID NO:24.

In some aspects, VH1 and VH3 each comprise a CDR1 comprising an amino acid sequence having at least 80%, at least 85%, at least 90%, at least 95%, at least 99% or 100% identity to SEQ ID NO:18; a CDR2 comprising an amino acid sequence having at least 80%, at least 85%, at least 90%, at least 95%, at least 99% or 100% identity to SEQ ID NO:19; and a CDR3 comprising an amino acid sequence having at least 80%, at least 85%, at least 90%, at least 95%, at least 99% or 100% identity to SEQ ID NO:20; VH2 comprises a CDR1 comprising an amino acid sequence having at least 80%, at least 85%, at least 90%, at least 95%, at least 99% or 100% identity to SEQ ID NO:26; a CDR2 comprising an amino acid sequence having at least 80%, at least 85%, at least 90%, at least 95%, at least 99% or 100% identity to SEQ ID NO:27; and a CDR3 comprising an amino acid sequence having at least 80%, at least 85%, at least 90%, at least 95%, at least 99% or 100% identity to SEQ ID NO:28; VL1 and VL3 each comprise a CDR1 comprising an amino acid sequence having at least 80%, at least 85%, at least 90%, at least 95%, at least 99% or 100% identity to SEQ ID NO:22; a CDR2 comprising an amino acid sequence having at least 80%, at least 85%, at least 90%, at least 95%, at least 99% or 100% identity to SEQ ID NO:23 (having the sequence of GAS); and a CDR3 comprising an amino acid sequence having at least 80%, at least 85%, at least 90%, at least 95%, at least 99% or 100% identity to SEQ ID NO:24; and VL2 comprises a CDR1 comprising an amino acid sequence having at least 80%, at least 85%, at least 90%, at least 95%, at least 99% or 100% identity to SEQ ID NO:30; a CDR2 comprising an amino acid sequence having at least 80%, at least 85%, at least 90%, at least 95%, at least 99% or 100% identity to SEQ ID NO:31 (having the sequence of DAS); and a CDR3 comprising an amino acid sequence having at least 80%, at least 85%, at least 90%, at least 95%, at least 99% or 100% identity to SEQ ID NO:32.

In some aspects, VH1 and VH2 each comprise a CDR1 comprising an amino acid sequence having at least 80%, at least 85%, at least 90%, at least 95%, at least 99% or 100% identity to SEQ ID NO:18; a CDR2 comprising an amino acid sequence having at least 80%, at least 85%, at least 90%, at least 95%, at least 99% or 100% identity to SEQ ID NO:19; and a CDR3 comprising an amino acid sequence having at least 80%, at least 85%, at least 90%, at least 95%, at least 99% or 100% identity to SEQ ID NO:20; VH3 comprises a CDR1 comprising an amino acid sequence having at least 80%, at least 85%, at least 90%, at least 95%, at least 99% or 100% identity to SEQ ID NO:26; a CDR2 comprising an amino acid sequence having at least 80%, at least 85%, at least 90%, at least 95%, at least 99% or 100% identity to SEQ ID NO:27; and a CDR3 comprising an amino acid sequence having at least 80%, at least 85%, at least 90%, at least 95%, at least 99% or 100% identity to SEQ ID NO:28; VL1 and VL2 each comprise a CDR1 comprising an amino acid sequence having at least 80%, at least 85%, at least 90%, at least 95%, at least 99% or 100% identity to SEQ ID NO:22; a CDR2 comprising an amino acid sequence having at least 80%, at least 85%, at least 90%, at least 95%, at least 99% or 100% identity to SEQ ID NO:23 (having the sequence of GAS); and a CDR3 comprising an amino acid sequence having at least 80%, at least 85%, at least 90%, at least 95%, at least 99% or 100% identity to SEQ ID NO:24; and VL3 comprises a CDR1 comprising an amino acid sequence having at least 80%, at least 85%, at least 90%, at least 95%, at least 99% or 100% identity to SEQ ID NO:30; a CDR2 comprising an amino acid sequence having at least 80%, at least 85%, at least 90%, at least 95%, at least 99% or 100% identity to SEQ ID NO:31 (having the sequence of DAS); and a CDR3 comprising an amino acid sequence having at least 80%, at least 85%, at least 90%, at least 95%, at least 99% or 100% identity to SEQ ID NO:32.

In some aspects, VH2 and VH3 each comprise a CDR1 comprising an amino acid sequence having at least 80%, at least 85%, at least 90%, at least 95%, at least 99% or 100% identity to SEQ ID NO:18; a CDR2 comprising an amino acid sequence having at least 80%, at least 85%, at least 90%, at least 95%, at least 99% or 100% identity to SEQ ID NO:19; and a CDR3 comprising an amino acid sequence having at least 80%, at least 85%, at least 90%, at least 95%, at least 99% or 100% identity to SEQ ID NO:20; VH1 comprises a CDR1 comprising an amino acid sequence having at least 80%, at least 85%, at least 90%, at least 95%, at least 99% or 100% identity to SEQ ID NO:26; a CDR2 comprising an amino acid sequence having at least 80%, at least 85%, at least 90%, at least 95%, at least 99% or 100% identity to SEQ ID NO:27; and a CDR3 comprising an amino acid sequence having at least 80%, at least 85%, at least 90%, at least 95%, at least 99% or 100% identity to SEQ ID NO:28; VL2 and VL3 each comprise a CDR1 comprising an amino acid sequence having at least 80%, at least 85%, at least 90%, at least 95%, at least 99% or 100% identity to SEQ ID NO:22; a CDR2 comprising an amino acid sequence having at least 80%, at least 85%, at least 90%, at least 95%, at least 99% or 100% identity to SEQ ID NO:23 (having the sequence of GAS); and a CDR3 comprising an amino acid sequence having at least 80%, at least 85%, at least 90%, at least 95%, at least 99% or 100% identity to SEQ ID NO:24; and VL1 comprises a CDR1 comprising an amino acid sequence having at least 80%, at least 85%, at least 90%, at least 95%, at least 99% or 100% identity to SEQ ID NO:30; a CDR2 comprising an amino acid sequence having at least 80%, at least 85%, at least 90%, at least 95%, at least 99% or 100% identity to SEQ ID NO:31 (having the sequence of DAS); and a CDR3 comprising an amino acid sequence having at least 80%, at least 85%, at least 90%, at least 95%, at least 99% or 100% identity to SEQ ID NO:32.

In some aspects, VH1 comprises a CDR1 comprising an amino acid sequence having at least 80%, at least 85%, at least 90%, at least 95%, at least 99% or 100% identity to SEQ ID NO:10; a CDR2 comprising an amino acid sequence having at least 80%, at least 85%, at least 90%, at least 95%, at least 99% or 100% identity to SEQ ID NO:11; and a CDR3 comprising an amino acid sequence having at least 80%, at least 85%, at least 90%, at least 95%, at least 99% or 100% identity to SEQ ID NO:12; VH2 comprises a CDR1 comprising an amino acid sequence having at least 80%, at least 85%, at least 90%, at least 95%, at least 99% or 100% identity to SEQ ID NO:18; a CDR2 comprising an amino acid sequence having at least 80%, at least 85%, at least 90%, at least 95%, at least 99% or 100% identity to SEQ ID NO:19; and a CDR3 comprising an amino acid sequence having at least 80%, at least 85%, at least 90%, at least 95%, at least 99% or 100% identity to SEQ ID NO:20; VH3 comprises a CDR1 comprising an amino acid sequence having at least 80%, at least 85%, at least 90%, at least 95%, at least 99% or 100% identity to SEQ ID NO:26; a CDR2 comprising an amino acid sequence having at least 80%, at least 85%, at least 90%, at least 95%, at least 99% or 100% identity to SEQ ID NO:27; and a CDR3 comprising an amino acid sequence having at least 80%, at least 85%, at least 90%, at least 95%, at least 99% or 100% identity to SEQ ID NO:28; VL1 comprises a CDR1 comprising an amino acid sequence having at least 80%, at least 85%, at least 90%, at least 95%, at least 99% or 100% identity to SEQ ID NO:14; a CDR2 comprising an amino acid sequence having at least 80%, at least 85%, at least 90%, at least 95%, at least 99% or 100% identity to SEQ ID NO:15 (having the sequence of GTN); and a CDR3 comprising an amino acid sequence having at least 80%, at least 85%, at least 90%, at least 95%, at least 99% or 100% identity to SEQ ID NO:16; VL2 comprises a CDR1 comprising an amino acid sequence having at least 80%, at least 85%, at least 90%, at least 95%, at least 99% or 100% identity to SEQ ID NO:22; a CDR2 comprising an amino acid sequence having at least 80%, at least 85%, at least 90%, at least 95%, at least 99% or 100% identity to SEQ ID NO:23 (having the sequence of GAS); and a CDR3 comprising an amino acid sequence having at least 80%, at least 85%, at least 90%, at least 95%, at least 99% or 100% identity to SEQ ID NO:24; and VL3 comprises a CDR1 comprising an amino acid sequence having at least 80%, at least 85%, at least 90%, at least 95%, at least 99% or 100% identity to SEQ ID NO:30; a CDR2 comprising an amino acid sequence having at least 80%, at least 85%, at least 90%, at least 95%, at least 99% or 100% identity to SEQ ID NO:31 (having the sequence of DAS); and a CDR3 comprising an amino acid sequence having at least 80%, at least 85%, at least 90%, at least 95%, at least 99% or 100% identity to SEQ ID NO:32.

In some aspects, VH1 comprises a CDR1 comprising an amino acid sequence having at least 80%, at least 85%, at least 90%, at least 95%, at least 99% or 100% identity to SEQ ID NO:10; a CDR2 comprising an amino acid sequence having at least 80%, at least 85%, at least 90%, at least 95%, at least 99% or 100% identity to SEQ ID NO:11; and a CDR3 comprising an amino acid sequence having at least 80%, at least 85%, at least 90%, at least 95%, at least 99% or 100% identity to SEQ ID NO:12; VH2 comprises a CDR1 comprising an amino acid sequence having at least 80%, at least 85%, at least 90%, at least 95%, at least 99% or 100% identity to SEQ ID NO:26; a CDR2 comprising an amino acid sequence having at least 80%, at least 85%, at least 90%, at least 95%, at least 99% or 100% identity to SEQ ID NO:27; and a CDR3 comprising an amino acid sequence having at least 80%, at least 85%, at least 90%, at least 95%, at least 99% or 100% identity to SEQ ID NO:28; VH3 comprises a CDR1 comprising an amino acid sequence having at least 80%, at least 85%, at least 90%, at least 95%, at least 99% or 100% identity to SEQ ID NO:18; a CDR2 comprising an amino acid sequence having at least 80%, at least 85%, at least 90%, at least 95%, at least 99% or 100% identity to SEQ ID NO:19; and a CDR3 comprising an amino acid sequence having at least 80%, at least 85%, at least 90%, at least 95%, at least 99% or 100% identity to SEQ ID NO:20; VL1 comprises a CDR1 comprising an amino acid sequence having at least 80%, at least 85%, at least 90%, at least 95%, at least 99% or 100% identity to SEQ ID NO:14; a CDR2 comprising an amino acid sequence having at least 80%, at least 85%, at least 90%, at least 95%, at least 99% or 100% identity to SEQ ID NO:15 (having the sequence of GTN); and a CDR3 comprising an amino acid sequence having at least 80%, at least 85%, at least 90%, at least 95%, at least 99% or 100% identity to SEQ ID NO:16; VL2 comprises a CDR1 comprising an amino acid sequence having at least 80%, at least 85%, at least 90%, at least 95%, at least 99% or 100% identity to SEQ ID NO:30; a CDR2 comprising an amino acid sequence having at least 80%, at least 85%, at least 90%, at least 95%, at least 99% or 100% identity to SEQ ID NO:31 (having the sequence of DAS); and a CDR3 comprising an amino acid sequence having at least 80%, at least 85%, at least 90%, at least 95%, at least 99% or 100% identity to SEQ ID NO:32; and VL3 comprises a CDR1 comprising an amino acid sequence having at least 80%, at least 85%, at least 90%, at least 95%, at least 99% or 100% identity to SEQ ID NO:22; a CDR2 comprising an amino acid sequence having at least 80%, at least 85%, at least 90%, at least 95%, at least 99% or 100% identity to SEQ ID NO:23 (having the sequence of GAS); and a CDR3 comprising an amino acid sequence having at least 80%, at least 85%, at least 90%, at least 95%, at least 99% or 100% identity to SEQ ID NO:24.

In some aspects, VH1 comprises a CDR1 comprising an amino acid sequence having at least 80%, at least 85%, at least 90%, at least 95%, at least 99% or 100% identity to SEQ ID NO:26; a CDR2 comprising an amino acid sequence having at least 80%, at least 85%, at least 90%, at least 95%, at least 99% or 100% identity to SEQ ID NO:27; and a CDR3 comprising an amino acid sequence having at least 80%, at least 85%, at least 90%, at least 95%, at least 99% or 100% identity to SEQ ID NO:28; VH2 comprises a CDR1 comprising an amino acid sequence having at least 80%, at least 85%, at least 90%, at least 95%, at least 99% or 100% identity to SEQ ID NO:10; a CDR2 comprising an amino acid sequence having at least 80%, at least 85%, at least 90%, at least 95%, at least 99% or 100% identity to SEQ ID NO:11; and a CDR3 comprising an amino acid sequence having at least 80%, at least 85%, at least 90%, at least 95%, at least 99% or 100% identity to SEQ ID NO:12; VH3 comprises a CDR1 comprising an amino acid sequence having at least 80%, at least 85%, at least 90%, at least 95%, at least 99% or 100% identity to SEQ ID NO:18; a CDR2 comprising an amino acid sequence having at least 80%, at least 85%, at least 90%, at least 95%, at least 99% or 100% identity to SEQ ID NO:19; and a CDR3 comprising an amino acid sequence having at least 80%, at least 85%, at least 90%, at least 95%, at least 99% or 100% identity to SEQ ID NO:20; VL1 comprises a CDR1 comprising an amino acid sequence having at least 80%, at least 85%, at least 90%, at least 95%, at least 99% or 100% identity to SEQ ID NO:30; a CDR2 comprising an amino acid sequence having at least 80%, at least 85%, at least 90%, at least 95%, at least 99% or 100% identity to SEQ ID NO:31 (having the sequence of DAS); and a CDR3 comprising an amino acid sequence having at least 80%, at least 85%, at least 90%, at least 95%, at least 99% or 100% identity to SEQ ID NO:32; VL2 comprises a CDR1 comprising an amino acid sequence having at least 80%, at least 85%, at least 90%, at least 95%, at least 99% or 100% identity to SEQ ID NO:14; a CDR2 comprising an amino acid sequence having at least 80%, at least 85%, at least 90%, at least 95%, at least 99% or 100% identity to SEQ ID NO:15 (having the sequence of GTN); and a CDR3 comprising an amino acid sequence having at least 80%, at least 85%, at least 90%, at least 95%, at least 99% or 100% identity to SEQ ID NO:16; and VL3 comprises a CDR1 comprising an amino acid sequence having at least 80%, at least 85%, at least 90%, at least 95%, at least 99% or 100% identity to SEQ ID NO:22; a CDR2 comprising an amino acid sequence having at least 80%, at least 85%, at least 90%, at least 95%, at least 99% or 100% identity to SEQ ID NO:23 (having the sequence of GAS); and a CDR3 comprising an amino acid sequence having at least 80%, at least 85%, at least 90%, at least 95%, at least 99% or 100% identity to SEQ ID NO:24.

In some aspects, VH1 comprises a CDR1 comprising an amino acid sequence having at least 80%, at least 85%, at least 90%, at least 95%, at least 99% or 100% identity to SEQ ID NO:26; a CDR2 comprising an amino acid sequence having at least 80%, at least 85%, at least 90%, at least 95%, at least 99% or 100% identity to SEQ ID NO:27; and a CDR3 comprising an amino acid sequence having at least 80%, at least 85%, at least 90%, at least 95%, at least 99% or 100% identity to SEQ ID NO:28; VH2 comprises a CDR1 comprising an amino acid sequence having at least 80%, at least 85%, at least 90%, at least 95%, at least 99% or 100% identity to SEQ ID NO:18; a CDR2 comprising an amino acid sequence having at least 80%, at least 85%, at least 90%, at least 95%, at least 99% or 100% identity to SEQ ID NO:19; and a CDR3 comprising an amino acid sequence having at least 80%, at least 85%, at least 90%, at least 95%, at least 99% or 100% identity to SEQ ID NO:20; VH3 comprises a CDR1 comprising an amino acid sequence having at least 80%, at least 85%, at least 90%, at least 95%, at least 99% or 100% identity to SEQ ID NO:10; a CDR2 comprising an amino acid sequence having at least 80%, at least 85%, at least 90%, at least 95%, at least 99% or 100% identity to SEQ ID NO:11; and a CDR3 comprising an amino acid sequence having at least 80%, at least 85%, at least 90%, at least 95%, at least 99% or 100% identity to SEQ ID NO:12; VL1 comprises a CDR1 comprising an amino acid sequence having at least 80%, at least 85%, at least 90%, at least 95%, at least 99% or 100% identity to SEQ ID NO:30; a CDR2 comprising an amino acid sequence having at least 80%, at least 85%, at least 90%, at least 95%, at least 99% or 100% identity to SEQ ID NO:31 (having the sequence of DAS); and a CDR3 comprising an amino acid sequence having at least 80%, at least 85%, at least 90%, at least 95%, at least 99% or 100% identity to SEQ ID NO:32; VL2 comprises a CDR1 comprising an amino acid sequence having at least 80%, at least 85%, at least 90%, at least 95%, at least 99% or 100% identity to SEQ ID NO:22; a CDR2 comprising an amino acid sequence having at least 80%, at least 85%, at least 90%, at least 95%, at least 99% or 100% identity to SEQ ID NO:23 (having the sequence of GAS); and a CDR3 comprising an amino acid sequence having at least 80%, at least 85%, at least 90%, at least 95%, at least 99% or 100% identity to SEQ ID NO:24; and VL3 comprises a CDR1 comprising an amino acid sequence having at least 80%, at least 85%, at least 90%, at least 95%, at least 99% or 100% identity to SEQ ID NO:14; a CDR2 comprising an amino acid sequence having at least 80%, at least 85%, at least 90%, at least 95%, at least 99% or 100% identity to SEQ ID NO:15 (having the sequence of GTN); and a CDR3 comprising an amino acid sequence having at least 80%, at least 85%, at least 90%, at least 95%, at least 99% or 100% identity to SEQ ID NO:16.

In some aspects, VH1, VH2 and VH3 each independently comprise an amino acid sequence having at least 80%, at least 85%, at least 90%, at least 95%, at least 99% or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359. In some aspects, the sequence has a mutation at N62, for example, N62Q or N62Q at, for example, SEQ ID NO:175.

In some aspects, VL1, VL2 and VL3 each independently comprise an amino acid sequence having at least 80%, at least 85%, at least 90%, at least 95%, or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346.

In some aspects, VH1, VH2 and VH3 each comprise an amino acid sequence having at least 80%, at least 85%, at least 90%, at least 95%, at least 99% or 100% identity to SEQ ID NO:17; and VL1, VL2 and VL3 each comprise an amino acid sequence having at least 80%, at least 85%, at least 90%, at least 95%, at least 99% or 100% identity to SEQ ID NO:21.

In some aspects, VH1 and VH3 each comprise an amino acid sequence having at least 80%, at least 85%, at least 90%, at least 95%, at least 99% or 100% identity to SEQ ID NO:17; VH2 comprises an amino acid sequence having at least 80%, at least 85%, at least 90%, at least 95%, at least 99% or 100% identity to SEQ ID NO:25; VL1 and VL3 each comprise an amino acid sequence having at least 80%, at least 85%, at least 90%, at least 95%, at least 99% or 100% identity to SEQ ID NO:21; and VL2 comprises an amino acid sequence having at least 80%, at least 85%, at least 90%, at least 95%, at least 99% or 100% identity to SEQ ID NO:29.

In some aspects, VH1 and VH2 each comprise an amino acid sequence having at least 80%, at least 85%, at least 90%, at least 95%, at least 99% or 100% identity to SEQ ID NO:17; VH3 comprises an amino acid sequence having at least 80%, at least 85%, at least 90%, at least 95%, at least 99% or 100% identity to SEQ ID NO:25; VL1 and VL2 each comprise an amino acid sequence having at least 80%, at least 85%, at least 90%, at least 95%, at least 99% or 100% identity to SEQ ID NO:21; and VL3 comprises an amino acid sequence having at least 80%, at least 85%, at least 90%, at least 95%, at least 99% or 100% identity to SEQ ID NO:29.

In some aspects, VH2 and VH3 each comprise an amino acid sequence having at least 80%, at least 85%, at least 90%, at least 95%, at least 99% or 100% identity to SEQ ID NO:17; VH1 comprises an amino acid sequence having at least 80%, at least 85%, at least 90%, at least 95%, at least 99% or 100% identity to SEQ ID NO:25; VL2 and VL3 each comprise an amino acid sequence having at least 80%, at least 85%, at least 90%, at least 95%, at least 99% or 100% identity to SEQ ID NO:21; and VL1 comprises an amino acid sequence having at least 80%, at least 85%, at least 90%, at least 95%, at least 99% or 100% identity to SEQ ID NO:29.

In some aspects, VH1 comprises an amino acid sequence having at least 80%, at least 85%, at least 90%, at least 95%, at least 99% or 100% identity to SEQ ID NO:9; VH2 comprises an amino acid sequence having at least 80%, at least 85%, at least 90%, at least 95%, at least 99% or 100% identity to SEQ ID NO:17; VH3 comprises an amino acid sequence having at least 80%, at least 85%, at least 90%, at least 95%, at least 99% or 100% identity to SEQ ID NO:25; VL1 comprises an amino acid sequence having at least 80%, at least 85%, at least 90%, at least 95%, at least 99% or 100% identity to SEQ ID NO:13; VL2 comprises an amino acid sequence having at least 80%, at least 85%, at least 90%, at least 95%, at least 99% or 100% identity to SEQ ID NO:21; and VL3 comprises an amino acid sequence having at least 80%, at least 85%, at least 90%, at least 95%, at least 99% or 100% identity to SEQ ID NO:29.

In some aspects, VH1 comprises an amino acid sequence having at least 80%, at least 85%, at least 90%, at least 95%, at least 99% or 100% identity to SEQ ID NO:9; VH2 comprises an amino acid sequence having at least 80%, at least 85%, at least 90%, at least 95%, at least 99% or 100% identity to SEQ ID NO:25; VH3 comprises an amino acid sequence having at least 80%, at least 85%, at least 90%, at least 95%, at least 99% or 100% identity to SEQ ID NO:17; VL1 comprises an amino acid sequence having at least 80%, at least 85%, at least 90%, at least 95%, at least 99% or 100% identity to SEQ ID NO:13; VL2 comprises an amino acid sequence having at least 80%, at least 85%, at least 90%, at least 95%, at least 99% or 100% identity to SEQ ID NO:29; and VL3 comprises an amino acid sequence having at least 80%, at least 85%, at least 90%, at least 95%, at least 99% or 100% identity to SEQ ID NO:21.

In some aspects, VH1 comprises an amino acid sequence having at least 80%, at least 85%, at least 90%, at least 95%, at least 99% or 100% identity to SEQ ID NO:25; VH2 comprises an amino acid sequence having at least 80%, at least 85%, at least 90%, at least 95%, at least 99% or 100% identity to SEQ ID NO:9; VH3 comprises an amino acid sequence having at least 80%, at least 85%, at least 90%, at least 95%, at least 99% or 100% identity to SEQ ID NO:17; VL1 comprises an amino acid sequence having at least 80%, at least 85%, at least 90%, at least 95%, at least 99% or 100% identity to SEQ ID NO:29; VL2 comprises an amino acid sequence having at least 80%, at least 85%, at least 90%, at least 95%, at least 99% or 100% identity to SEQ ID NO:13; and VL3 comprises an amino acid sequence having at least 80%, at least 85%, at least 90%, at least 95%, at least 99% or 100% identity to SEQ ID NO:21.

In some aspects, VH1 comprises an amino acid sequence having at least 80%, at least 85%, at least 90%, at least 95%, at least 99% or 100% identity to SEQ ID NO:25; VH2 comprises an amino acid sequence having at least 80%, at least 85%, at least 90%, at least 95%, at least 99% or 100% identity to SEQ ID NO:17; VH3 comprises an amino acid sequence having at least 80%, at least 85%, at least 90%, at least 95%, at least 99% or 100% identity to SEQ ID NO:9; VL1 comprises an amino acid sequence having at least 80%, at least 85%, at least 90%, at least 95%, at least 99% or 100% identity to SEQ ID NO:29; VL2 comprises an amino acid sequence having at least 80%, at least 85%, at least 90%, at least 95%, at least 99% or 100% identity to SEQ ID NO:21; and VL3 comprises an amino acid sequence having at least 80%, at least 85%, at least 90%, at least 95%, at least 99% or 100% identity to SEQ ID NO:13.

In some aspects, the immunoglobulin hinge comprises an upper hinge region, a middle hinge region, a lower hinge region, or a combination thereof.

In some aspects, linkers L1-L8 each independently have a length of from about 0 amino acids to about 50 amino acids.

In some aspects, linkers L1-L8 each independently comprise an amino acid sequence having at least 80%, at least 85%, at least 90%, at least 95%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG).

In some aspects, one or more of linkers L1-L8 are non-immunogenic.

In some aspects, one or more of linkers L1-L8 do not contain a consensus T cell epitope.

In some aspects, the Fc region comprises at least one knob-into-hole modification or Fc effector function knockout mutation.

In some aspects, the antigen binding polypeptide complex is an IgG1 or IgG4 antibody and the knob-into-hole modification comprises (i) knob substitutions of S354C and T366W and hole substitutions of Y349C, T366S, L368A and Y407V; (ii) hole substitutions of M428L and N434S or N434A; (iii) hole substitutions of M252Y, S254T and T256E; or (iv) a combination thereof, based on the EU numbering scheme. In some aspects, the hole substitutions are M428L and N434S, based on the EU numbering scheme. In some aspects, the hole substitution is N434A, based on the EU numbering scheme.

In some aspects, the antigen binding polypeptide complex is an IgG1 or IgG4 antibody and the Fc effector function knockout mutation is L234A, L235A, P239A or a combination thereof, based on the EU numbering scheme.

In some aspects, the antigen binding polypeptide complex comprises a detectable label. In some aspects, the detectable label is a radioactive label, chemiluminescent label, fluorescent label, enzyme, peptide tag, or a combination thereof. In some aspects, the peptide tag is a polyhistidine tag consisting of from about four to about 10 histidine residues. In some aspects, the polyhistidine tag consists of about eight histidine residues.

In some aspects, the antigen binding polypeptide complex specifically binds to a SARS-CoV-2 protein with an equilibrium dissociation constant (K_{D}) of from about 10 μM to about 1 μM.

In some aspects, a variable region of an antibody may require site specific deglycosylation. In some aspects, and with respect to E8 VH mAb, an N62 mutation is made to remove a high mannose glycosylate residue or moiety. Preferred mutations include, but are not limited to, N62Q or N62S.

In some aspects, the SARS-CoV-2 virus is the original Wuhan strain (WA1), D614G spike protein mutant (D614G), Alpha variant (B.1.1.7), Beta variant (B.1.351), Gamma variant (P.1), Kappa variant (B.1.617.1), Delta variant (B.1.617.2 or AY.1), Epsilon variant (B.1.427), Iota variant (B.1.526), Lambda variant (C.37), Mu variant (B.1.621), C.1.2 variant, Omicron variant (BA1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, or CH.1.1), or a combination thereof.

In some aspects, the SARS-CoV-2 protein is a membrane protein, nucleocapsid protein, envelope protein, or spike protein. In some aspects, the SARS-CoV-2 protein is a spike protein.

Also provided herein is an antibody or antigen binding fragment thereof comprising an antigen binding polypeptide complex provided herein. In some aspects, the antibody is IgG, IgM, IgE, IgA or IgD. In some aspects, the IgG is IgG1, IgG2, IgG3 or IgG4. In some aspects, the antigen binding fragment is a Fab, scFab, Fab′, F(ab′)₂, Fv or scFv. In some aspects, the antibody is human or humanized. In some aspects, the antibody is a monoclonal antibody, grafted antibody, or chimeric antibody.

Also provided herein is an anti-SARS-CoV2 multivalent antibody that exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody.

Also provided herein is a pharmaceutical composition comprising an antigen binding polypeptide, antigen binding polypeptide complex, antibody or antigen binding fragment thereof provided herein. In some aspects, the pharmaceutical composition comprises (1) one or more polynucleotides encoding an antigen binding polypeptide or antigen binding polypeptide complex and (2) a carrier. In some aspects, the pharmaceutical composition comprises (1) one or more polynucleotides encoding an antigen binding polypeptide or antigen binding polypeptide complex (e.g., antibody or antigen binding fragment thereof) provided herein, and (2) a carrier selected from the group consisting of a lipid or a lipid nanoparticle. In some aspects, the one or more polynucleotides encoding an antigen binding polypeptide or antigen binding polypeptide complex is a stabilized polynucleotide resistant to degradation or decomposition in vivo. In some aspects, the stabilized polynucleotide is capped at one end (5′ cap) and has a long polyadenylated (polyA) tail at the other end to form a stabilized mRNA having 5′ and 3′ UTRs. In some aspects, the polyA tail is about 50 bp, about 100 bp, about 120 bp, or about 150 bp. In some aspects, modified nucleosides may be incorporated into the mRNA to increase translation and/or to lower potential immunogenicity.

Also provided herein is a pharmaceutical composition comprising an antigen binding polypeptide complex, antibody, or antigen binding fragment thereof provided herein, and an additional pharmaceutical agent.

Also provided herein is a kit comprising an antigen binding polypeptide complex, antibody or antigen binding fragment thereof, or pharmaceutical composition provided herein.

Also provided herein is a method of preventing or treating a SARS-CoV-2 viral infection in a subject in need thereof, comprising administering to the subject an effective amount of an antigen binding polypeptide or polypeptide complex provided herein, an antibody or antigen binding fragment thereof provided herein, a pharmaceutical composition provided herein, or a combination thereof. Delivery of the therapeutic antigen binding polypeptide or antigen binding polypeptide complex can be by direct administration of the therapeutic or by an indirect method that is inclusive of a modified mRNA composition that is administered to the patient in need of treatment thereof by a therapeutic antigen binding polypeptide or antigen binding polypeptide complex translated from the modified mRNA that codes for the therapeutic polypeptide or polypeptide complex.

Also provided herein is a method of preventing or treating coronavirus disease 2019 (COVID-19) in a subject in need thereof, comprising administering to the subject an effective amount of an antigen binding polypeptide or polypeptide complex provided herein, an antibody or antigen binding fragment thereof provided herein, a pharmaceutical composition provided herein, or a combination thereof.

Also provided herein is a method of diagnosing a subject as being infected with a SARS-CoV-2 virus or suspected of being infected with a SARS-CoV-2 virus, comprising (i) contacting a sample obtained from the subject with an antigen binding polypeptide or polypeptide complex provided herein or an antibody or antigen binding fragment thereof provided herein; (ii) detecting the presence or absence of a virus complex which contains the polypeptide complex, antibody or fragment and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected.

Also provided herein is a method of diagnosing a subject as not being infected with a SARS-CoV-2 virus or not suspected of being infected with a SARS-CoV-2 virus, comprising (i) contacting a sample obtained from the subject with an antigen binding polypeptide or polypeptide complex provided herein or an antibody or antigen binding fragment thereof provided herein; (ii) detecting the presence or absence of a virus complex which contains the polypeptide complex, antibody or fragment and a SARS-CoV-2 virus, virion or fragment thereof, and (iii) diagnosing the subject as not being infected with a SARS-CoV-2 virus or not suspected of being infected with a SARS-CoV-2 virus when the presence of the virus complex is not detected.

Also provided herein is a method of diagnosing a subject as having COVID-19 or suspected of having COVID-19, comprising (i) contacting a sample obtained from the subject with an antigen binding polypeptide or polypeptide complex provided herein or an antibody or antigen binding fragment thereof provided herein; (ii) detecting the presence or absence of a virus complex which contains the polypeptide complex, antibody or fragment and a SARS-CoV-2 virus, virion or fragment thereof, and (iii) diagnosing the subject as having COVID-19 or suspected of having COVID-19 when the presence of the virus complex is detected.

Also provided herein is a method of diagnosing a subject as not having COVID-19 or not suspected of having COVID-19, comprising (i) contacting a sample obtained from the subject with an antigen binding polypeptide or polypeptide complex provided herein or an antibody or antigen binding fragment thereof provided herein; (ii) detecting the presence or absence of a virus complex which contains the polypeptide complex, antibody or fragment and a SARS-CoV-2 virus, virion or fragment thereof, and (iii) diagnosing the subject as not having COVID-19 or not suspected of having COVID-19 when the presence of the virus complex is not detected.

In some aspects, the sample is a nasal swab, tissue sample, saliva, plasma or blood.

In some aspects, detecting the presence or absence of the virus complex comprises an enzyme linked immunosorbent assay (ELISA), immunospot assay, lateral flow assay, flow cytometry, immunohistochemistry or western blot.

In some aspects, the SARS-CoV-2 virus is the original Wuhan strain (WA1), D614G spike protein mutant (D614G), Alpha variant (B.1.1.7), Beta variant (B.1.351), Gamma variant (P.1), Kappa variant (B.1.617.1), Delta variant (B.1.617.2 or AY.1), Epsilon variant (B.1.427), Iota variant (B.1.526), Lambda variant (C.37), Mu variant (B.1.621), C.1.2 variant, Omicron variant (BA1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, or CH.1.1), or a combination thereof.

In some aspects, the SARS-CoV-2 virus is the original Wuhan strain (WA1), D614G spike protein mutant (D614G), Alpha variant (B.1.1.7), Beta variant (B.1.351), Gamma variant (P.1), Kappa variant (B.1.617.1), Delta variant (B.1.617.2 or AY.1), Epsilon variant (B.1.427), Iota variant (B.1.526), Lambda variant (C.37), Mu variant (B.1.621), C.1.2 variant, Omicron variant (BA1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, or CH.1.1), or a combination thereof.

Also provided herein is a method of preventing immune escape of a SARS-CoV2 virus in a subject infected with the SARS-CoV2 virus, comprising administering to the subject an effective amount of an antigen binding polypeptide or polypeptide complex provided herein, an antibody or antigen binding fragment thereof provided herein, a pharmaceutical composition provided herein, or a combination thereof.

Also provided herein is a method of preventing immune escape of a SARS-CoV2 virus in a subject infected with the SARS-CoV2 virus, comprising administering to the subject an effective amount of an anti-SARS-CoV2 multivalent antibody that exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody.

Also provided herein is a method of neutralizing a SARS-CoV-2 virus in a subject infected with the SARS-CoV2 virus, comprising administering to the subject an effective amount of an antigen binding polypeptide or polypeptide complex provided herein, an antibody or antigen binding fragment thereof provided herein, or a combination thereof.

Also provided herein is a method of increasing neutralization potency against a SARS-CoV-2 virus in a subject infected with the SARS-CoV2 virus, comprising administering to the subject an effective amount of an antigen binding polypeptide or polypeptide complex provided herein, an antibody or antigen binding fragment thereof provided herein, or a combination thereof.

Also provided herein is a method of increasing neutralization potency against a SARS-CoV-2 virus in a subject infected with the SARS-CoV2 virus, comprising administering to the subject an effective amount of an anti-SARS-CoV2 multivalent antibody that exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1 shows an exemplary configuration of a monospecific, trivalent cross-over dual variable (CODV) immunoglobulin antibody made utilizing variable fragments (Fv) from the anti-SARS-CoV-2 antibody, B1-182.1 (Fv182.1, dotted). The Fc region contains a knob-into-hole modification to increase yield and correct association of heavy chains.

FIG. 2A-2C shows exemplary configurations of bispecific, trivalent CODV immunoglobulin antibodies made utilizing variable fragments (Fv) from B1-182.1 (Fv182.1, dotted) and the anti-SARS-CoV-2 antibody, A19-61.1 (Fv61.1, grid). The Fc region contains a knob-into-hole modification to increase yield and correct association of heavy chains.

FIG. 3A-3D shows exemplary configurations of trispecific, trivalent CODV immunoglobulin antibodies made utilizing variable fragments (Fv) from B1-182.1 (“182.1” or “Fv182 Class I”, dotted), the anti-SARS-CoV-2 antibody, A19-46.1 (“46.1” or “Fv46 Class II”, striped) and A19-61.1 (“61.1” or “Fv61 Class III”, grid). The Fc region contains a knob-into-hole modification to increase yield and correct association of heavy chains.

FIG. 4A shows purity of CODV immunoglobulin antibodies evaluated under non-reducing (nr) and reducing (r) conditions on a Coomassie SDS-PAGE gel (representative gels shown). A molecular weight marker standard (MW) is also shown.

FIG. 4B shows CODV immunoglobulin bispecific and trispecific antibody traces shown before (top row) and after size exclusion chromatography (SEC) (bottom row). The dotted box indicates the fractions combined to make final preparations of the indicated multispecific antibodies. The bottom row shows analytic SEC traces for the purified multispecific antibodies.

FIG. 5A shows cross-over dual variable immunoglobulin antibody designs used in FIGS. 5B-5E and Table 4.

FIG. 5B shows the expected binding of Fv182.1 (“Fv182”), Fv46.1 (“Fv46”), and Fv61.1 (“Fv61”) to viral spike receptor-binding domain (RBD) proteins containing the indicated Fv isolating mutations, that were selected to knockout binding a single component Fv while leaving binding of the remain Fv components unchanged.

FIG. 5C-5E shows validation of multispecific Fv binding activity using Fv isolating mutations in RBD and enzyme-linked immunosorbent assay (ELISA). Shown is ELISA binding for parental component antibody controls (FIG. 5C, B1-182.1, A19-46.1, and A19-61.1), bispecific antibodies (FIG. 5D, 182.1/182.1-61.1, 182.1/61.1-182.1, 61.1/182.1-182.1, 61.1/61.1-182.1 and 61.1/182.1-61.1), and trispecific antibodies (FIG. 5E, 46.1/61.1-182.1, 46.1/182.1-61.1, 61.1/46.1-182.1 and 61.1/182.1-46.1) against WA-1 RBD (wt) or RBD proteins containing one or more Fv-specific mutation shown and chosen to interrogate binding of one Fv component at a time in each multispecific antibody.

FIG. 6A shows a schematic illustration of arrangement of antibody variable domains in different CODVs. The domains are dotted, striped, grid, and gray for Fv182.1 (182.1), Fv46.1 (46.1), Fv61.1 (61.1) and constant domains, respectively.

FIG. 6B shows combinations of RBD mutations on the SARS-CoV-2 spike designed to distinguish different binding modes of CODV.

FIG. 6C is a schematic of CODV 61.1-182.1 (left) and binding of the CODV 61.1-182.1 to spike protein mutants (middle left and right). For clarity, the Fv domain that is not able to bind to the mutant is colored black. CODV binding to K444E/L452R mutant was observed, indicating CODV 61.1-182.1 can bind to spike protein through Fv182 domain (middle left). However, no CODV was observed to bind the L452R/F486S mutant (middle right), suggesting that the CODV 61.1-182.1 cannot bind through the Fv61 domain in the trimer context. A model of the CODV 61.1-182.1 bound to the SARS-CoV-2 spike protein is shown in the allowed Fv182-binding mode (right). For clarity, only one CODV is shown to bind to the spike protein in RBD-up conformation. The white scale bar represents 10 nm.

FIG. 6D is a schematic of CODV 182.1-61.1 (left) and binding of the CODV 182.1-61.1 to spike protein mutants (middle left and right). For clarity, the Fv domain that is not able to bind to the mutant is colored black. CODV was observed to bind both K444E/L452R and L452R/F486S mutants, indicating the CODV 182.1-61.1 can bind to spike protein through either Fv182 domain (middle left) or Fv61 domain (middle right). Models of the CODV 182.1-61.1 bound to the SARS-CoV-2 spike protein are shown in both Fv182.1-binding and Fv61.1-binding modes (right). The scale is the same as in FIG. 6C.

FIG. 6E is a schematic of CODV 46.1-182.1 (left) and binding of the CODV 46.1-182.1 to spike protein mutants (middle left and right). For clarity, the Fv domain that is not able to bind to the mutant is colored black. CODV was observed to bind both K444E/L452R and K444E/F486S mutants, indicating the CODV 46.1-182.1 can bind to the spike protein through both Fv182 and Fv46 domains (middle left and right). Models of the CODV 46.1-182.1 bound to the SARS-CoV-2 spike protein are shown to bind through Fv182 and Fv46-binding modes (right). The scale is the same as in FIG. 6C.

FIG. 6F is a schematic of CODV 182.1-46.1 (left) and binding of the CODV 182.1-46.1 to spike protein mutants (middle left and right). For clarity, the Fv domain that is not able to bind to the mutant is colored black. CODV was observed to bind K444E/L452R, indicating the CODV 182.1-46.1 can bind to the spike protein through the Fv182.1 domain (middle left). However, no CODV was observed to bind the K444E/F486S mutant (middle right), suggesting that the CODV 182.1-46.1 cannot bind through the Fv46 domain in the trimer context. Model of the CODV 182.1-46.1 bound to the SARS-CoV-2 spike protein is shown to bind through the allowed Fv182 domain (right). The scale is the same as in FIG. 6C.

FIG. 6G shows CODV 46.1-182.1- and 182.1-46.1-induced aggregation of the Omicron spike protein using negative stain-electron microscopy (NSEM).

FIG. 6H shows the spike-aggregating potential of 61.1/46.1-182.1 using NSEM.

FIG. 7 shows SARS-CoV-2 escape potential by single antibodies, combinations of single antibodies, and candidate multispecific antibodies.

FIG. 8 shows results from three independent experiments showing SARS-CoV-2 escape potential of T3_V3 (having 61.1, 46.1 and 182.1 binding regions), compared to an antibody having binding regions for 182.1, 61.1 and 46.1 (182.1+61.1+46.1); an antibody having binding regions for 182.1 (182.1); bebtelovimab (LY1404); and an AZD7442 cocktail (2196+2130).

FIG. 9 shows the resulting serum concentration of E8 glycosylation mutant antibodies in g/mL, at days 1, 2, 5, 7, 9, 14, 21, 28 and 35, following infusion of mice at 5 mg antibody/kg of body weight.

### DETAILED DESCRIPTION OF THE INVENTION

Provided herein are antigen binding polypeptides and polypeptide complexes having improved features. In some aspects, the antigen binding polypeptides and polypeptide complexes have broad reactivity against SARS-CoV-2 variants of concern (VOCs), high neutralization potency, and/or resistance to SARS-CoV-2 escape. In some aspects, the antigen binding polypeptides and polypeptide complexes provided herein are easier to manufacture and administer than current therapeutic antibody cocktails. In some aspects, the antigen binding polypeptides and polypeptide complexes treat and/or prevent SARS-CoV-2 infection and/or COVID-19 in immunocompromised individuals who cannot mount an effective immune response through vaccinations.

Various terms relating to aspects of disclosure are used throughout the specification and claims. Such terms are to be given their ordinary meaning in the art, unless otherwise indicated. Other specifically defined terms are to be construed in a manner consistent with the definitions provided herein.

### Definitions

As used herein, the term “SARS-CoV-2” refers to “severe acute respiratory syndrome coronavirus 2.” SARS-CoV-2 is the strain of coronavirus that causes coronavirus disease 2019 (COVID-19).

As used herein, the term “SARS-CoV-2 virus” includes the SARS-CoV-2 virus, virion or fragment thereof.

As used herein, the term “COVID-19” refers to “coronavirus disease 2019.” COVID-19 is caused by the SARS-CoV-2 virus. Symptoms of COVID-19 are variable, but often include fever, cough, headache, fatigue, breathing difficulties, loss of smell, and loss of taste. Symptoms may begin one to fourteen days after exposure to the virus; however, at least a third of people who are infected do not develop noticeable symptoms. Of those people who develop symptoms noticeable enough to be classed as patients, most develop mild to moderate symptoms (up to mild pneumonia), with approximately 15% developing severe symptoms (dyspnea, hypoxia, or more than 50% lung involvement on imaging), and approximately 5% suffering critical symptoms (respiratory failure, shock, or multiorgan dysfunction). Some people continue to experience a range of effects for months after recovery (“long COVID”).

As used herein, the term “SARS-CoV-2 protein” refers to any protein related to the SARS-CoV-2 virus. The SARS-CoV-2 virus is made of 16 non-structural proteins and four structural proteins. The non-structural proteins are involved mainly in virus replication. The structural proteins include the membrane protein, nucleocapsid protein, envelope protein, and spike protein, which are involved in assembly and infection of the virus. The spike protein has two subunits, S1 and S2, which are responsible for the specific recognition of human angiotensin converting enzyme 2 (hACE2) on the surface of host cells and viral entry. S1 specifically binds the hACE2 through its receptor-binding domain (RBD), whereas S2 facilitates the fusion of the virus to the cell membrane. As such, as used herein, a SARS-CoV-2 protein includes, but is not limited to, a membrane protein, nucleocapsid protein, envelope protein, or spike protein, or a combination thereof. In some aspects, the SARS-CoV-2 protein is a spike protein.

In some aspects, an antigen binding polypeptide or antigen binding polypeptide complex provided herein (e.g., an antibody or antigen binding fragment thereof) is, or is part of, a class I antibody, a class II antibody, or a class III antibody.

As used herein, “class I” refers to an antibody that typically contains a short heavy chain CDR3 region and competes with angiotensin-converting enzyme 2 (ACE2) for binding to the receptor binding domain (RBD) of the spike protein, and only recognizes the “up” configuration of the RBD. See, e.g., Shrestha et al., Front. Immunol. 12:7520003, 2021. As used herein, “class II” refers to an antibody that typically has a long heavy chain CDR3 loop, competes with ACE2 for binding to RBD, and is capable of binding to RBD in both the “up” and “down” configurations. As used herein, “class III” refers to an antibody that is capable of binding to RBD in both the “up” and “down” configurations and typically binds outside of the ACE2 binding region. As used herein, “class IV” refers to an antibody that binds to the highly conserved, cryptic epitope on RBD outside the receptor binding motif (RBM) of the spike protein.

In some aspects, an antigen binding polypeptide or antigen binding polypeptide complex provided herein (e.g., an antibody or antigen binding fragment thereof) belongs to two, three or four classes. Accordingly, in some aspects, an antigen binding polypeptide or antigen binding polypeptide complex provided herein (e.g., an antibody or antigen binding fragment thereof) is a class I and II antibody, class I and III antibody, class I and IV antibody, class II and III antibody, class II and IV antibody, class III and IV antibody, class I, II and III antibody, class I, II and IV antibody, class I, III and IV antibody, class II, III and IV antibody, or class I, II, III and IV antibody.

In some aspects, an antigen binding polypeptide or antigen binding polypeptide complex provided herein comprises one or more class I antigen binding sites, one or more class II antigen binding sites, and/or one or more class III antigen binding sites. In some aspects, VH1 and VL1 and/or VH3 and VL3 are class III antigen binding sites. In some aspects, VH2 and VL2 and/or VH4 and VL4 are class I antigen binding sites. In some aspects, VH1, VL1, VH3 and VL3 are class III antigen binding sites and VH2, VL2, VH4 and VL4 are class I antigen binding sites. In some aspects, VH1 and VL1 are class II antigen binding sites. In some aspects, VH2 and VL2 are class I antigen binding sites. In some aspects, VH3 and VL3 are class I antigen binding sites. In some aspects, VH4 and VL4 are class III antigen binding sites. In some aspects, VH1 and VL1 are class II antigen binding sites, VH2 and VL2 are class I antigen binding sites, VH3 and VL3 are class I antigen binding sites, and VH4 and VL4 are class III antigen binding sites.

As used herein, the term “spike protein” refers to the largest of the four major structural proteins found in the SARS-CoV-2 virus. The spike protein assembles into trimers that form large structures, called spikes or peplomers, that project from the surface of the SARS-CoV-2 virion and mediate entry of the virion into host cells. The SARS-CoV-2 spike protein is also called “spike (S) glycoprotein” and “E2.” The structural and functional properties of this protein have been characterized. See, e.g., Huang et al., Acta Pharmacol. Sin. 41:1141-1149, 2020; Wu et al., Nature 579(7798):265-269, 2020; and Gene ID: 43740568.

Antigen binding sequences (e.g., CDR, VH, VL, heavy chain and light chain sequences from antibodies) for anti-SARS-CoV-2 proteins are known and described, for example, in U.S. Appl. No. 63/147,419. Such sequences include, but are not limited to, SEQ ID NOs:1-40, 81-186 and 188-360.

As used herein, the term “antigen binding polypeptide” refers to a polypeptide having the ability to specifically bind to one or more substances that induce an immune response (i.e., one or more antigens or epitopes of a SARS-CoV-2 protein).

As used herein, the term “antigen binding polypeptide complex” refers to a group of four associated polypeptides having the ability to specifically bind to one or more substances that induce an immune response (e.g., one or more antigens or epitopes of a SARS-CoV-2 protein). An antigen binding polypeptide complex, includes, but is not limited to, an antibody or antigen binding fragment thereof.

The term “antibody” includes, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

The term “monoclonal antibody,” as used herein, refers to an antibody that is produced by a single clone of B-cells and binds to the same epitope. In contrast, the term “polyclonal antibody” refers to a population of antibodies that are produced by different B-cells and bind to different epitopes of the same antigen. The term “antibody” includes, by way of example, monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man.

The antibody can be an antibody that has been altered (e.g., by mutation, deletion, substitution, conjugation to a non-antibody moiety). For example, an antibody can include one or more variant amino acids (compared to a naturally occurring antibody) which change a property (e.g., a functional property) of the antibody. For example, several such alterations are known in the art which affect, e.g., half-life, effector function, and/or immune responses to the antibody in a patient. The term antibody also includes artificial polypeptide constructs which comprise at least one antibody-derived antigen binding site.

An “antigen binding fragment” of an antibody refers to one or more fragments or portions of an antibody that retain the ability to bind specifically to the antigen bound by the whole antibody. It has been shown that the antigen binding function of an antibody can be performed by fragments or portions of a full-length antibody. An antigen binding fragment can contain the antigenic determining regions of an intact antibody (e.g., the complementarity determining regions (CDRs)). Examples of antigen binding fragments of antibodies include, but are not limited to, Fab, Fab′, F(ab′)₂, and Fv fragments, linear antibodies, and single chain antibodies. An antigen binding fragment of an antibody can be derived from any animal species, such as rodents (e.g., mouse, rat, or hamster) and humans or can be artificially produced.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody.

Antigen binding fragments are obtained using conventional techniques known to those with skill in the art, and the fragments screened for utility in the same manner as are intact antibodies. Antigen binding fragments can be produced by recombinant DNA techniques, or by enzymatic or chemical cleavage of intact immunoglobulins.

As used herein, the term “variable region” typically refers to a portion of an antibody, generally, a portion of a light or heavy chain, typically about the amino-terminal 110 to 120 amino acids, or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of an antibody with antigen. In some aspects, the variable region is a mammalian variable region, e.g., a human, mouse or rabbit variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “complementarity determining region” or “CDR”, as used herein, refer to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL”, “VL region,” and “VL domain” are used herein interchangeably to refer to the light chain variable region of an antigen binding polypeptide, antigen binding polypeptide complex, antibody or antigen binding fragment thereof. In some aspects, a VL region is referred to herein as VL1 to denote a first light chain variable region, VL2 to denote a second light chain variable region, VL3 to denote a third light chain variable region, and VL4 to denote a fourth light chain variable region. An enumerated VL region (e.g., VL1) can have the same or different antigen binding properties and/or the same or different sequence as another enumerated VL region (e.g., VL2).

The terms “VH”, “VH region,” and “VH domain” are used herein interchangeably to refer to the heavy chain variable region of an antigen binding polypeptide, antigen binding polypeptide complex, antibody or antigen binding fragment thereof. In some aspects, a VH region is referred to herein as VH1 to denote a first heavy chain variable region, VH2 to denote a second heavy chain variable region, and VH3 to denote a third heavy chain variable region. An enumerated VH region (e.g., VH1) can have the same or different antigen binding properties and/or the same or different sequence as another enumerated VH region (e.g., VH2).

As used herein, “Kabat numbering” and like terms are recognized in the art and refer to a system of numbering amino acid residues in the heavy and light chain variable regions of an antibody or antigen binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the terms “constant region” or “constant domain” are used interchangeably to refer to a portion of an antigen binding polypeptide, antigen binding polypeptide complex, antibody or antigen binding fragment thereof, e.g., a carboxyl terminal portion of a light and/or heavy chain which is not directly involved in binding of an antibody to antigen but which can exhibit various effector functions, such as interaction with the Fc region. The constant region generally has a more conserved amino acid sequence relative to a variable region. In some aspects, an antigen binding polypeptide, antigen binding polypeptide complex, antibody or antigen binding fragment thereof comprises a constant region or portion thereof that is sufficient for antibody-dependent cell-mediated cytotoxicity (ADCC), antibody-dependent cellular phagocytosis (ADCP), and complement-dependent cytotoxicity (CDC). A constant region includes, but is not limited to, a light chain constant region (CL) or heavy chain constant region (CH1, CH2, CH3).

As used herein, the terms “fragment crystallizable region,” “Fc region,” or “Fc domain” are used interchangeably herein to refer to the tail region of an antibody that interacts with cell surface receptors called Fc receptors and some proteins of the complement system. Fc regions typically comprise CH2 and CH3 regions, and, optionally, an immunoglobulin hinge.

As used herein, the terms “immunoglobulin hinge,” “hinge,” “hinge domain” or “hinge region” are used interchangeably to refer to a stretch of heavy chains between the Fab and Fc portions of an antigen binding polypeptide, antigen binding polypeptide complex, antibody or antigen binding fragment thereof. A hinge provides structure, position and flexibility, which assist with normal functioning of antibodies (e.g., for crosslinking two antigens or binding two antigenic determinants on the same antigen molecule). An immunoglobulin hinge is divided into upper, middle and lower hinge regions that can be separated based on structural and/or genetic components. An immunoglobulin hinge of the invention can contain one, two or all three of these regions. Structurally, the upper hinge region stretches from the C terminal end of CH1 to the first hinge disulfide bond. The middle hinge region stretches from the first cysteine to the last cysteine in the hinge. The lower hinge region extends from the last cysteine to the glycine of CH2. The cysteines present in the hinge form interchain disulfide bonds that link the immunoglobulin monomers.

As used herein, the term “Fab” refers to a region of an antibody that binds to an antigen. It is typically composed of one constant and one variable domain of each of the heavy and the light chain.

As used herein, the term “heavy chain” refers to a portion of an antigen binding polypeptide, antigen binding polypeptide complex, antibody or antigen binding fragment thereof typically composed of a heavy chain variable region (VH), a heavy chain constant region 1 (CH1), a heavy chain constant region 2 (CH2), and a heavy chain constant region 3 (CH3). A typical antibody is composed of two heavy chains and two light chains. When used in reference to an antibody, a heavy chain can refer to any distinct type, e.g., alpha (α), delta (δ), epsilon (ε), gamma (γ), and mu (μ), based on the amino acid sequence of the constant region, which gives rise to IgA, IgD, IgE, IgG, and IgM classes of antibodies, respectively, including subclasses of IgG, e.g., IgG1, IgG2, IgG3, and IgG4. Heavy chain amino acid sequences are known in the art. In some aspects, the heavy chain is a human heavy chain.

As used herein, the term “light chain” refers to a portion of an antigen binding polypeptide, antigen binding polypeptide complex, antibody or antigen binding fragment thereof typically composed of a light chain variable region (VL) and a light chain constant region (CL). A typical antibody is composed of two light chains and two heavy chains. When used in reference to an antibody, a light chain can refer to any distinct type, e.g., kappa (κ) or lambda (λ), based on the amino acid sequence of the constant region. Light chain amino acid sequences are known in the art. In some aspects, the light chain is a human light chain.

The term “chimeric” antibody or antigen binding fragment thereof refers to an antibody or antigen binding fragments thereof wherein the amino acid sequence is derived from two or more species. Typically, the variable region of both light and heavy chains corresponds to the variable region of antibodies or antigen binding fragments thereof derived from one species of mammals (e.g., mouse, rat, rabbit, etc.) with the desired specificity, affinity and capability, while the constant regions are homologous to the sequences in antibodies or antigen binding fragments thereof derived from another (usually human) to avoid eliciting an immune response in that species.

The term “humanized” antibody or antigen binding fragment thereof refers to forms of non-human (e.g., murine) antibodies or antigen binding fragments that are specific immunoglobulin chains, chimeric immunoglobulins, or fragments thereof that contain minimal non-human (e.g., murine) sequences. Typically, humanized antibodies or antigen binding fragments thereof are human immunoglobulins in which residues from a complementary determining region (CDR) are replaced by residues from a CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen binding fragment thereof can be further modified by the substitution of additional residues either in the Fv framework region and/or within the replaced non-human residues to refine and optimize antibody or antigen-binding fragment thereof specificity, affinity, and/or capability. In general, a humanized antibody or antigen binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. A humanized antibody or antigen binding fragment thereof can also comprise at least a portion of a constant region, typically that of a human immunoglobulin. Examples of methods used to generate humanized antibodies are known and described, for example, in U.S. Pat. No. 5,225,539; Roguska et al., Proc. Natl. Acad. Sci., USA, 91(3):969-973 (1994), and Roguska et al., Protein Eng. 9(10):895-904 (1996).

The term “human” antibody or antigen binding fragment thereof, as used herein, means an antibody or antigen binding fragment thereof having an amino acid sequence derived from a human immunoglobulin gene locus, where such antibody or antigen binding fragment is made using recombinant techniques known in the art. This definition of a human antibody or antigen binding fragment thereof includes intact or full-length antibodies and fragments thereof.

A polypeptide, polypeptide complex, antibody, antigen binding fragment thereof, polynucleotide, vector or host cell which is “isolated” is a polypeptide, polypeptide complex, antibody, antigen binding fragment thereof, polynucleotide, vector or host cell which is in a form not found in nature. Isolated polypeptides, polypeptide complexes, antibodies, antigen binding fragments thereof, polynucleotides, vectors or host cells include those which have been purified to a degree that they are no longer in a form in which they are found in nature. In some aspects, a polypeptide, polypeptide complex, antibody, antigen binding fragment thereof, polynucleotide, vector or host cell which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

As used herein, an “isoelectric point” or “pI” refer to the pH at which the net charge of a polypeptide (e.g., an antigen binding polypeptide or antigen binding polypeptide complex provided herein) is zero.

As used herein, “pH” refers to a quantitative measure of acidity or basicity. This term translates the values of the concentration of the hydrogen ion-which ordinarily ranges between about 1 and 10⁻¹⁴ gram-equivalents per litre-into numbers between 0 and 14.

As used herein, “SARS-CoV-2 escape” refers to the process by which a SARS-CoV-2 virus acquires one or more mutations that allow the virus to evade the antibodies generated by a particular vaccine.

As used herein, “SARS-CoV-2 evolution” refers to the heritable genetic changes that a SARS-CoV-2 virus accumulates during its life time, which can arise from adaptations in response to environmental changes or the immune response of the host.

The use of the alternative (e.g., “or”) should be understood to mean either one, both, or a combination thereof of the alternatives. As used herein, the indefinite articles “a” or “an” should be understood to refer to “one or more” of any recited or enumerated component.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” “having,” or the like, otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any numerical range, concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined herein are more fully defined by reference to the specification in its entirety.

Various aspects are described in further detail in the following sections.

### Antigen Binding Polypeptides and Polypeptide Complexes

Provided herein are antigen binding polypeptides and polypeptide complexes having certain structural features. These features include a cross-over dual variable (CODV) structure, which has been described, for example, in Int'l Pub. No. WO2017180913A2.

In some aspects, an antigen binding polypeptide complex provided herein comprises a first polypeptide, a second polypeptide, a third polypeptide, and a fourth polypeptide; wherein the first polypeptide has a structure represented by VL1-L1-CL; wherein the second polypeptide has a structure represented by VH1-L2-CH1; wherein the third polypeptide has a structure represented by VH2-L3-VH3-L4-CH1; wherein the fourth polypeptide has a structure represented by VL3-L5-VL2-L6-CL; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; CH1 is an immunoglobulin heavy chain constant region 1; CL is an immunoglobulin light chain constant region; and L1-L6 are amino acid linkers.

In some aspects, an antigen binding polypeptide complex provided herein comprises a first polypeptide, a second polypeptide, a third polypeptide, and a fourth polypeptide; wherein the first polypeptide has a structure represented by VL1-L1-CL; wherein the second polypeptide has a structure represented by VH1-L2-CH1-L3-Fc; wherein the third polypeptide has a structure represented by VH2-L4-VH3-L5-CH1-L6-Fc; wherein the fourth polypeptide has a structure represented by VL3-L7-VL2-L8-CL; wherein VL1 is a first immunoglobulin light chain variable region that specifically binds to a SARS-CoV-2 protein; VL2 is a second immunoglobulin light chain variable region that specifically binds to a SARS-CoV-2 protein; VL3 is a third immunoglobulin light chain variable region that specifically binds to a SARS-CoV-2 protein; VH1 is a first immunoglobulin heavy chain variable region that specifically binds to a SARS-CoV-2 protein; VH2 is a second immunoglobulin heavy chain variable region that specifically binds to a SARS-CoV-2 protein; VH3 is a third immunoglobulin heavy chain variable region that specifically binds to a SARS-CoV-2 protein; Fc is a region comprising an immunoglobulin heavy chain constant region 2 (CH2), an immunoglobulin heavy chain constant region 3 (CH3), and optionally, an immunoglobulin hinge; CH1 is an immunoglobulin heavy chain constant region 1; CL is an immunoglobulin light chain constant region; and L1-L8 are amino acid linkers.

In some aspects, VH1 and VH3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345). In some aspects, VL1 and VL3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some aspects, VH1 and VH3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345), and VL1 and VL3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some aspects, VH2 comprises different CDR1, CDR2 and/or CDR3 sequences than VH1 and VH3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345), and/or VL2 comprises different CDR1, CDR2 and/or CDR3 sequences than VL1 and VL3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1 and VH2 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345). In some aspects, VL1 and VL2 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some aspects, VH1 and VH2 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345), and VL1 and VL2 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some aspects, VH3 comprises different CDR1, CDR2 and/or CDR3 sequences than VH1 and VH2 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345), and/or VL3 comprises different CDR1, CDR2 and/or CDR3 sequences than VL1 and VL2 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH2 and VH3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345). In some aspects, VL2 and VL3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some aspects, VH2 and VH3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345), and VL2 and VL3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some aspects, VH1 comprises different CDR1, CDR2 and/or CDR3 sequences than VH2 and VH3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345), and/or VL1 comprises different CDR1, CDR2 and/or CDR3 sequences than VL2 and VL3 (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and VH3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345). In some aspects, VL1, VL2 and VL3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some aspects, VH1, VH2 and VH3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345), and VL1, VL2 and VL3 each comprise the same CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and VH3 each comprise different CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345). In some aspects, VL1, VL2 and VL3 each comprise different CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some aspects, VH1, VH2 and VH3 each comprise different CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2-4, 10-12, 18-20, 26-28, 34-36, 148-150, 160-162, 168-170, 176-178, 184-186, 193-195, 201-203, 209-211, 217-219, 225-227, 233-235, 241-243, 249-251, 257-259, 265-267, 273-275, 281-283, 289-291, 297-299, 319-321, 327-329, 335-337 and 343-345), and VL1, VL2 and VL3 each comprise different CDR1, CDR2 and/or CDR3 sequences (e.g., an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs: 6-8, 14-16, 22-24, 30-32, 38-40, 152-154, 156-158, 164-166, 172-174, 180-182, 197-199, 205-207, 213-215, 221-223, 229-231, 237-239, 245-247, 253-255, 261-263, 269-271, 277-279, 285-287, 293-295, 301-303, 323-325, 331-333, 339-341 and 347-349). In some preferred aspects, CDR1, CDR2 and CDR3 of each of VH1, VH2, VH3, VL1, VL2, and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of the corresponding amino acid sequences specified above.

In some aspects, VH1, VH2 and/or VH3 (VH1; V12; VH3; VH1 and VH2; VH1 and VH3; VH2 and VH3; VH1, VH2 and VH3) each independently comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VL1, VL2 and/or VL3 (VL1; VL2; VL3; VL1 and VL2; VL1 and VL3; VL2 and VL3; VL1, VL2 and VL3) each independently comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) each independently comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345; and VL1, VL2 and/or VL3 (VL1; VL2; VL3; VL1 and VL2; VL1 and VL3; VL2 and VL3; VL1, VL2 and VL3) each independently comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and/or a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and VH3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20. In some preferred aspects, VH1, VH2 and VH3 each comprise a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20. As used herein, “at least 90% identity” includes at least 91%, 92%, 93%, 94%, 95%, 96%, 97%, 98%, 99% and 100% identity to the recited reference sequence. In some particularly preferred aspects, VH1, VH2 and VH3 each comprise a CDR1 comprising the amino acid sequence of SEQ ID NO:18; a CDR2 comprising the amino acid sequence of SEQ ID NO:19; and a CDR3 comprising the amino acid sequence of SEQ ID NO:20.

In some aspects, VL1, VL2 and VL3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24. In some preferred aspects, VL1, VL2 and VL3 each comprise a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24. As used herein, “at least 90% identity” includes at least 91%, 92%, 93%, 94%, 95%, 96%, 97%, 98%, 99% and 100% identity to the recited reference sequence. In some particularly preferred aspects, VL1, VL2 and VL3 each comprise a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24.

In some aspects, VH1, VH2 and VH3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NOs:20; and VL1, VL2 and VL3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24. In some preferred aspects, VH1, VH2 and VH3 each comprise a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20; and VL1, VL2 and VL3 each comprise a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24. As used herein, “at least 90% identity” includes at least 91%, 92%, 93%, 94%, 95%, 96%, 97%, 98%, 99% and 100% identity to the recited reference sequence. In some particularly preferred aspects, VH1, VH2 and VH3 each comprise a CDR1 comprising the amino acid sequence of SEQ ID NO:18; a CDR2 comprising the amino acid sequence of SEQ ID NO:19; and a CDR3 comprising the amino acid sequence of SEQ ID NO:20; and VL1, VL2 and VL3 each comprise a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24.

In some aspects, VH1 and VH3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; and VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28. In some preferred aspects, VH1 and VH3 each comprise a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20; and VH2 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28. As used herein, “at least 90% identity” includes at least 91%, 92%, 93%, 94%, 95%, 96%, 97%, 98%, 99% and 100% identity to the recited reference sequence. In some particularly preferred aspects, VH1 and VH3 each comprise a CDR1 comprising the amino acid sequence of SEQ ID NO:18; a CDR2 comprising the amino acid sequence of SEQ ID NO:19; and a CDR3 comprising the amino acid sequence of SEQ ID NO:20; and VH2 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28.

In some aspects, VL1 and VL3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32. In some preferred aspects, VL1 and VL3 each comprise a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL2 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32. As used herein, “at least 90% identity” includes at least 91%, 92%, 93%, 94%, 95%, 96%, 97%, 98%, 99% and 100% identity to the recited reference sequence. In some particularly preferred aspects, VL1 and VL3 each comprise a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL2 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32.

In some aspects, VH1 and VH3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; VL1 and VL3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32. In some preferred aspects, VH1 and VH3 each comprise a CDR1 comprising an amino acid sequence having at least 90% identity to SEQ ID NO: 18; a CDR2 comprising an amino acid sequence having at least 90% identity to SEQ ID NO: 19; and a CDR3 comprising an amino acid sequence having at least 90% identity to SEQ ID NO:20; and VH2 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VL1 and VL3 each comprise a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL2 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32. As used herein, “at least 90% identity” includes at least 91%, 92%, 93%, 94%, 95%, 96%, 97%, 98%, 99% and 100% identity to the recited reference sequence. In some particularly preferred aspects, VH1 and VH3 each comprise a CDR1 comprising the amino acid sequence of SEQ ID NO:18; a CDR2 comprising the amino acid sequence of SEQ ID NO:19; and a CDR3 comprising the amino acid sequence of SEQ ID NO:20; VH2 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VL1 and VL3 each comprise a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL2 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32.

In some aspects, VH1 and VH2 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; and VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28. In some preferred aspects, VH1 and VH2 each comprise a CDR1 comprising an amino acid sequence having at least 90% identity to SEQ ID NO:18; a CDR2 comprising an amino acid sequence having at least 90% identity to SEQ ID NO: 19; and a CDR3 comprising an amino acid sequence having at least 90% identity to SEQ ID NO:20; and VH3 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28. As used herein, “at least 90% identity” includes at least 91%, 92%, 93%, 94%, 95%, 96%, 97%, 98%, 99% and 100% identity to the recited reference sequence. In some particularly preferred aspects, VH1 and VH2 each comprise a CDR1 comprising the amino acid sequence of SEQ ID NO:18; a CDR2 comprising the amino acid sequence of SEQ ID NO:19; and a CDR3 comprising the amino acid sequence of SEQ ID NO:20; and VH3 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28.

In some aspects, VL1 and VL2 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32. In some preferred aspects, VL1 and VL2 each comprise a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL3 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32. As used herein, “at least 90% identity” includes at least 91%, 92%, 93%, 94%, 95%, 96%, 97%, 98%, 99% and 100% identity to the recited reference sequence. In some particularly preferred aspects, VL1 and VL2 each comprise a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL3 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32.

In some aspects, VH1 and VH2 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; VL1 and VL2 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32. In some preferred aspects, VH1 and VH2 each comprise a CDR1 comprising an amino acid sequence having at least 90% identity to SEQ ID NO:18; a CDR2 comprising an amino acid sequence having at least 90% identity to SEQ ID NO: 19; and a CDR3 comprising an amino acid sequence having at least 90% identity to SEQ ID NO:20; VH3 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VL1 and VL2 each comprise a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL3 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32. As used herein, “at least 90% identity” includes at least 91%, 92%, 93%, 94%, 95%, 96%, 97%, 98%, 99% and 100% identity to the recited reference sequence. In some particularly preferred aspects, VH1 and VH2 each comprise a CDR1 comprising the amino acid sequence of SEQ ID NO:18; a CDR2 comprising the amino acid sequence of SEQ ID NO:19; and a CDR3 comprising the amino acid sequence of SEQ ID NO:20; VH3 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VL1 and VL2 each comprise a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL3 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32.

In some aspects, VH2 and VH3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; and VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28. In some preferred aspects, VH2 and VH3 each comprise a CDR1 comprising an amino acid sequence having at least 90% identity to SEQ ID NO:18; a CDR2 comprising an amino acid sequence having at least 90% identity to SEQ ID NO: 19; and a CDR3 comprising an amino acid sequence having at least 90% identity to SEQ ID NO:20; and VH1 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28. As used herein, “at least 90% identity” includes at least 91%, 92%, 93%, 94%, 95%, 96%, 97%, 98%, 99% and 100% identity to the recited reference sequence. In some particularly preferred aspects, VH2 and VH3 each comprise a CDR1 comprising the amino acid sequence of SEQ ID NO:18; a CDR2 comprising the amino acid sequence of SEQ ID NO:19; and a CDR3 comprising the amino acid sequence of SEQ ID NO:20; and VH1 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28.

In some aspects, VL2 and VL3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32. In some preferred aspects, VL2 and VL3 each comprise a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL1 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32. As used herein, “at least 90% identity” includes at least 91%, 92%, 93%, 94%, 95%, 96%, 97%, 98%, 99% and 100% identity to the recited reference sequence. In some particularly preferred aspects, VL2 and VL3 each comprise a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL1 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32.

In some aspects, VH2 and VH3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; VL2 and VL3 each comprise a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32. In some preferred aspects, VH2 and VH3 each comprise a CDR1 comprising an amino acid sequence having at least 90% identity to SEQ ID NO:18; a CDR2 comprising an amino acid sequence having at least 90% identity to SEQ ID NO: 19; and a CDR3 comprising an amino acid sequence having at least 90% identity to SEQ ID NO:20; and VH1 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VL2 and VL3 each comprise a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL1 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32. As used herein, “at least 90% identity” includes at least 91%, 92%, 93%, 94%, 95%, 96%, 97%, 98%, 99% and 100% identity to the recited reference sequence. In some particularly preferred aspects, VH2 and VH3 each comprise a CDR1 comprising the amino acid sequence of SEQ ID NO:18; a CDR2 comprising the amino acid sequence of SEQ ID NO:19; and a CDR3 comprising the amino acid sequence of SEQ ID NO:20; VH1 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VL2 and VL3 each comprise a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL1 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:10; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:11; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12; VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; and VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28. In some preferred aspects, VH1 comprises a CDR1 comprising an amino acid sequence having at least 90% identity to SEQ ID NO:10; a CDR2 comprising an amino acid sequence having at least 90% identity to SEQ ID NO: 11; and a CDR3 comprising an amino acid sequence having at least 90% identity to SEQ ID NO:12; VH2 comprises a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20; and VH3 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28. As used herein, “at least 90% identity” includes at least 91%, 92%, 93%, 94%, 95%, 96%, 97%, 98%, 99% and 100% identity to the recited reference sequence. In some particularly preferred aspects, VH1 comprises a CDR1 comprising the amino acid sequence of SEQ ID NO:10; a CDR2 comprising the amino acid of SEQ ID NO:11; and a CDR3 comprising the amino acid sequence of SEQ ID NO:12; VH2 comprises a CDR1 comprising the amino acid sequence of SEQ ID NO:18; a CDR2 comprising the amino acid sequence of SEQ ID NO:19; and a CDR3 comprising the amino acid sequence of SEQ ID NO:20; and VH3 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28.

In some aspects, VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:14; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:15 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:16; VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32. In some preferred aspects, VL1 comprises a CDR1 comprising an amino acid sequence having at least 90% identity to SEQ ID NO:14; a CDR2 comprising an amino acid sequence having at least 90% identity to SEQ ID NO:15 (having the sequence of GTN); and a CDR3 comprising an amino acid sequence having at least 90% identity to SEQ ID NO:16; VL2 comprises a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL3 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32. As used herein, “at least 90% identity” includes at least 91%, 92%, 93%, 94%, 95%, 96%, 97%, 98%, 99% and 100% identity to the recited reference sequence. In some particularly preferred aspects, VL1 comprises a CDR1 comprising the amino acid sequence of SEQ ID NO:14; a CDR2 comprising the amino acid sequence of SEQ ID NO:15 (having the sequence of GTN); and a CDR3 comprising the amino acid sequence of SEQ ID NO:16; VL2 comprises a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL3 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:10; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:11; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12; VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:14; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:15 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:16; VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32. In some preferred aspects, VH1 comprises a CDR1 comprising an amino acid sequence having at least 90% identity to SEQ ID NO:10; a CDR2 comprising an amino acid sequence having at least 90% identity to SEQ ID NO: 11; and a CDR3 comprising an amino acid sequence having at least 90% identity to SEQ ID NO:12; VH2 comprises a CDR1 comprising an amino acid sequence having at least 90% identity to SEQ ID NO: 18; a CDR2 comprising an amino acid sequence having at least 90% identity to SEQ ID NO: 19; and a CDR3 comprising an amino acid sequence having at least 90% identity to SEQ ID NO:20; VH3 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VL1 comprises a CDR1 comprising an amino acid sequence having at least 90% identity to SEQ ID NO:14; a CDR2 comprising an amino acid sequence having at least 90% identity to SEQ ID NO:15 (having the sequence of GTN); and a CDR3 comprising an amino acid sequence having at least 90% identity to SEQ ID NO:16; VL2 comprises a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL3 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32. As used herein, “at least 90% identity” includes at least 91%, 92%, 93%, 94%, 95%, 96%, 97%, 98%, 99% and 100% identity to the recited reference sequence. In some particularly preferred aspects, VH1 comprises a CDR1 comprising the amino acid sequence of SEQ ID NO:10; a CDR2 comprising the amino acid of SEQ ID NO:11; and a CDR3 comprising the amino acid sequence of SEQ ID NO:12; VH2 comprises a CDR1 comprising the amino acid sequence of SEQ ID NO:18; a CDR2 comprising the amino acid sequence of SEQ ID NO:19; and a CDR3 comprising the amino acid sequence of SEQ ID NO:20; VH3 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VL1 comprises a CDR1 comprising the amino acid sequence of SEQ ID NO:14; a CDR2 comprising the amino acid sequence of SEQ ID NO:15 (having the sequence of GTN); and a CDR3 comprising the amino acid sequence of SEQ ID NO:16; VL2 comprises a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL3 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:10; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:11; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12; VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; and VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20. In some preferred aspects, VH1 comprises a CDR1 comprising an amino acid sequence having at least 90% identity to SEQ ID NO:10; a CDR2 comprising an amino acid sequence having at least 90% identity to SEQ ID NO: 11; and a CDR3 comprising an amino acid sequence having at least 90% identity to SEQ ID NO:12; VH2 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; and VH3 comprises a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20. As used herein, “at least 90% identity” includes at least 91%, 92%, 93%, 94%, 95%, 96%, 97%, 98%, 99% and 100% identity to the recited reference sequence. In some particularly preferred aspects, VH1 comprises a CDR1 comprising the amino acid sequence of SEQ ID NO:10; a CDR2 comprising the amino acid sequence of SEQ ID NO:11; and a CDR3 comprising the amino acid sequence of SEQ ID NO:12; VH2 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; and VH3 comprises a CDR1 comprising the amino acid sequence of SEQ ID NO:18; a CDR2 comprising the amino acid sequence of SEQ ID NO:19; and a CDR3 comprising the amino acid sequence of SEQ ID NO:20.

In some aspects, VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:14; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:15 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:16; VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24. In some preferred aspects, VL1 comprises a CDR1 comprising an amino acid sequence having at least 90% identity to SEQ ID NO:14; a CDR2 comprising an amino acid sequence having at least 90% identity to SEQ ID NO:15 (having the sequence of GTN); and a CDR3 comprising an amino acid sequence having at least 90% identity to SEQ ID NO:16; VL2 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32; and VL3 comprises a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24. As used herein, “at least 90% identity” includes at least 91%, 92%, 93%, 94%, 95%, 96%, 97%, 98%, 99% and 100% identity to the recited reference sequence. In some particularly preferred aspects, VL1 comprises a CDR1 comprising the amino acid sequence of SEQ ID NO:14; a CDR2 comprising the amino acid sequence of SEQ ID NO:15 (having the sequence of GTN); and a CDR3 comprising the amino acid sequence of SEQ ID NO:16; VL2 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32; and VL3 comprises a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:10; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:11; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12; VH2 comprises a CDR1 comprising an amino acid sequence having at least 80%, at least 85%, at least 90%, at least 91%, at least 92%, at least 93%, at least 94%, at least 95%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; and VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; VL1 comprises a CDR1 comprising an amino acid sequence having at least 80%, at least 85%, at least 91%, at least 92%, at least 93%, at least 94%, at least 90%, at least 95%, at least 96%, at least 97%, at least 98%, at least 99% or 100% identity to SEQ ID NO:14; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:15 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:16; VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24. In some preferred aspects, VH1 comprises a CDR1 comprising an amino acid sequence having at least 90% identity to SEQ ID NO:10; a CDR2 comprising an amino acid sequence having at least 90% identity to SEQ ID NO:11; and a CDR3 comprising an amino acid sequence having at least 90% identity to SEQ ID NO:12; VH2 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VH3 comprises a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20; VL1 comprises a CDR1 comprising an amino acid sequence having at least 90% identity to SEQ ID NO:14; a CDR2 comprising an amino acid sequence having at least 90% identity to SEQ ID NO:15 (having the sequence of GTN); and a CDR3 comprising an amino acid sequence having at least 90% identity to SEQ ID NO:16; VL2 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32; and VL3 comprises a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24. As used herein, “at least 90% identity” includes at least 91%, 92%, 93%, 94%, 95%, 96%, 97%, 98%, 99% and 100% identity to the recited reference sequence. In some particularly preferred aspects, VH1 comprises a CDR1 comprising the amino acid sequence of SEQ ID NO:10; a CDR2 comprising the amino acid sequence of SEQ ID NO:11; and a CDR3 comprising the amino acid sequence of SEQ ID NO:12; VH2 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VH3 comprises a CDR1 comprising the amino acid sequence of SEQ ID NO:18; a CDR2 comprising the amino acid sequence of SEQ ID NO:19; and a CDR3 comprising the amino acid sequence of SEQ ID NO:20; VL1 comprises a CDR1 comprising the amino acid sequence of SEQ ID NO:14; a CDR2 comprising the amino acid sequence of SEQ ID NO:15 (having the sequence of GTN); and a CDR3 comprising the amino acid sequence of SEQ ID NO:16; VL2 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32; and VL3 comprises a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:10; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:11; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12; and VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20. In some preferred aspects, VH1 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VH2 comprises a CDR1 comprising an amino acid sequence having at least 90% identity to SEQ ID NO:10; a CDR2 comprising an amino acid sequence having at least 90% identity to SEQ ID NO:11; and a CDR3 comprising an amino acid sequence having at least 90% identity to SEQ ID NO:12; and VH3 comprises a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20. As used herein, “at least 90% identity” includes at least 91%, 92%, 93%, 94%, 95%, 96%, 97%, 98%, 99% and 100% identity to the recited reference sequence. In some particularly preferred aspects, VH1 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VH2 comprises a CDR1 comprising the amino acid sequence of SEQ ID NO:10; a CDR2 comprising the amino acid sequence of SEQ ID NO:11; and a CDR3 comprising the amino acid sequence of SEQ ID NO:12; and VH3 comprises a CDR1 comprising the amino acid sequence of SEQ ID NO:18; a CDR2 comprising the amino acid sequence of SEQ ID NO:19; and a CDR3 comprising the amino acid sequence of SEQ ID NO:20.

In some aspects, VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32; VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:14; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:15 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:16; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24. In some preferred aspects, VL1 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32; VL2 comprises a CDR1 comprising an amino acid sequence having at least 90% identity to SEQ ID NO: 14; a CDR2 comprising an amino acid sequence having at least 90% identity to SEQ ID NO: 15 (having the sequence of GTN); and a CDR3 comprising an amino acid sequence having at least 90% identity to SEQ ID NO:16; and VL3 comprises a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24. As used herein, “at least 90% identity” includes at least 91%, 92%, 93%, 94%, 95%, 96%, 97%, 98%, 99% and 100% identity to the recited reference sequence. In some particularly preferred aspects, VL1 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32; VL2 comprises a CDR1 comprising the amino acid sequence of SEQ ID NO:14; a CDR2 comprising the amino acid sequence of SEQ ID NO:15 (having the sequence of GTN); and a CDR3 comprising the amino acid sequence of SEQ ID NO:16; and VL3 comprises a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:10; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:11; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12; VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32; VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:14; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:15 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:16; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24. In some preferred aspects, VH1 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VH2 comprises a CDR1 comprising an amino acid sequence having at least 90% identity to SEQ ID NO: 10; a CDR2 comprising an amino acid sequence having at least 90% identity to SEQ ID NO: 11; and a CDR3 comprising an amino acid sequence having at least 90% identity to SEQ ID NO:12; VH3 comprises a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20; VL1 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32; VL2 comprises a CDR1 comprising an amino acid sequence having at least 90% identity to SEQ ID NO:14; a CDR2 comprising an amino acid sequence having at least 90% identity to SEQ ID NO:15 (having the sequence of GTN); and a CDR3 comprising an amino acid sequence having at least 90% identity to SEQ ID NO:16; and VL3 comprises a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24. As used herein, “at least 90% identity” includes at least 91%, 92%, 93%, 94%, 95%, 96%, 97%, 98%, 99% and 100% identity to the recited reference sequence. In some particularly preferred aspects, VH1 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VH2 comprises a CDR1 comprising the amino acid sequence of SEQ ID NO:10; a CDR2 comprising the amino acid sequence of SEQ ID NO:11; and a CDR3 comprising the amino acid sequence of SEQ ID NO:12; VH3 comprises a CDR1 comprising the amino acid sequence of SEQ ID NO:18; a CDR2 comprising the amino acid sequence of SEQ ID NO:19; and a CDR3 comprising the amino acid sequence of SEQ ID NO:20; VL1 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32; VL2 comprises a CDR1 comprising the amino acid sequence of SEQ ID NO:14; a CDR2 comprising the amino acid sequence of SEQ ID NO:15 (having the sequence of GTN); and a CDR3 comprising the amino acid sequence of SEQ ID NO:16; and VL3 comprises a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; and VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:10; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:11; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12. In some preferred aspects, VH1 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VH2 comprises a CDR1 comprising an amino acid sequence having at least 90% identity to SEQ ID NO:18; a CDR2 comprising an amino acid sequence having at least 90% identity to SEQ ID NO:19; and a CDR3 comprising an amino acid sequence having at least 90% identity to SEQ ID NO:20; and VH3 comprises a CDR1 comprising an amino acid sequence having at least 90% identity to SEQ ID NO:10; a CDR2 comprising an amino acid sequence having at least 90% identity to SEQ ID NO:11; and a CDR3 comprising an amino acid sequence having at least 90% identity to SEQ ID NO: 12. As used herein, “at least 90% identity” includes at least 91%, 92%, 93%, 94%, 95%, 96%, 97%, 98%, 99% and 100% identity to the recited reference sequence. In some particularly preferred aspects, VH1 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VH2 comprises a CDR1 comprising the amino acid sequence of SEQ ID NO:18; a CDR2 comprising the amino acid sequence of SEQ ID NO:19; and a CDR3 comprising the amino acid sequence of SEQ ID NO:20; and VH3 comprises a CDR1 comprising the amino acid sequence of SEQ ID NO:10; a CDR2 comprising the amino acid sequence of SEQ ID NO:11; and a CDR3 comprising the amino acid sequence of SEQ ID NO:12.

In some aspects, VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32; VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:14; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:15 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:16. In some preferred aspects, VL1 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32; VL2 comprises a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL3 comprises a CDR1 comprising an amino acid sequence having at least 90% identity to SEQ ID NO:14; a CDR2 comprising an amino acid sequence having at least 90% identity to SEQ ID NO:15 (having the sequence of GTN); and a CDR3 comprising an amino acid sequence having at least 90% identity to SEQ ID NO:16. As used herein, “at least 90% identity” includes at least 91%, 92%, 93%, 94%, 95%, 96%, 97%, 98%, 99% and 100% identity to the recited reference sequence. In some particularly preferred aspects, VL1 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32; VL2 comprises a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL3 comprises a CDR1 comprising the amino acid sequence of SEQ ID NO:14; a CDR2 comprising the amino acid sequence of SEQ ID NO:15 (having the sequence of GTN); and a CDR3 comprising the amino acid sequence of SEQ ID NO:16.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:26; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:27; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:28; VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:18; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:19; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:20; VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:10; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:11; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:12; VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:30; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:31 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:32; VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:22; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:23 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:24; and VL3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:14; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:15 (having the sequence of GTN); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:16. In some preferred aspects, VH1 comprises a CDR1 comprising an amino acid sequence having at least 90% identity to SEQ ID NO:26; a CDR2 comprising an amino acid sequence having at least 90% identity to SEQ ID NO:27; and a CDR3 comprising an amino acid sequence having at least 90% identity to SEQ ID NO:28; VH2 comprises a CDR1 comprising an amino acid sequence having at least 90% identity to SEQ ID NO: 18; a CDR2 comprising an amino acid sequence having at least 90% identity to SEQ ID NO:19; and a CDR3 comprising an amino acid sequence having at least 90% identity to SEQ ID NO:20; VH3 comprises a CDR1 comprising an amino acid sequence having at least 90% identity to SEQ ID NO:10; a CDR2 comprising an amino acid sequence having at least 90% identity to SEQ ID NO:11; and a CDR3 comprising an amino acid sequence having at least 90% identity to SEQ ID NO:12; VL1 comprises a CDR1 comprising an amino acid sequence having at least 90% identity to SEQ ID NO:30; a CDR2 comprising an amino acid sequence having at least 90% identity to SEQ ID NO:31 (having the sequence of DAS); and a CDR3 comprising an amino acid sequence having at least 90% identity to SEQ ID NO:32; VL2 comprises a CDR1 comprising an amino acid sequence having at least 90% identity to SEQ ID NO:22; a CDR2 comprising an amino acid sequence having at least 90% identity to SEQ ID NO:23 (having the sequence of GAS); and a CDR3 comprising an amino acid sequence having at least 90% identity to SEQ ID NO:24; and VL3 comprises a CDR1 comprising an amino acid sequence having at least 90% identity to SEQ ID NO:14; a CDR2 comprising an amino acid sequence having at least 90% identity to SEQ ID NO:15 (having the sequence of GTN); and a CDR3 comprising an amino acid sequence having at least 90% identity to SEQ ID NO:16. As used herein, “at least 90% identity” includes at least 91%, 92%, 93%, 94%, 95%, 96%, 97%, 98%, 99% and 100% identity to the recited reference sequence. In some particularly preferred aspects, VH1 comprises a CDR1 comprising the amino acid sequence of SEQ ID NO:26; a CDR2 comprising the amino acid sequence of SEQ ID NO:27; and a CDR3 comprising the amino acid sequence of SEQ ID NO:28; VH2 comprises a CDR1 comprising the amino acid sequence of SEQ ID NO:18; a CDR2 comprising the amino acid sequence of SEQ ID NO:19; and a CDR3 comprising the amino acid sequence of SEQ ID NO:20; VH3 comprises a CDR1 comprising the amino acid sequence of SEQ ID NO:10; a CDR2 comprising the amino acid sequence of SEQ ID NO:11; and a CDR3 comprising the amino acid sequence of SEQ ID NO:12; VL1 comprises a CDR1 comprising the amino acid sequence of SEQ ID NO:30; a CDR2 comprising the amino acid sequence of SEQ ID NO:31 (having the sequence of DAS); and a CDR3 comprising the amino acid sequence of SEQ ID NO:32; VL2 comprises a CDR1 comprising the amino acid sequence of SEQ ID NO:22; a CDR2 comprising the amino acid sequence of SEQ ID NO:23 (having the sequence of GAS); and a CDR3 comprising the amino acid sequence of SEQ ID NO:24; and VL3 comprises a CDR1 comprising the amino acid sequence of SEQ ID NO:14; a CDR2 comprising the amino acid sequence of SEQ ID NO:15 (having the sequence of GTN); and a CDR3 comprising the amino acid sequence of SEQ ID NO:16.

In some aspects, VH1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:148. In some aspects, VH1 comprises a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:149. In some aspects, VH1 comprises a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:150. In some aspects, VH2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:148. In some aspects, VH2 comprises a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:149. In some aspects, VH2 comprises a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:150. In some aspects, VH3 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:148. In some aspects, VH3 comprises a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:149. In some aspects, VH3 comprises a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:150. In some preferred aspects, CDR1, CDR2 and CDR3 of each of VH1, VH2, and VH3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects CDR1, CDR2 and CDR3 of each of VH1, VH2, and VH3 comprise or consist of a corresponding amino acid sequence specified above.

In some aspects, VL1 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:152. In some aspects, VL1 comprises a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:153. In some aspects, VL1 comprises a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:154. In some aspects, VL2 comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:152. In some aspects, VL2 comprises a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:153. In some aspects, VL2 comprises a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:154. In some aspects, VL3 comprises a CDR1 comprising an amino acid sequence having at least 80%, at least 85%, at least 90%, at least 91%, at least 92%, at least 93%, at least 94%, at least 95%, at least 99% or 100% identity to SEQ ID NO:152. In some aspects, VL3 comprises a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:153. In some aspects, VL3 comprises a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:154. In some preferred aspects, CDR1, CDR2 and CDR3 of each of VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:193; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:194; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:195; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:197; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:198 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:199. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:201; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:202; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:203; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:205; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:206 (having the sequence of DVS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:207. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:209; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:210; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:211; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:213; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:214 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:215. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:217; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:218; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:219; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:221; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:222 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:223. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:225; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:226; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:227; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:229; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:230 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:231. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:233; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:234; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:235; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:237; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:238 (having the sequence of AAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:239. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:241; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:242; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:243; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:245; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:246 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:247. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:249; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:250; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:251; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:253; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:254 (having the sequence of DDS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:255. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:257; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:258; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:259; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:261; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:262 (having the sequence of KDS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:263. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:265; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:266; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:267; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:269; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:270 (having the sequence of SAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:271. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:273; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:274; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:275; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:277; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:278 (having the sequence of SAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:279. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:281; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:282; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:283; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:285; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:286 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:287. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:289; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:290; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:291; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:293; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:294 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:295. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:297; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:298; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:299; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:301; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:302 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:303. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:319; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:320; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:321; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:323; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:324 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:325. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:327; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:328; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:329; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:331; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:332 (having the sequence of DAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:333. In some preferred aspects, CDR1, CDR2 and CDR3 of each of VH1, VH2, VH3, VL1, VL2, and VL3 have at least 90% identity with a corresponding amino acid sequence specified above. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:335; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:336; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:337; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:339; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:340 (having the sequence of SAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:341. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:343; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:344; and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:345; and VL1, VL2 and/or VL3 (VL1; VL2; VL3; VL1 and VL2; VL1 and VL3; VL2 and VL3; VL1, VL2 and VL3) comprises a CDR1 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:347; a CDR2 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:348 (having the sequence of GAS); and a CDR3 comprising an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:349. In some preferred aspects, CDR1, CDR2 and CDR3 of each of VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of VH1, VH2, VH3, VL1, VL2, and VL3 comprise or consist of a corresponding amino acid sequence specified above.

In some aspects, VH1 comprises the same heavy chain variable region as VH3. In some aspects, VL1 comprises the same light chain variable region as VL3. In some aspects, VH1 comprises the same heavy chain variable region as VH3, and VL1 comprises the same light chain variable region as VL3.

In some aspects, VH1 comprises the same heavy chain variable region as VH2. In some aspects, VL1 comprises the same light chain variable region as VL2. In some aspects, VH1 comprises the same heavy chain variable region as VH2, and VL1 comprises the same light chain variable region as VL2.

In some aspects, VH2 comprises the same heavy chain variable region as VH3. In some aspects, VL2 comprises the same light chain variable region as VL3. In some aspects, VH2 comprises the same heavy chain variable region as VH3, and VL2 comprises the same light chain variable region as VL3.

In some aspects, VH1, VH2 and VH3 each comprise the same heavy chain variable region. In some aspects, VL1, VL2 and VL3 each comprise the same light chain variable region. In some aspects, VH1, VH2 and VH3 each comprise the same heavy chain variable region, and VL1, VL2 and VL3 each comprise the same light chain variable region.

In some aspects, VH1, VH2 and VH3 each comprise different heavy chain variable regions. In some aspects, VL1, VL2 and VL3 each comprise different light chain variable regions. In some aspects, VH1, VH2 and VH3 each comprise different heavy chain variable regions, and VL1, VL2 and VL3 each comprise different light chain variable regions. In some aspects, VH1, VH2 and/or VH3 (VH1; V12; VH3; VH1 and V12; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359. In some preferred aspects, VH1, VH2, and VH3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, and VH3 comprise or consist of a corresponding amino acid sequence specified above. In some aspects, VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346. In some preferred aspects, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VL1, VL2, and VL3 comprise or consist of a corresponding amino acid sequence specified above. In some aspects, VH1, VH2 and/or VH3 (VH1; V12; VH3; VH1 and V12; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346. In some aspects, the sequence has a mutation at N62, for example, N62Q or N62Q at, for example, SEQ ID NO:175. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:17; and VL1, VL2 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:21. In some preferred aspects, VH1, VH2 and VH3 each comprise an amino acid sequence having at least 90% identity to SEQ ID NO:17; and VL1, VL2 and VL3 each comprise an amino acid sequence having at least 90% identity to SEQ ID NO:21. As used herein, “at least 90% identity” includes at least 91%, 92%, 93%, 94%, 95%, 96%, 97%, 98%, 99% and 100% identity to the recited reference sequence. In some particularly preferred aspects, VH1, VH2 and VH3 each comprise the amino acid sequence of SEQ ID NO:17; and VL1, VL2 and VL3 each comprise the amino acid sequence of SEQ ID NO:21.

In some aspects, VH1 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:17; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:25; VL1 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:21; and VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:29. In some preferred aspects, VH1 and VH3 each comprise an amino acid sequence having at least 90% identity to SEQ ID NO:17; VH2 comprises an amino acid sequence having at least 90% identity to SEQ ID NO:25; VL1 and VL3 each comprise an amino acid sequence having at least 90% identity to SEQ ID NO:21; and VL2 comprises an amino acid sequence having at least 90% identity to SEQ ID NO:29. As used herein, “at least 90% identity” includes at least 91%, 92%, 93%, 94%, 95%, 96%, 97%, 98%, 99% and 100% identity to the recited reference sequence. In some particularly preferred aspects, VH1 and VH3 each comprise the amino acid sequence of SEQ ID NO:17; VH2 comprises the amino acid sequence of SEQ ID NO:25; VL1 and VL3 each comprise the amino acid sequence of SEQ ID NO:21; and VL2 comprises the amino acid sequence of SEQ ID NO:29.

In some aspects, VH1 and VH2 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:17; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:25; VL1 and VL2 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:21; and VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:29. In some preferred aspects, VH1 and VH2 each comprise an amino acid sequence having at least 90% identity to SEQ ID NO:17; VH3 comprises an amino acid sequence having at least 90% identity to SEQ ID NO:25; VL1 and VL2 each comprise an amino acid sequence having at least 90% identity to SEQ ID NO:21; and VL3 comprises an amino acid sequence having at least 90% identity to SEQ ID NO:29. As used herein, “at least 90% identity” includes at least 91%, 92%, 93%, 94%, 95%, 96%, 97%, 98%, 99% and 100% identity to the recited reference sequence. In some particularly preferred aspects, VH1 and VH2 each comprise the amino acid sequence of SEQ ID NO:17; VH3 comprises the amino acid sequence of SEQ ID NO:25; VL1 and VL2 each comprise the amino acid sequence of SEQ ID NO:21; and VL3 comprises the amino acid sequence of SEQ ID NO:29.

In some aspects, VH2 and VH3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:17; VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:25; VL2 and VL3 each comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:21; and VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:29. In some preferred aspects, VH2 and VH3 each comprise an amino acid sequence having at least 90% identity to SEQ ID NO:17; VH1 comprises an amino acid sequence having at least 90% identity to SEQ ID NO:25; VL2 and VL3 each comprise an amino acid sequence having at least 90% identity to SEQ ID NO:21; and VL1 comprises an amino acid sequence having at least 90% identity to SEQ ID NO:29. As used herein, “at least 90% identity” includes at least 91%, 92%, 93%, 94%, 95%, 96%, 97%, 98%, 99% and 100% identity to the recited reference sequence. In some particularly preferred aspects, VH2 and VH3 each comprise the amino acid sequence of SEQ ID NO:17; VH1 comprises the amino acid sequence of SEQ ID NO:25; VL2 and VL3 each comprise the amino acid sequence of SEQ ID NO:21; and VL1 comprises the amino acid sequence of SEQ ID NO:29.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:9; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:17; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:25; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:13; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:21; and VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:29. In some preferred aspects, VH1 comprises an amino acid sequence having at least 90% identity to SEQ ID NO:9; VH2 comprises an amino acid sequence having at least 90% identity to SEQ ID NO: 17; VH3 comprises an amino acid sequence having at least 90% identity to SEQ ID NO:25; VL1 comprises an amino acid sequence having at least 90% identity to SEQ ID NO:13; VL2 comprises an amino acid sequence having at least 90% identity to SEQ ID NO:21; and VL3 comprises an amino acid sequence having at least 90% identity to SEQ ID NO:29. As used herein, “at least 90% identity” includes at least 91%, 92%, 93%, 94%, 95%, 96%, 97%, 98%, 99% and 100% identity to the recited reference sequence. In some particularly preferred aspects, VH1 comprises the amino acid sequence of SEQ ID NO:9; VH2 comprises the amino acid sequence of SEQ ID NO:17; VH3 comprises the amino acid sequence of SEQ ID NO:25; VL1 comprises the amino acid sequence of SEQ ID NO:13; VL2 comprises the amino acid sequence of SEQ ID NO:21; and VL3 comprises the amino acid sequence of SEQ ID NO:29.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:9; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:25; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:17; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:13; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:29; and VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:21. In some preferred aspects, VH1 comprises an amino acid sequence having at least 90% identity to SEQ ID NO:9; VH2 comprises an amino acid sequence having at least 90% identity to SEQ ID NO:25; VH3 comprises an amino acid sequence having at least 90% identity to SEQ ID NO: 17; VL1 comprises an amino acid sequence having at least 90% identity to SEQ ID NO: 13; VL2 comprises an amino acid sequence having at least 90% identity to SEQ ID NO:29; and VL3 comprises an amino acid sequence having at least 90% identity to SEQ ID NO:21. As used herein, “at least 90% identity” includes at least 91%, 92%, 93%, 94%, 95%, 96%, 97%, 98%, 99% and 100% identity to the recited reference sequence. In some particularly preferred aspects, VH1 comprises the amino acid sequence of SEQ ID NO:9; VH2 comprises the amino acid sequence of SEQ ID NO:25; VH3 comprises the amino acid sequence of SEQ ID NO:17; VL1 comprises the amino acid sequence of SEQ ID NO:13; VL2 comprises the amino acid sequence of SEQ ID NO:29; and VL3 comprises the amino acid sequence of SEQ ID NO:21.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:25; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:9; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:17; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:29; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:13; and VL3 comprises an amino acid sequence having at least 80%, at least 85%, at least 90%, at least 95%, at least 99% or 100% identity to SEQ ID NO:21. In some preferred aspects, VH1 comprises an amino acid sequence having at least 90% identity to SEQ ID NO:25; VH2 comprises an amino acid sequence having at least 90% identity to SEQ ID NO:9; VH3 comprises an amino acid sequence having at least 90% identity to SEQ ID NO: 17; VL1 comprises an amino acid sequence having at least 90% identity to SEQ ID NO:29; VL2 comprises an amino acid sequence having at least 90% identity to SEQ ID NO:13; and VL3 comprises an amino acid sequence having at least 90% identity to SEQ ID NO:21. As used herein, “at least 90% identity” includes at least 91%, 92%, 93%, 94%, 95%, 96%, 97%, 98%, 99% and 100% identity to the recited reference sequence. In some particularly preferred aspects, VH1 comprises the amino acid sequence of SEQ ID NO:25; VH2 comprises the amino acid sequence of SEQ ID NO:9; VH3 comprises the amino acid sequence of SEQ ID NO:17; VL1 comprises the amino acid sequence of SEQ ID NO:29; VL2 comprises the amino acid sequence of SEQ ID NO:13; and VL3 comprises the amino acid sequence of SEQ ID NO:21.

In some aspects, VH1 comprises an amino acid sequence having at least 80%, at least 85%, at least 90%, at least 95%, at least 99% or 100% identity to SEQ ID NO:25; VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:17; VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:9; VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:29; VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:21; and VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:13. In some preferred aspects, VH1 comprises an amino acid sequence having at least 90% identity to SEQ ID NO:25; VH2 comprises an amino acid sequence having at least 90 identity to SEQ ID NO: 17; VH3 comprises an amino acid sequence having at least 90% identity to SEQ ID NO:9; VL1 comprises an amino acid sequence having at least 90% identity to SEQ ID NO:29; VL2 comprises an amino acid sequence having at least 90% identity to SEQ ID NO:21; and VL3 comprises an amino acid sequence having at least 90% identity to SEQ ID NO: 13. As used herein, “at least 90% identity” includes at least 91%, 92%, 93%, 94%, 95%, 96%, 97%, 98%, 99% and 100% identity to the recited reference sequence. In some particularly preferred aspects, VH1 comprises the amino acid sequence of SEQ ID NO:25; VH2 comprises the amino acid sequence of SEQ ID NO:17; VH3 comprises the amino acid sequence of SEQ ID NO:9; VL1 comprises the amino acid sequence of SEQ ID NO:29; VL2 comprises the amino acid sequence of SEQ ID NO:21; and VL3 comprises the amino acid sequence of SEQ ID NO:13.

In some aspects, VH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:147. In some aspects, VH2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:147. In some aspects, VH3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:147. In some aspects, VL1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:151. In some aspects, VL2 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:151. In some aspects, VL3 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:151. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:192; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:196. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:200; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:204. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:208; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:212. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:216; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:220. In some preferred aspects, VH1, VH2, VH3, VL1, VL2, and VL3 have at least 90% identity with the corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:224; and VL1, VL2 and/or VL3 comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:228. In some preferred aspects, VH1, VH2, VH3, VL1, VL2, and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of the corresponding amino acid sequences specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:232; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:236. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:240; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:244. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:248; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:252. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:256; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:260. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:264; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:268. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:272; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:276. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:280; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:284. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:288; and VL1, VL2 and/or VL3 comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:292. In some preferred aspects, VH1, VH2, VH3, VL1, VL2, and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of the corresponding amino acid sequences specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:296; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:300. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:304; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:305. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:306; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:307. In some preferred aspects, VH1, VH2, VH3, VL1, VL2, and VL3 have at least 90% identity with a corresponding amino acid sequence specified above. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:308; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:309. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:310; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:311. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:312; and VL1, VL2 and/or VL3 comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:313. In some preferred aspects, VH1, VH2, VH3, VL1, VL2, and VL3 have at least 90% identity with the corresponding amino acid sequences specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of the corresponding amino acid sequences specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:314; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:315. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:316; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:317. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:318; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:322. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:326; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:330. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; V12; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:334; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:338. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, VH1, VH2 and/or VH3 (VH1; VH2; VH3; VH1 and VH2; VH1 and VH3; VH2 and VH3; VH1, VH2 and VH3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:342; and VL1, VL2 and/or VL3 (VL1; VL2; VL3; VL1 and VL2; VL1 and VL3; VL2 and VL3; VL1, VL2 and VL3) comprise an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:346. In some preferred aspects, VH1, VH2, VH3, VL1, VL2, and VL3 have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, VH1, VH2, VH3, VL1, VL2, and VL3 comprise or consist of a corresponding amino acid sequence specified above.

In some aspects, an antigen binding polypeptide or antigen binding polypeptide complex provided herein comprises one or more amino acid sequences having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:81-124. In some preferred aspects, the antigen binding polypeptide or antigen binding polypeptide complex has at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the antigen binding polypeptide or antigen binding polypeptide complex comprises or consists of a corresponding amino acid sequence specified above.

In some aspects, an antigen binding polypeptide or antigen binding polypeptide complex provided herein is encoded by one or more polynucleotides having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:125-146. In some preferred aspects, the antigen binding polypeptide or antigen binding polypeptide complex is encoded by a polynucleotide that has at least 90% identity with a corresponding sequence specified above. As used herein, “at least 90% identity” includes at least 91%, 92%, 93%, 94%, 95%, 96%, 97%, 98%, 99% and 100% identity to the recited reference sequence. In some particularly preferred aspects, the antigen binding polypeptide or antigen binding polypeptide complex is encoded by a polynucleotide that comprises or consists of a corresponding sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:121 or 122; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:119 or 120; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:115 or 116; and/or the fourth polypeptide comprises an amino acid sequence having at least 80%, at least 85%, at least 90%, at least 95%, at least 99% or 100% identity to SEQ ID NO:117 or 118. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:121 or 122; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:119 or 120; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:123 or 124; and/or the fourth polypeptide comprises an amino acid sequence having at least 80%, at least 85%, at least 90%, at least 95%, at least 99% or 100% identity to SEQ ID NO:113 or 114. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:97 or 98; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:99 or 100; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:113 or 114; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:123 or 124. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:121 or 122; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:119 or 120; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:93 or 94; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:95 or 96. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:97 or 98; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:99 or 100; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:115 or 116; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:117 or 118. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:97 or 98; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:99 or 100; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:119 or 121; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:121 or 122. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:97 or 98; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:99 or 100; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:113 or 114; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:95 or 96 or 122. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:89 or 90; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:87 or 88; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:113 or 114; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:95 or 96. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:89 or 90; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:87 or 88; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:119 or 120; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO: 121 or 122. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:89 or 90; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:87 or 88; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:93 or 94; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:113 or 114. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:99 or 100; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:97 or 98; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:109 or 110; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:111 or 112. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects, the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:99 or 100; the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:97 or 98; the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:83 or 84; and/or the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:85 or 86. In some preferred aspects, the first, second, third and fourth polypeptides have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the first, second, third and fourth polypeptides comprise or consist of a corresponding amino acid sequence specified above.

In some aspects of any of the antigen binding polypeptide or antigen binding polypeptide complexes provided herein, CL comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43. In some preferred aspects, the CL has at least 90% identity to SEQ ID NO:43. In some particularly preferred aspects, the CL comprises or consists of SEQ ID NO: 43.

In some aspects of any of the antigen binding polypeptide or antigen binding polypeptide complexes provided herein, CH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42. In some preferred aspects, the CH1 has at least 90% identity to SEQ ID NO:42. In some particularly preferred aspects, the CH1 comprises or consists of SEQ ID NO: 42.

In some aspects of any of the antigen binding polypeptide or antigen binding polypeptide complexes provided herein, Fc comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44, 45 or 187. In some preferred aspects, the Fc has at least 90% identity to SEQ ID NO:44, 45 or 187. As used herein, “at least 90% identity” includes at least 91%, 92%, 93%, 94%, 95%, 96%, 97%, 98%, 99% and 100% identity to the recited reference sequence. In some particularly preferred aspects, the Fc comprises or consists of SEQ ID NO:44, 45 or 187.

In some aspects of any of the antigen binding polypeptide or antigen binding polypeptide complexes provided herein, CL comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; and CH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42. In some preferred aspects, the CL has at least 90% identity to SEQ ID NO:43 and the CH1 has at least 90% identity to SEQ ID NO:42. As used herein, “at least 90% identity” includes at least 91%, 92%, 93%, 94%, 95%, 96%, 97%, 98%, 99% and 100% identity to the recited reference sequence. In some particularly preferred aspects, the CL comprises or consists of SEQ ID NO: 43 and the CH1 comprises or consists of SEQ ID NO: 42.

In some aspects of any of the antigen binding polypeptide or antigen binding polypeptide complexes provided herein, CL comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:43; CH1 comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:42; and Fc comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:44, 45 or 187. In some preferred aspects, the CL has at least 90% identity to SEQ ID NO:43, the CH1 has at least 90% identity to SEQ ID NO:42 and the Fc has at least 90% identity to SEQ ID NO:44, 45 or 187. As used herein, “at least 90% identity” includes at least 91%, 92%, 93%, 94%, 95%, 96%, 97%, 98%, 99% and 100% identity to the recited reference sequence. In some particularly preferred aspects, the CL comprises or consists of SEQ ID NO: 43, the CH1 comprises or consists of SEQ ID NO: 42, and the Fc comprises or consists of SEQ ID NO:44, 45 or 187.

Molecular biology and recombinant DNA methods for making, screening and engineering an antigen binding polypeptide or antigen binding polypeptide complex (e.g., an antibody or antigen binding fragment thereof) containing such sequences are well known and described, for example, in Adair et al. Human Antibodies, 5(1-2):41-47, 1994; Kostelny et al., J Immunol, 148(5):1547-1553, 1992, Shiraiwa et al., Methods, 154:10-20, 2019; and Zola, “Monoclonal Antibodies: A Manual of Techniques,” 1987, 1st Ed., CRC Press; and Steinitz, Human Antibodies, 18(1-2):1-10, 2009.

As used herein, an antigen binding polypeptide or antigen binding polypeptide complex (e.g., an antibody or antigen binding fragment thereof), or region or domain thereof that “specifically binds” refers to its association with an epitope by its antigen binding domain, and that the binding entails some complementarity between the antigen binding domain and the epitope. Specific binding to an epitope occurs where there is binding to that epitope via its antigen binding domain more readily than there would be binding to a random, unrelated epitope.

As used herein, an “epitope” refers to a localized region of an antigen to which an antigen binding polypeptide or antigen binding polypeptide complex (e.g., antibody or antigen binding fragment thereof) can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). See, e.g., Giege R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303; Meth Enzymol (1985) volumes 114 & 115, eds Wyckoff H W et al., U.S. Pub. No. 2004/0014194), Bricogne G (1993) Acta Crystallogr D Biol Crystallogr 49(Pt 1): 37-60, Bricogne G (1997) Meth Enzymol 276A: 361-423, ed Carter C W, and Roversi et al., (2000) Acta Crystallogr D Biol Crystallogr 56(Pt 10): 1316-1323 (X-ray diffraction crystallography studies); and Champe et al., (1995) J Biol Chem 270: 1388-1394 and Cunningham B C & Wells J A (1989) Science 244: 1081-1085 (mutagenesis mapping).

Specific binding can be represented by a “binding affinity.” Binding affinity refers to an intrinsic binding affinity which reflects a 1:1 interaction between members of a binding pair (e.g., an antigen binding polypeptide complex and an antigen). Binding affinity can be measured and/or expressed in several ways known in the art, including, but not limited to, equilibrium dissociation constant (K_{D}). K_{D} is calculated from the quotient of k_{off}/kₒₙ, where kₒₙ refers to the association rate constant of, e.g., an antigen binding polypeptide complex to an antigen, and k_{off} refers to the dissociation of, e.g., an antigen binding polypeptide complex from an antigen. The kₒₙ and k_{off} can be determined by techniques known to one of ordinary skill in the art, such as Octet® BLI, BIAcore® or KinExA.

Accordingly, in some aspects, an antigen binding polypeptide complex provided herein is an antibody or antigen binding fragment thereof. In some aspects, an antigen binding polypeptide provided herein is part of an antibody or antigen binding fragment thereof.

In some aspects, the antibody or antigen binding fragment thereof specifically binds to an antigen with an equilibrium dissociation constant (K_{D}) of from about 10 μM to about 1 μM. In some aspects, the antibody is IgG, IgM, IgE, IgA or IgD. In some aspects, the IgG is IgG1, IgG2, IgG3 or IgG4. In some aspects, the antigen binding fragment is a Fab, scFab, Fab′, F(ab′)₂, Fv or scFv. In some aspects, the antibody is human or humanized.

In some aspects, an antigen binding polypeptide complex provided herein (e.g., an antibody or antigen binding fragment thereof) is trivalent.

In some aspects, an antigen binding polypeptide complex provided herein (e.g., an antibody or antigen binding fragment thereof) is monospecific, bispecific or trispecific.

In some aspects, an antigen binding polypeptide or antigen binding polypeptide complex provided herein potently inhibits one or more SARS-CoV-2 virus variants.

Also provided herein is an anti-SARS-CoV2 multivalent antibody that exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody.

In some aspects, the anti-SARS-CoV2 multivalent antibody exhibits improved prevention of immune escape compared to a monovalent anti-SARS-CoV2 antibody that binds to one of the same antigens as the anti-SARS-CoV2 multivalent antibody.

In some aspects, the anti-SARS-CoV2 multivalent antibody exhibits increased neutralization potency compared to a combination of monovalent anti-SARS-CoV2 antibodies that binds to the same antigens as the anti-SARS-CoV2 multivalent antibody.

In some aspects, the anti-SARS-CoV2 multispecific antibody prevents immune escape of the anti-SARS-CoV2 multivalent antibody for at least 2, at least 3, at least 4, at least 5, at least 6, or at least 7 selection rounds as measured by an antibody escape assay. In some aspects, the anti-SARS-CoV2 multispecific antibody prevents immune escape of the anti-SARS-CoV2 multivalent antibody for about 2 to about 7 selection rounds as measured by an antibody escape assay, or any value or range of values therein, for example, for about 3 to about 7 selection rounds, about 4 to about 7 selection rounds, about 5 to about 7 selection rounds, about 6 to about 7 selection rounds, about 2 to about 6 selection rounds, about 3 to about 6 selection rounds, about 4 to about 6 selection rounds, about 5 to about 6 selection rounds, about 2 to about 5 selection rounds, about 3 to about 5 selection rounds, about 4 to about 5 selection rounds, about 2 to about 4 selection rounds, about 3 to about 4 selection rounds, or about 2 to about 3 selection rounds as measured by an antibody escape assay. In some aspects, the anti-SARS-CoV2 multispecific antibody prevents immune escape of the anti-SARS-CoV2 multivalent antibody for about 2, about 3, about 4, about 5, about 6 or about 7 selection rounds as measured by an antibody escape assay.

In some aspects, the anti-SARS-CoV2 multispecific antibody prevents immune escape of the anti-SARS-CoV2 multivalent antibody following at least 2, at least 3, at least 4, at least 5, at least 6, or at least 7 exposures to the subject by the SARS-CoV2 virus. In some aspects, the anti-SARS-CoV2 multispecific antibody prevents immune escape of the anti-SARS-CoV2 multivalent antibody following about 2 to about 7 exposures to the subject by the SARS-CoV2 virus, or any value or range of values therein, for example, for about 3 to about 7 exposures, about 4 to about 7 exposures, about 5 to about 7 exposures, about 6 to about 7 exposures, about 2 to about 6 exposures, about 3 to about 6 exposures, about 4 to about 6 exposures, about 5 to about 6 exposures, about 2 to about 5 exposures, about 3 to about 5 exposures, about 4 to about 5 exposures, about 2 to about 4 exposures, about 3 to about 4 exposures, or about 2 to about 3 exposures to the subject by the SARS-CoV2 virus. In some aspects, the anti-SARS-CoV2 multispecific antibody prevents immune escape of the anti-SARS-CoV2 multivalent antibody following about 2, about 3, about 4, about 5, about 6 or about 7 exposures to the subject by the SARS-CoV2 virus.

In some aspects, the anti-SARS-CoV2 multispecific antibody is at least 100-fold, at least 500-fold, at least 1,000-fold, or at least 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, the anti-SARS-CoV2 multispecific antibody is from about 100-fold to about 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody, or any value or range of values therein, for example, from about 500-fold to about 2,000-fold, about 1,000-fold to about 2,000-fold, about 100-fold to about 1,000-fold, about 500-fold to about 1,000-fold, or about 100-fold to about 500-fold. In some aspects, the anti-SARS-CoV2 multispecific antibody is about 100-fold, about 500-fold, about 1,000-fold, or about 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody.

In some aspects, the anti-SARS-CoV2 multivalent antibody comprises a first antigen binding site that specifically binds to a SARS-CoV-2 protein and a second antigen binding site that specifically binds to a SARS-CoV-2 protein.

In some aspects, the first antigen binding site is different than the second antigen binding site.

In some aspects, the first antigen binding site comprises a first heavy chain variable domain and a first light chain variable domain, and the second antigen binding site comprises a second heavy chain variable domain and a second light chain variable domain.

In some aspects, the first and second heavy chain variable regions each comprise CDR1, CDR2 and CDR3 regions, and the first and second light chain variable region each comprise CDR1, CDR2 and CDR3 regions.

In some aspects, the CDR1 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176 184, 193, 201, 209, 217, 225, 233, 241, 249, 257, 265, 273, 281, 289, 297, 319, 327, 335 and 343; the CDR2 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; the CDR3 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345; the CDR1 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; the CDR2 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); the CDR3 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349; the CDR1 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; the CDR2 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; the CDR3 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195,203, 211, 219, 227, 235, 243, 251, 259, 267, 275, 283, 291, 299, 321, 329, 337 and 345; the CDR1 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; the CDR2 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and the CDR3 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349. In some preferred aspects, CDR1, CDR2 and CDR3 of each of the first heavy chain variable region, the first light chain variable region, the second heavy chain variable region and the second light chain variable region have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of the first heavy chain variable region, the first light chain variable region, the second heavy chain variable region and the second light chain variable region comprise or consist of a corresponding amino acid sequence specified above.

In some aspects, the first heavy chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; the first light chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346; the second heavy chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; and the second light chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:5, 13, 21, 29, 37, 151, 155, 163, 171 179, 188, 189, 191, 196, 204, 212, 220, 228, 236, 244, 252, 260, 268, 276, 284, 292, 300, 305, 307, 309, 311, 313, 315, 317, 322, 330, 338 and 346. In some aspects, the sequence has a mutation at N62, for example, N62Q or N62Q at, for example, SEQ ID NO:175. In some preferred aspects, each of the first heavy chain variable domain, the first light chain variable domain, the second heavy chain variable domain and the second light chain variable domain have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, each of the first heavy chain variable domain, the first light chain variable domain, the second heavy chain variable domain and the second light chain variable domain comprise or consist of a corresponding amino acid sequence specified above.

In some aspects, the anti-SARS-CoV2 multivalent antibody further comprises a third antigen binding domain.

In some aspects, the third antigen binding site is different than the first antigen binding site, the second antigen binding site, or both the first and second antigen binding sites.

In some aspects, the third antigen binding site comprises a third heavy chain variable domain and a third light chain variable domain.

In some aspects, the third heavy chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; and the third light chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346. In some aspects, the sequence has a mutation at N62, for example, N62Q or N62Q at, for example, SEQ ID NO:175. In some preferred aspects, the third heavy chain variable domain and the third light chain variable domain have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the third heavy chain variable domain and the third light chain variable domain comprise or consist of a corresponding amino acid sequence specified above.

In some aspects, the third heavy chain variable region comprises CDR1, CDR2 and CDR3 regions.

In some aspects, the CDR1 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; the CDR2 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; the CDR3 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345; the CDR1 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; the CDR2 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and the CDR3 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349. In some preferred aspects, CDR1, CDR2 and CDR3 of the third heavy chain variable region and the third light chain variable region have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of the third heavy chain variable region and the third light chain variable region comprise or consist of a corresponding amino acid sequence specified above.

In some aspects, the anti-SARS-CoV2 multivalent antibody further comprises a fourth antigen binding domain.

In some aspects, the fourth antigen binding site is different than one or more of the first, second and third antigen binding sites.

In some aspects, the first, second, third and fourth antigen binding sites are different.

In some aspects, the fourth antigen binding site comprises a third heavy chain variable domain and a fourth light chain variable domain.

In some aspects, the fourth heavy chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; and the fourth light chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346. In some aspects, the sequence has a mutation at N62, for example, N62Q or N62Q at, for example, SEQ ID NO:175. In some preferred aspects, the fourth heavy chain variable domain and the fourth light chain variable domain have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the fourth heavy chain variable domain and the fourth light chain variable domain comprise or consist of a corresponding amino acid sequence specified above.

In some aspects, the fourth heavy chain variable region comprises CDR1, CDR2 and CDR3 regions.

In some aspects, the CDR1 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; the CDR2 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; the CDR3 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345; the CDR1 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; the CDR2 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and the CDR3 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349. In some preferred aspects, CDR1, CDR2 and CDR3 of the fourth heavy chain variable region and the fourth light chain variable region have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of the fourth heavy chain variable region and the fourth light chain variable region comprise or consist of a corresponding amino acid sequence specified above.

In some aspects of any of the antigen binding polypeptides or antigen binding polypeptide complexes provided herein, one or more VL (e.g., VL1, VL2, VL3 and/or VL4) comprises an amino acid sequence having at least 80%, at least 85%, at least 90%, at least 95%, at least 99%, or 100% identity to a peptide in the expression vector of SEQ ID NO:355. In some aspects, such a polypeptide or polypeptide complex further comprises one or more VH (e.g., VH1, VH2, VH3 and/or VH4) that comprises an amino acid having at least 80%, at least 85%, at least 90%, at least 95%, at least 99%, or 100% identity to any one of SEQ ID NOs:175, 351, 353, 357 and 359.

In some aspects of any of the antigen binding polypeptides or antigen binding polypeptide complexes provided herein, one or more VL (e.g., VL1, VL2, VL3 and/or VL4) is encoded by a polynucleotide sequence having at least 80%, at least 85%, at least 90%, at least 95%, at least 99%, or 100% identity to SEQ ID NO:356. In some aspects, such a polypeptide or polypeptide complex further comprises one or more VH (e.g., VH1, VH2, VH3 and/or V14) that comprises an amino acid having at least 80%, at least 85%, at least 90%, at least 95%, at least 99%, or 100% identity to any one of SEQ ID NOs:175, 351, 353, 357 and 359.

In some aspects of any of the antigen binding polypeptides or antigen binding polypeptide complexes provided herein, one or more VH (e.g., VH1, VH2, VH3 and/or V14) is encoded by a polynucleotide sequence having at least 80%, at least 85%, at least 90%, at least 95%, at least 99%, or 100% identity to SEQ ID NO:350. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects of any of the antigen binding polypeptides or antigen binding polypeptide complexes provided herein, one or more VH (e.g., VH1, VH2, VH3 and/or V14) comprises an amino acid sequence having at least 80%, at least 85%, at least 90%, at least 95%, at least 99%, or 100% identity to SEQ ID NO:351. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects of any of the antigen binding polypeptides or antigen binding polypeptide complexes provided herein, one or more VH (e.g., VH1, VH2, VH3 and/or V14) is encoded by a polynucleotide having at least 80%, at least 85%, at least 90%, at least 95%, at least 99%, or 100% identity to SEQ ID NO:352. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects of any of the antigen binding polypeptides or antigen binding polypeptide complexes provided herein, one or more VH (e.g., VH1, VH2, VH3 and/or V14) comprises an amino acid sequence having at least 80%, at least 85%, at least 90%, at least 95%, at least 99%, or 100% identity to SEQ ID NO:353. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects of any of the antigen binding polypeptides or antigen binding polypeptide complexes provided herein, one or more VH (e.g., VH1, VH2, VH3 and/or V14) is encoded by a polynucleotide having at least 80%, at least 85%, at least 90%, at least 95%, at least 99%, or 100% identity to SEQ ID NO:354. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects of any of the antigen binding polypeptides or antigen binding polypeptide complexes provided herein, one or more VH (e.g., VH1, VH2, VH3 and/or V14) comprises an amino acid sequence having at least 80%, at least 85%, at least 90%, at least 95%, at least 99%, or 100% identity to a peptide in the expression vector of SEQ ID NO:357. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects of any of the antigen binding polypeptides or antigen binding polypeptide complexes provided herein, one or more VH (e.g., VH1, VH2, VH3 and/or V14) is encoded by a polynucleotide having at least 80%, at least 85%, at least 90%, at least 95%, at least 99%, or 100% identity to SEQ ID NO:358. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects of any of the antigen binding polypeptides or antigen binding polypeptide complexes provided herein, one or more VH (e.g., VH1, VH2, VH3 and/or V14) comprises an amino acid sequence having at least 80%, at least 85%, at least 90%, at least 95%, at least 99%, or 100% identity to a peptide in the expression vector of SEQ ID NO:359. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects of any of the antigen binding polypeptides or antigen binding polypeptide complexes provided herein, one or more VH (e.g., VH1, VH2, VH3 and/or V14) is encoded by a polynucleotide having at least 80%, at least 85%, at least 90%, at least 95%, at least 99%, or 100% identity to SEQ ID NO:360. In some aspects, such a polypeptide or polypeptide complex further comprises one or more VL (e.g., VL1, VL2, VL3 and/or VL4) that comprises an amino acid having at least 80%, at least 85%, at least 90%, at least 95%, at least 99%, or 100% identity to SEQ ID NO:171.

In some aspects, the antigen is the SARS-CoV-2 protein. In some aspects, the SARS-CoV-2 protein is a membrane protein, nucleocapsid protein, envelope protein, or spike protein. In some aspects, the SARS-CoV-2 protein is a spike protein.

In some aspects, the SARS-CoV2 virus is a SARS-CoV2 variant. In some aspects, the SARS-CoV2 variant is Alpha variant (B.1.1.7), Beta variant (B.1.351), Gamma variant (P.1), Delta variant, Epsilon variant (B.1.427), or Omicron variant (B.1.1.529), or a combination thereof.

In some aspects, the Omicron variant is BA. 1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof.

In some aspects, any VH and/or VL pair may be deglycosylated of high mannose sugars present on an amino acid or amino acids in the VH and/or VL sequence. Site specific mutation may be performed to replace the glycosylated amino acid with a deglycosylated amino acid substitute.

In some aspects, an antigen binding polypeptide or antigen binding polypeptide complex provided herein has a pI of from about 7 to about 9, from about 7 to about 8, or from about 8 to about 9. In some aspects, an antigen binding polypeptide or antigen binding polypeptide complex provided herein has a pI of about 7, about 7.5, about 8, about 8.5 or about 9. In some aspects, an antigen binding polypeptide or antigen binding polypeptide complex provided herein has a pI of about 6.92, about 6.99, about 6.73, about 8.36, about 7.8, or about 7.92. In some aspects, an antigen binding polypeptide or antigen binding polypeptide complex provided herein has a pI of at least about 7 or at least about 8.

In some aspects, an antigen binding polypeptide or antigen binding polypeptide complex provided herein does not precipitate.

In some aspects, an antigen binding polypeptide or antigen binding polypeptide complex provided herein has advantages over a monoclonal antibody or a combination of monoclonal antibodies binding to the same target as the antigen binding polypeptide or antigen binding polypeptide complex. For example, in some aspects, an antigen binding polypeptide or antigen binding polypeptide complex provided herein targets different sites on a SARS-CoV-2 protein. In some aspects, an antigen binding polypeptide or antigen binding polypeptide complex provided herein has synergistic neutralization of a SARS-CoV-2 virus. In some aspects, an antigen binding polypeptide or antigen binding polypeptide complex provided herein has cross coverage of antigenic variants of a SARS-CoV-2 virus. In some aspects, an antigen binding polypeptide or antigen binding polypeptide complex provided herein minimizes SARS-CoV-2 escape. In some aspects, an antigen binding polypeptide or antigen binding polypeptide complex provided herein remains therapeutically effective despite SARS-CoV-2 evolution.

### Amino Acid Linkers

In some aspects, an antigen binding polypeptide or polypeptide complex (e.g., an antibody or antigen binding fragment thereof) provided herein comprises one or more amino acid linkers between two or more regions of the antigen binding polypeptide or polypeptide complex.

As used herein, an “amino acid linker” can be a single amino acid or short amino acid sequence that is capable of joining two regions of an antigen binding polypeptide or polypeptide complex provided herein in a stable manner that maintains or promotes a function associated with the regions. However, as used herein, an “amino acid linker” can also include where an amino acid linker is not present between two regions (referred to herein as an amino acid linker having 0 amino acids). In some aspects, an amino acid linker is represented herein in a structure of an antigen binding polypeptide or polypeptide complex by the abbreviation “1” or “L” and a number (e.g., L1 to denote a first linker, L2 to denote a second linker, L3 to denote a third linker, L4 to denote a fourth linker, L5 to denote a fifth linker, L6 to denote a sixth linker, L7 to denote a seventh linker, and L8 to denote an eighth linker). In some aspects, such enumerated amino acid linkers (e.g., L1) can have the same or different sequence as any other enumerated amino acid linker (e.g., L2, etc.).

In some aspects, an amino acid linker has a length of from 0 amino acids (i.e., an amino acid linker is not present) to about 50 amino acids (e.g., one or more of L1, L2, L3, L4, L5, L6, L7 or L8 of a first, second, third and/or fourth polypeptide of an antigen binding polypeptide or polypeptide complex structure provided herein). In some aspects, the amino acid linker has a length of from 0 amino acids to about 45 amino acids, 0 amino acids to about 40 amino acids, 0 amino acids to about 35 amino acids, 0 amino acids to about 30 amino acids, 0 amino acids to about 25 amino acids, 0 amino acids to about 20 amino acids, 0 amino acids to about 15 amino acids, 0 amino acids to about 10 amino acids, 0 amino acids to about 5 amino acids, about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, 1 amino acid to about 15 amino acids, about 1 amino acid to about 10 amino acids, about 1 amino acid to about 5 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 50 amino acids, about 30 amino acids to about 45 amino acids, about 30 amino acids to about 40 amino acids, about 30 amino acids to about 35 amino acids, about 40 amino acids to about 50 amino acids, about 40 amino acids to about 45 amino acids, or about 45 amino acids to about 50 amino acids.

In some aspects, the amino acid linker has 0 amino acids (i.e., an amino acid linker is not present) or about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, or about 50 amino acids (e.g., one or more of L1, L2, L3, L4, L5, L6, L7 or L8 of a first, second, third and/or fourth polypeptide of an antigen binding polypeptide or polypeptide complex structure provided herein).

In some aspects, the amino acid linker consists of one or more amino acid residues (e.g., one or more of L1, L2, L3, L4, L5, L6, L7 or L8 of a first, second, third and/or fourth polypeptide of an antigen binding polypeptide or polypeptide complex structure provided herein). In some aspects, the amino acid residues are selected from the group consisting of glycine, alanine, serine, threonine, cysteine, asparagine, glutamine, leucine, isoleucine, valine, proline, histidine, aspartic acid, glutamic acid, lysine, arginine, methionine, phenylalanine, tryptophan, and tyrosine.

In some aspects, an amino acid linker is non-immunogenic. In some aspects, a non-immunogenic linker consists of serine, glycine and/or alanine residues, or consists of serine and/or glycine residues. In some aspects, an amino acid linker does not contain a T cell epitope or consensus T cell epitope.

In some aspects, an amino acid linker consists of one or more residues of alanine, cysteine, glycine, isoleucine, leucine, methionine, phenylalanine, proline, tryptophan, tyrosine, or valine (e.g., one or more of L1, L2, L3, L4, L5, L6, L7 or L8 of a first, second, third and/or fourth polypeptide of an antigen binding polypeptide or polypeptide complex structure provided herein).

Amino acid linker sequences that can be used with the antigen binding polypeptides or polypeptide complexes (e.g., an antibody or antigen binding fragment thereof) provided herein are well known and can be incorporated into the antigen binding polypeptides and polypeptide complexes provided herein using routine molecular biology and recombinant DNA techniques. See, e.g., Chen et al., Adv Drug Deliv Rev., 65(10):1357-1369, 2013; and Chichili et al., Protein Sci., 22(2):153-167, 2013.

In some aspects, an amino acid linker (e.g., one or more of L1, L2, L3, L4, L5, L6, L7 or L8 of a first, second, third and/or fourth polypeptide of an antigen binding polypeptide or polypeptide complex structure provided herein) comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, the amino acid linker comprises an amino acid sequence having at least 80% (such as at least 85%, at least 90%, at least 91%, at least 92%, at least 93%, at least 94%, at least 95%, at least 96%, at least 97%, at least 98%, at least 99% or 100%) identity to SEQ ID NO: 46.

In some aspects, L1 of the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L1 of the first polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L1 of the first polypeptide has 0 amino acids.

In some aspects, L2 of the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L2 of the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L2 of the second polypeptide has 0 amino acids.

In some aspects, L3 of the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L3 of the second polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L3 of the second polypeptide has 0 amino acids.

In some aspects, L3 of the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L3 of the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L3 of the third polypeptide has 0 amino acids.

In some aspects, L4 of the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L4 of the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L4 of the third polypeptide has 0 amino acids.

In some aspects, L5 of the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L5 of the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L5 of the third polypeptide has 0 amino acids.

In some aspects, L5 of the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L5 of the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L5 of the fourth polypeptide has 0 amino acids.

In some aspects, L6 of the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L6 of the third polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L6 of the third polypeptide has 0 amino acids.

In some aspects, L6 of the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L6 of the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L6 of the fourth polypeptide has 0 amino acids.

In some aspects, L7 of the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L7 of the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L7 of the fourth polypeptide has 0 amino acids.

In some aspects, L8 of the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:46-80 (wherein SEQ ID NO:47 has the sequence of G, SEQ ID NO:48 has the sequence of A, SEQ ID NO:49 has the sequence of GSS, and SEQ ID NO:50 has the sequence of ASG). In some aspects, L8 of the fourth polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to SEQ ID NO:46. In some aspects, L8 of the fourth polypeptide has 0 amino acids.

In some aspects, the SARS-CoV-2 of the SARS-CoV-2 protein to which an antigen binding polypeptide or polypeptide complex provided herein specifically binds is a variant of concern (VOC). In some aspects, the SARS-CoV-2 of the SARS-CoV-2 protein to which an antigen binding polypeptide or polypeptide complex provided herein specifically binds is a variant under monitoring (e.g., variant with genetic changes suspected to affect virus characteristics and some indication of posing a future risk, but further studies are needed).

In some aspects, the SARS-CoV-2 of the SARS-CoV-2 protein to which an antigen binding polypeptide or polypeptide complex provided herein specifically binds is a Lambda (C.37), Mu (B.1.621), Epsilon (B.1.429, B.1.427, CAL.20C), Zeta (P.2), Eta (B.1.525), Theta (P.3), Iota (B.1.526), or Kappa (B.1.617.1) variant, or a combination thereof. In some aspects, the SARS-CoV-2 of the SARS-CoV-2 protein to which an antigen binding polypeptide or polypeptide complex provided herein specifically binds is a AZ.5, C.1.2, B.1.617.1, B.1.526, B.1.525, B.1.630, B.1.640, AV.1, AT.1, R.1, B.1.466.2, B.1.1.519, C.36.3, B.1.214.2, B.1.427, B.1.429, B.1.1.523, B.1.619, B.1.620, B.1.1.207, B.1.1.317, B.1.616, B.1.618, or B.1.640.2 variant, or a combination thereof.

In some aspects, the SARS-CoV-2 of the SARS-CoV-2 protein to which an antigen binding polypeptide or polypeptide complex provided herein specifically binds is an Alpha (B.1.1.7), Beta (B.1.351), Gamma (P.1), Delta (B.1.617.2), or Omicron (B.1.1.529) variant, or a combination thereof.

In some aspects, the SARS-CoV-2 protein has an amino acid mutation of one or more of the following: F2L, L5F, L5I, V6F, L7V, P9L, S12C, S13I, Q14H, C15F, N17K, L18F, T20I, T22N, T22A, T22I, Q23K, P25S, A27S, A27V, T29I, F32L, R34C, H49Y, S50L, T51I, Q52L, Q52H, L54F, L54W, F55I, P57L, H69Y, S71F, G72V, T73I, G75V, T76I, F79L, D80N, D80Y, N87Y, D88N, D88E, D88Y, D88A, V90F, T95A, T95I, E96D, K97T, S98F, R1021, 1105L, D111N, K113R, L118F, V130A, E132D, C136R, D138H, L141-, L141F, G142V, G142-, V143F, V143-, Y144-, Y144V, Y145H, H146Y, K147E, N148S, S151I, W152C, M153T, M153V, M153I, E154V, F157L, R158S, L176F, M177I, D178N, G181V, L189F, R190K, I203M, I210-, R214L, D215Y, D215G, L216F, Q218L, F220L, S221L, A222V, A222P, D228H, L229F, Q239R, T240I, L242F, A243S, A243V, H245R, H245Y, R246K, D253Y, D253G, S254F, S256L, W258L, G261V, G261R, A262S, Y265C, V267L, R273S, E281Q, A288S, L293V, D294E, P295S, E298G, T307I, V308L, E309Q, Q314K, Q314L, Q314H, T315I, Q321L, T323I, P330S, A344S, T345S, A348T, A348S, N354K, R357K, V367F, V382L, P384L, V395I, R403K, V407I, A411S, G413R, K417T, N439K, N440K, L441I, G446V, R457K, K458Q, G476S, S477N, P479L, V483A, E484Q, E484K, Q493L, S494P, L452R, Y453F, Y508H, N501Y, H519Q, A520S, A522V, K529E, G545S, T547I, L552F, T553I, E554D, K558N, A570V, T572I, D574Y, E583D, I584V, S5961, 1598V, N603H, Q613H, D614G, V615F, T618A, P621S, V622F, V622I, V622A, A623S, H625Y, A626V, P631S, W633R, G639V, S640F, A647S, A647V, E654Z, E654K, H655Y, N658Y, A668S, A672V, Q675R, Q675H, T676S, T676I, Q677H, Q677R, T678I, P681L, P681R, P681H, R682W, A684S, A684T, V687L, A688V, A688S, S689I, S691F, S698L, N703D, S704L, V705F, A706V, I714M, T716I, I720V, T724A, M7311, T732A, T732I, G744V, D745G, N751D, L754F, R765S, R765H, T768I, G769A, A771S, T778I, Q779H, E780Q, A783S, D808V, D808G, P809S, I818V, L822F, D830H, D830Y, Q836P, Q836L, G838D, A845S, A845D, A845V, A845D, A845S, A846V, R847I, K854R, N856S, T859I, D867N, A879V, A879S, F888L, A893E, A893V, E918V, L922F, A924S, A924V, S929I, D936H, D936Y, L938F, S939F, T941I, G946V, A958S, N969S, L981F, T1006I, V1008T, T1009I, A1016S, A1020V, F1052L, P1053T, L1063F, V1065L, A1070V, Q1071H, E1072V, K1073N, A1078V, A1078S, G1085R, K1086N, R1091L, H1101Y, V1104L, P1112L, D1118Y, Ti 1201, V1122L, S1123P, G1124V, G1124C, V1129A, 11130M, T11361, D1139H, L1141F, D1146H, S1147L, D1153Y, P1162Q, P1162S, P1162Q, P1162S, D1163Y, G1167V, D1168H, V1176F, N1187Y, K1191N, N1192T, E1195Q, L1203F, K1205N, E1207A, 1219V, G1219S, I1221T, V1228L, M1229I, V1230L, T1231I, T1231A, C1235F, M1237I, M1237V, M1237T, T1238I, K1245N, C1247F, G1251R, D1259H, S1261F, P1263L, V1264L, a deletion of residues 69 and 70, a deletion of residues 246-252 and D253N, or a combination thereof.

### Modifications

In some aspects, an antigen binding polypeptide or polypeptide complex (e.g., an antibody or antigen binding fragment thereof) provided herein comprises an effector function mutation or half-life extension mutation.

Effector functions are an important part of the humoral immune response and form an link between innate and adaptive immunity. Most effector functions are induced via the Fc region of an antibody, which can interact with complement proteins and specialized Fc receptors. As used herein, an “effector function mutation” or “Fc effector function mutation” refer to a change in the amino acid sequence, typically in the Fc region, which can increase or decrease effector function, for example, increase binding affinity of Fc for specific Fc receptors, or increase antibody-dependent cellular cytotoxicity (ADCC) activity.

“Half-life” of a pharmaceutically active substance is the time it takes for the amount of the substance, once administered to the body, to reduce by half A “half-life extension mutation” of an antigen binding polypeptide or polypeptide complex provided herein refers to a change in the amino acid sequence, typically in the Fc region, which increases the half-life of the antigen binding polypeptide or polypeptide complex (e.g., by increasing Fc receptor binding affinity, slowing off-rate for Fc and Fc receptors, and/or increased sialylation).

Examples of Fc effector function mutations that decrease or knockout function include, but are not limited to, the following substitutions in the Fc region, based on the EU numbering scheme: L234A, L235A, P239A, N297A, or a combination thereof.

Examples of Fc effector function mutations that increase function include, but are not limited to, the following substitutions in the Fc region, based on the EU numbering scheme: S298A/E333A/K334A, S239D/I332E, S239D/A330L/I332E, G236A/S239D/I332E, or a combination thereof.

Additional examples of effector function mutations, half-life extension mutations and methods for incorporating the same into an amino acid sequence are known and described, for example, in Saunders, “Conceptual Approaches to Modulating Antibody Effector Functions and Circulation Half-Life,” Front. Immunol. Jun. 7, 2019.

In some aspects, an antigen binding polypeptide or polypeptide complex (e.g., an antibody or antigen binding fragment thereof) provided herein comprises one or more knob-into-hole modifications.

The term “knob-into-hole modification,” as used herein, refers to a genetic modification that directs the pairing of two polypeptides to promote heterodimerization. In some aspects, the modification introduces a protuberance (knob) into one polypeptide and a cavity (hole) into the other polypeptide at an interface in which the two polypeptides interact. In some aspects, a knob-into-hole modification can be created by introducing only a hole modification, for example, by replacing an amino acid residue with a smaller side chain than the original amino acid residue (e.g., a substitution of one or more serine, threonine, valine or alanine residues, or a combination thereof). In some aspects, a knob-into-hole modification can be created by introducing only a knob modification, for example, by replacing an amino acid residue with a larger side chain than the original amino acid residue (e.g., a substitution of one or more tryptophan or tyrosine residues, or a combination thereof).

In some aspects, the knob-into-hole modification is in the binding interface of two Fc regions, the binding interface of two CH2 regions, the binding interface of two CH3 regions, the binding interface of a CL region and a CH1 region, or the binding interface of a VH region and a VL region. See, e.g., U.S. Pub. No. 2007/0178552, Int'l Pub. No. WO 96/027011, Int'l Pub. No. WO 98/050431 and Zhu et al., Protein Science 6:781-788, 1987.

In some aspects, the antigen binding polypeptide or polypeptide complex comprises one, two, three, four, five, six, seven, eight, nine, ten, or more knob-into-hole modifications.

Knob-into-hole modifications are well known and can be incorporated into antigen binding polypeptides and polypeptide complexes provided herein using routine molecular biology and recombinant DNA techniques. See, e.g., U.S. Pub. No. 2003/0078385; Int'l Pub. No. WO 96/027011; Ridgway et al., Protein Eng., 9:617-621, 1996; and Merchant et al., Nat. Biotechnol., 16:677-681, 1998.

In some aspects, the knob-into-hole modification is an amino acid substitution. As used herein, such a substitution is described based on the EU numbering scheme of Kabat, which corresponds to the numbering in the Protein Data Bank (PDB).

In some aspects, the knob-into-hole modifications are M428L and N434S or N434A, based on the EU numbering scheme. In some aspects, the knob-into-hole modifications are M428L and N434S, based on the EU numbering scheme. In some aspects, the knob-into-hole modification is N434A, based on the EU numbering scheme.

In some aspects, the knob-into-hole modification is a knob substitution of S354C and/or T366W, based on the EU numbering scheme. In some aspects, the knob-into-hole modification is a knob substitution of S354C, T366W, M428L and N434S, based on the EU numbering scheme.

In some aspects, the knob-into-hole modification is a hole substitution of Y349C, T366S, L368A, Y407V, M428L, N434S, N434A, M252Y, S254T, T256E, or a combination thereof, based on the EU numbering scheme.

In some aspects, the knob-into-hole modifications are hole substitutions of Y349C, T366S, L368A and Y407V, based on the EU numbering scheme. In some aspects, the knob-into-hole modifications are hole substitutions of M428L and N434S or N434A, based on the EU numbering scheme. In some aspects, the knob-into-hole modifications are hole substitutions of M428L and N434S, based on the EU numbering scheme. In some aspects, the knob-into-hole modification is a hole substitution of N434A, based on the EU numbering scheme. In some aspects, the knob-into-hole modifications are hole substitutions of M252Y, S254T and T256E, based on the EU numbering scheme. In some aspects, the knob-into-hole modifications are hole substitutions of Y349C, T366S, L368A, Y407V, M428L and N434S.

In some aspects, an antigen binding polypeptide complex is an IgG1 or IgG4 antibody and the knob-into-hole modifications are knob substitutions of S354C and T366W and hole substitutions of Y349C, T366S, L368A and Y407V.

In some aspects, the antigen binding polypeptide complex is an IgG1 or IgG4 antibody and the knob-into-hole modifications are hole substitutions of M428L and N434S or N434A. In some aspects, the knob-into-hole modifications are hole substitutions of M428L and N434S. In some aspects, the knob-into-hole modification is a hole substitution of N434A.

In some aspects, the antigen binding polypeptide complex is an IgG1 or IgG4 antibody and the knob-into-hole modifications are hole substitutions of M252Y, S254T and T256E.

In some aspects, the modifications or mutations can be both in the Fc region and in the antigen binding polypeptide or antigen binding polypeptide complex.

### Detectable Labels

In some aspects, an antigen binding polypeptide or polypeptide complex (e.g., an antibody or antigen binding fragment thereof) provided herein comprises one or more detectable labels. An antigen binding polypeptide or polypeptide complex (e.g., an antibody or antigen binding fragment thereof) containing a detectable label is useful in therapeutic, diagnostic, imaging (e.g., radioimaging), or basic research applications.

In some aspects, the detectable label is a radioactive label. Examples of a radioactive label include, but are not limited to, the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc, ¹⁸⁶Re and ⁸⁹Zr.

In some aspects, the detectable label is a chemiluminescent label, fluorescent label, enzyme, biotin, or a combination thereof.

In some aspects, the detectable label is a peptide tag. In some aspects, the peptide tag is located at or near the N-terminus of the polypeptide or polypeptide complex. In some aspects, the peptide tag is located or near at the C-terminus of the polypeptide or polypeptide complex. In some aspects, the peptide tag is an affinity tag or fusion tag.

In some aspects, the detectable label is a polyhistidine tag, polyarginine tag, glutathione-S-transferase (GST), maltose binding protein (MBP), chitin binding protein (CBP), Strep-tag, thioredoxin (TRX), poly(NANP), FLAG tag, ALFA-tag, V5-tag, Myc-tag, hemagglutinin (HA) tag, Spot tag, T7 tag, NE tag, or green fluorescence protein (GFP), or a combination thereof. In some aspects, the polyhistidine tag consists of from about 4 to about 10 histidine residues. In some aspects, the polyhistidine tag consists of about 4, about 5, about 6, about 7, about 8, about 9, or about 10 histidine residues.

Additional examples of detectable labels and methods for introducing detectable labels into a polypeptide are known and include routine chemical, molecular biology and recombinant DNA techniques. See, e.g., Hnatowich et al., Science, 220(4597):613-615, 1983; Yao et al., Int. J. Mol. Sci., 17(2):194, 2016; Kimple et al., Curr. Protoc. Protein Sci., 73: Unit 9.9, 2013; Sambrook J, Fritsch E F. Molecular Cloning: A Laboratory Manual. Cold Spring Harbor Laboratory Press; Cold Spring Harbor, N.Y.: 1989; Molecular Cell Biology, 4^{th} edition, Section 3.5, Purifying, Detecting and Characterizing Proteins; and Mahmoodi et al., Cogent Biology, 5(1): DOI: 10/1080/23312025.2019.1665406.

### Polypeptides, Polynucleotides, Vectors, Cells, and Protein Production Methods

In some aspects, a polypeptide encoding an antigen binding polypeptide or antigen polypeptide complex (e.g., an antibody or antigen binding fragment thereof) is provided herein.

In some aspects, a polypeptide provided herein comprises one or more amino acid sequences having at least 80%, at least 85%, at least 90%, at least 95%, at least 99% or 100% identity to one or more of SEQ ID NOs:81-124. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 81. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 82. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:83. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO: 84. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:85. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:86. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:87. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:88. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:89. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:90. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:91. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:92. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:93. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:94. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:95. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:96. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:97. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:98. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:99. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:100. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:101. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:102. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:103. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:104. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:105. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:106. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:107. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:108. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:109. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:110. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:111. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:112. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:113. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:114. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:115. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:116. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:117. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:118. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:119. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:120. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:121. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:122. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:123. In some aspects, the polypeptide comprises or consists of an amino acid sequence having at least 80% (such as at least 85%, 90%, 91%, 92%, 93%, 94%, 95%, 96%, 97%, 98%, 99% or 100%) identity to SEQ ID NO:124.

In some aspects, a polypeptide provided herein comprises one or more amino acid sequences encoded by one or more polynucleotides having at least 80%, at least 85%, at least 90%, at least 91%, at least 92%, at least 93%, at least 94%, at least 95%, at least 96%, at least 97%, at least 98%, at least 99% or 100% identity to one or more of SEQ ID NOs:125-146. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO: 125. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:126. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:127. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO: 128. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:129. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:130. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:131. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:132. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:133. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:134. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:135. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:136. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:137. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:138. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:139. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:140. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:141. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO: 142. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:143. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:144. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:145. In some aspects, the polypeptide comprises or consists of an amino acid sequence encoded by a polynucleotide having at least 80% (such as at least 85%, 90%, 91%, 92%, 93%, 94%, 95%, 96%, 97%, 98%, 99% or 100%) identity to SEQ ID NO:146.

In some aspects, a polynucleotide provided herein comprises a sequence having at least 80%, at least 85%, at least 90%, at least 91%, at least 92%, at least 93%, at least 94%, at least 95%, at least 96%, at least 97%, at least 98%, at least 99% or 100% identity to one or more of SEQ ID NOs:125-146. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:125. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 126. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:127. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:128. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:129. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:130. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:131. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:132. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 134. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:135. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:136. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:137. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:138. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:139. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:140. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:141. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:142. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:143. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 144 In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO: 145. In some aspects, the polynucleotide comprises or consists of a polynucleotide sequence having at least 80% (such as at least 85%, 90%, 91%, 92%, 93%, 94%, 95%, 96%, 97%, 98%, 99% or 100%) identity to SEQ ID NO:146.

In some aspects, a polynucleotide provided herein encodes a polypeptide having at least 80%, at least 85%, at least 90%, at least 91%, at least 92%, at least 93%, at least 94%, at least 95%, at least 96%, at least 97%, at least 98%, at least 99% or 100% identity to one or more of SEQ ID NOs:81-124. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:81. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:82. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO: 83. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:84. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:85. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:86. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:87. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:88. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:89. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:90. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:91. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:92. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:93. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:94. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:95. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:96. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:97. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:98. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:99. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:100. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:101. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:102. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:103. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:104. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:105. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:106. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:107. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:108. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:109. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:110. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:111. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:112. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:113. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:114. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:115. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:116. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:117. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:118. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:119. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:120. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:121. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:122. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:123. In some aspects, the polynucleotide encodes a polypeptide having at least 80% (such as at least 85%, 90%, 91%, 92%, 93%, 94%, 95%, 96%, 97%, 98%, 99% or 100%) identity to SEQ ID NO:124.

In some aspects, provided herein is a vector comprising a polynucleotide provided herein.

In yet other aspects, provided herein is a host cell comprising a polynucleotide or vector provided herein.

As used herein, the term “host cell” can be any type of cell, e.g., a primary cell, a cell in culture, or a cell from a cell line. In some aspects, the term “host cell” refers to a cell containing a foreign gene [e.g., a cell subjected to gene delivery or transfected with a polynucleotide (e.g., DNA or mRNA) encoding the gene]transfected with a polynucleotide and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

Methods which are well known to those skilled in the art can be used to construct vectors encoding antigen binding polypeptides and polypeptide complexes (e.g., CDR, VH, VL, heavy chain and/or light chain coding sequences and appropriate transcriptional and translational control signals). These methods include, for example, in vitro recombinant DNA techniques, synthetic techniques, and in vivo genetic recombination.

A vector can be transferred to a host cell by conventional techniques and the resulting cell can then be cultured by conventional techniques to produce an antigen binding polypeptide or antigen binding polypeptide complex comprising, e.g., six CDRs, VH, VL, VH and VL, heavy chain, light chain, or heavy and light chain, or a domain thereof (e.g., one or more CDRs, VH, VL, VH and VL, heavy chain, or light chain). Thus, provided herein are host cells containing a polynucleotide encoding an antigen binding polypeptide or polypeptide complex comprising, e.g., comprising six CDRs, VH, VL, VH and VL, heavy chain, light chain, or heavy and light chain, or a domain thereof (e.g., one or more CDRs, VH, VL, VH and VL, heavy chain, or light chain), operably linked to a promoter for expression of such sequences in the host cell. In some aspects, vectors encoding both heavy and light chains, or a domain thereof, individually, can be co-expressed in the host cell for expression. In some aspects, a host cell contains a vector comprising a polynucleotide encoding both a heavy chain and light chain, or a domain thereof. In some aspects, a host cell contains two different vectors, a first vector comprising a polynucleotide encoding a heavy chain or a domain thereof, and a second vector comprising a polynucleotide encoding a light chain or a domain thereof. In some aspects, a first host cell comprises a first vector comprising a polynucleotide encoding a heavy chain or a domain thereof, and a second host cell comprises a second vector comprising a polynucleotide encoding a light chain or a domain thereof. In some aspects, provided herein is a population of host cells comprising such a first host cell and such a second host cell.

In some aspects, provided herein is a population of vectors comprising a first vector comprising a polynucleotide encoding a light chain or domain thereof, and a second vector comprising a polynucleotide encoding a heavy chain or domain thereof. Alternatively, a single vector can be used which encodes, and is capable of expressing, both heavy and light chain polypeptides or a domain thereof.

A variety of host-vector systems can be utilized to express the polypeptides and polypeptide complexes provided herein. Such host-vector systems represent vehicles by which the coding sequences of interest can be produced and subsequently purified, but also represent cells which can, when transformed or transfected with the appropriate nucleotide coding sequences, express a polypeptide or polypeptide complex provided herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH 3T3, HEK-293T, HepG2, SP210, R1.1, B-W, L-M, BSC1, BSC40, YB/20, and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing polypeptide or polypeptide complexes provided herein are CHO cells, for example CHO cells from the CHO GS System™ (Lonza). In some aspects, cells for expressing the polypeptides or polypeptide complexes provided herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells) are used for the expression of recombinant polypeptides. For example, mammalian cells such as Chinese hamster ovary (CHO) cells in conjunction with a vector such as the major intermediate early gene promoter element from human cytomegalovirus is an effective expression system for polypeptides (Foecking M K & Hofstetter H (1986) Gene 45: 101-105; and Cockett M I et al., (1990) Biotechnology 8: 662-667). In some aspects, the polypeptides or polypeptide complexes provided herein are produced by HEK-293T cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once a polypeptide or polypeptide complex provided herein has been produced by recombinant expression, it can be purified by any method known in the art for purification of a protein or immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the polypeptides or polypeptide complexes provided herein can be fused to heterologous polypeptide sequences provided herein (e.g., peptide tags) or otherwise known in the art to facilitate purification.

In some aspects, a polypeptide or polypeptide complex provided herein is isolated or purified. Generally, an isolated polypeptide or polypeptide complex is one that is substantially free of other polypeptides or polypeptide complexes with different antigenic specificities. For example, in some aspects, a preparation of a polypeptide or polypeptide complex provided herein is substantially free of cellular material and/or chemical precursors.

### Pharmaceutical Compositions and Kits

In some aspects, provided herein is a pharmaceutical composition comprising an antigen binding polypeptide complex (e.g., antibody or antigen binding fragment thereof), polypeptide, polynucleotide, vector, or host cell provided herein.

In some aspects, a pharmaceutical composition provided herein comprises (1) an antigen binding polypeptide complex (e.g., antibody or antigen binding fragment thereof), polypeptide, polynucleotide, vector, or host cell provided herein, and (2) a pharmaceutically acceptable carrier. The term “pharmaceutically acceptable carrier” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is known in the art. Except insofar as any conventional media or agent is incompatible with the active ingredient, its use in the therapeutic formulations is contemplated. Supplementary active ingredients can also be incorporated into the pharmaceutical compositions provided herein. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies. In some aspects, the pharmaceutical composition is for parenteral, intravenous or subcutaneous administration.

In some aspects, the present invention comprises a combination of a monoclonal antibody(ies) and an antigen binding polypeptide or antigen binding polypeptide complex which comprises a multispecific antibody.

In some aspects, the present invention comprises a nucleic acid encoding an antigen binding polypeptide or antigen binding polypeptide complex for in vivo administration. In some aspects, the nucleic acid encoding an antigen binding polypeptide or antigen binding polypeptide complex is a stabilized nucleic acid selected from a stabilized mRNA.

In some aspects, the nucleic acid encoding an antigen binding polypeptide or antigen binding polypeptide complex comprises said nucleic acid and a carrier to form a pharmaceutical composition.

In some aspects, a pharmaceutical composition provided herein comprises a lipid nanoparticle composition. As used herein, a “lipid nanoparticle composition,” “lipid nanoparticle formulation,” “LNP composition,” or “LNP formulation” is a composition comprising one or more lipids. The lipids in a lipid nanoparticle composition are typically sized on the order of micrometers or smaller. In some aspects, the lipids in a lipid nanoparticle composition have an average size of less than 1 micrometer.

In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid.

As used herein, a “cationic and/or ionizable lipid” is a lipid that has a positive or partial positive charge at physiological pH. Examples of a cationic and/or ionizable lipid include, but are not limited to, a lipid including a cyclic amine group, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S)-2-({8-[(30)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)), and mixtures thereof.

As used herein, a “structural lipid” is a fat synthesized from mixtures of long-chain and medium-chain fatty acids. A structural lipid is typically a triacylglycerol restructured or modified to change the fatty acid composition and/or positional distribution. Examples of a structural lipid include, but are not limited to, cholesterol, fecosterol, sitosterol, ergosterol, campesterol, stigmasterol, brassicasterol, tomatidine, tomatine, ursolic acid, alpha-tocopherol, and mixtures thereof.

As used herein, a “PEGylated lipid” is a lipid that has been modified to include polyethylene glycol or “PEG.” Examples of a PEGylated lipid include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, PEG-c-DOMG, PEG-DMG, PEG-DLPE, PEG-DMPE, PEG-DPPC, a PEG-DSPE lipid, and mixtures thereof.

As used herein, a “phospholipid” is a class of lipids whose molecule has a hydrophilic “head” containing a phosphate group and two hydrophobic “tails” derived from fatty acids, joined by an alcohol residue (usually a glycerol molecule). Examples of a phospholipid include, but are not limited to, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, the phospholipid is DSPC. In some aspects, the phospholipid is DOPE. In some aspects, the phospholipid is DSPC and DOPE.

Exemplary lipid nanoparticle compositions and methods for making them are described, for example, in U.S. Pat. Nos. 11,524,023; 11,485,972; 10,898,574; 10,703,789; 10,702,600; 10,577,403; 10,442,756; 10,266,485; 10,064,959; 9,868,692; Int'l Pub. No. WO 2019/046809; Int'l Pub. No. WO 2020/160397; U.S. Pub. No. 2020/0306191; Xu et al., Adv. Nanobio. Res. 2:2, 2022; Cullis et al., Mol. Ther. 25(7):1467-1475, 2017; Fan et al., J. Pharm. Biomed. Anal. 192:113642, 2020; Paunovska et al., Nature Rev., 23:265-280, 2022; Buck et al., ACS Nano. 13:3754-3782, 2019; Pardi et al., Nature Comm. 8:14630, 2017; and Pardi et al., J. Control Release, 217:345-351, 2015, which are incorporated by reference herein. Such methods include, but are not limited to, back translating DNA coding for an antigen binding polypeptide or antigen binding polypeptide complex provided herein (e.g., an antibody or antigen binding fragment thereof) into an in vitro transcription (IVT) plasmid according to such published methods.

Exemplary compositions and methods related to generation and delivery of mRNA are described, for example, herein and in Int'l Pub. No. WO 2018/081638, Int'l Pub. No. WO 2017/182524, U.S. Pat. No. 9,012,219, Int'l Pub. No. WO 2016/176330, U.S. Pat. No. 9,371,511, U.S. Appl. Pub. No. 2018/0028645, U.S. Appl. Pub. No. 23018/0344838, U.S. Pub. No. 2018/0265848, U.S. Appl. Pub. No. 2017/0043037, U.S. Appl. Pub. No. 2017/0327842, U.S. Pat. No. 10,006,007, U.S. Appl. Pub. No. 2013/0261172, U.S. Appl. Pub. No. 2013/0197068, U.S. Appl. Pub. No. 2015/0038558, U.S. Appl. Pub. No. 2016/0032316, U.S. Appl. Pub. No. 2013/0111615, U.S. Appl. Pub. No. 2009/0286852, Cullis et al., Mol. Ther. 25(7):1467-1475, 2017; Pardi et al., Nature Comm. 8:14630, 2017; Pardi et al., J. Control Release, 217:345-351, 2015; and Grier et al., Mol. Ther. Nucleic Acids, 5:e306 (2016), which are incorporated by reference herein. Such methods include, but are not limited to, in vitro transcription (e.g., using PCR products or a linearized plasmid); IVT vectors; and synthesizing mRNA using, e.g., HiScribe T7 High Yield RNA Synthesis Kit (New England Biolabs), incorporating, e.g., Pseudouridine-5′-Triphosphate (TriLink BioTechnologies). In some aspects, 5′ capped mRNA can also be generated during this process using, e.g., CleanCap Reagent AG (TriLink BioTechnologies) to form, e.g., 100 bp or 120 bp poly(A) tails.

In some aspects, provided herein is a pharmaceutical composition comprising one or more antigen binding polypeptide complexes provided herein (e.g., 1, 2, 3, 4, 5 or more). In some aspects, the pharmaceutical composition further comprises a pharmaceutically acceptable carrier. In some aspects, one or more of the antigen binding polypeptide complexes is an antibody or antigen binding fragment thereof provided herein.

In some aspects, provided herein is a pharmaceutical composition comprising one or more antigen binding polypeptide complexes provided herein (e.g., 1, 2, 3, 4, 5 or more) and an additional pharmaceutical agent. In some aspects, the pharmaceutical composition further comprises a pharmaceutically acceptable carrier. In some aspects, one or more of the antigen binding polypeptide complexes is an antibody or antigen binding fragment thereof provided herein.

In some aspects, the additional pharmaceutical agent is 25-hydroxyvitamin D, an agent that potentiates vitamin D action, an anti-viral agent, an anti-malarial agent, an antibiotic, or a combination thereof.

In some aspects, the additional pharmaceutical agent is 25-hydroxyvitamin D.

In some aspects, the agent that potentiates vitamin D action is a CYP24 inhibitor, 1,25-dihydroxyvitamin D compound, or a combination thereof.

In some aspects, the anti-viral agent is an anti-retroviral agent, an antibody against SARS-CoV-2 virus, an inhibitor of reverse transcriptase, or a combination thereof.

In some aspects, the anti-viral agent is maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, sofobuvir, bebtelovimab (LY1404, LY-CoV1404, LY3853133), or a combination thereof.

In some aspects, the anti-viral agent is bebtelovimab.

In some aspects, the anti-viral agent is Retrovir® (3′-azido-3′-deoxypyrimidine, zidovudine), 3′-azido-3′-deoxythymidine (AZT), HMD® (2′,3′-dideoxycytidine, zalcitabine), VidexEC® (2′,3′dideoxyinosine, didanosine), Epivir® (lamivudine), Zerit® (stavudine), Viread® (tenofovir DF), Ziagen® (abacavir), Emtriva® (emtricitabine, FTC), Rescriptor® (delavirdine), Sustiva® (efavirenz), Viramune® (nevirapine, 11-cyclopropyl-4-methyl-5,11-dihydro-6H-dipyrido[3,2-b:2′,3′-e][1,4]diazepin-6-one), trisodium phosphonoformate, ammonium-21-tungstenato-9-antimonate, 1-β-D-ribofuranoxyl-1,2,4-triazole-3-carboxamide, Aganerase® (amprenavir), Reyataz® (atazanavir), Lexiva® (fosamprenavir), Crixivan® (indinavir), Viracept® (nelfinavir), Norvir® (ritonavir), Fortovase® or Invirase® (saquinavir), lasinavir (5(S)-(tert-butoxycarbonylamino)-4(S)-hydroxy-6-phenyl-2(R)(2,3,4-trimethoxyphenylmethyl)-hexanoyl-(L)-valyl-N-(2-metoxy-ethyl)-amide), adriamycin, KVX-478, VX-478, 141W94, AG-1343, KNI-272, U-96988, BILA-2011 BS (palinavir), polymannoacetate, Fuzeon® (enfuvirtide, T-20), Epzicom® (abacavir and lamivudine), Trizivir® (abacavir, lamivudine and zidovudine), Truvada® (emtricitabine and tenofir DF), Combivir® (lamivudine and zidovudine), Kaletra® (lopinavir and ritonavir), or a combination thereof.

In some aspects, a pharmaceutical composition provided herein further comprises a pharmaceutically acceptable carrier.

In some aspects, provided herein is a kit comprising an antigen binding polypeptide complex (e.g., antibody or antigen binding fragment thereof), polypeptide, polynucleotide, vector, host cell, or pharmaceutical composition provided herein, or a combination thereof. Once a pharmaceutical composition has been formulated, it can be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. In some aspects, provided herein is a kit for producing a single-dose administration unit. In some aspects, the kit contains a first container having a dried antigen binding polypeptide or polypeptide complex provided herein (e.g., antibody or antigen binding fragment thereof) and a second container having an aqueous formulation. In some aspects, provided herein is a kit containing an antigen binding polypeptide or polypeptide complex provided herein (e.g., antibody or antigen binding fragment thereof) in a single and/or multi-chambered pre-filled syringe (e.g., liquid syringe and/or lyosyringe). In some aspects, the kit further contains components for intravenous or subcutaneous administration.

In some aspects, a kit provided herein comprises one or more antigen binding polypeptide complexes (e.g., antibodies or antigen binding fragments thereof) provided herein (e.g., 1, 2, 3, 4, 5 or more). In some aspects, a kit provided herein comprises an antigen binding polypeptide complexes (e.g., antibodies or antigen binding fragments thereof) provided herein.

In some aspects, a kit provided herein comprises one or more pharmaceutical compositions comprising an antigen binding polypeptide complex (e.g., antibody or antigen binding fragment thereof) provided herein (e.g., 1, 2, 3, 4, 5 or more). In some aspects, a kit provided herein comprises a pharmaceutical compositions comprising an antigen binding polypeptide complexes (e.g., antibody or antigen binding fragment thereof) provided herein. In some aspects, the pharmaceutical composition further comprises a pharmaceutically acceptable carrier.

In some aspects, a kit provided herein comprises (i) one or more antigen binding polypeptide complexes (e.g., antibodies or antigen binding fragments thereof) provided herein (e.g., 1, 2, 3, 4, 5 or more), and (ii) one or more additional pharmaceutical agents (e.g., 1, 2, 3, 4, 5 or more). In some aspects, a kit provided herein comprises (i) an antigen binding polypeptide complex (e.g., antibody or antigen binding fragment thereof) provided herein, and (ii) an additional pharmaceutical agent.

In some aspects, a kit provided herein comprises (i) one or more pharmaceutical compositions comprising an antigen binding polypeptide complex (e.g., antibody or antigen binding fragment thereof) provided herein (e.g., 1, 2, 3, 4, 5 or more), and (ii) one or more additional pharmaceutical agents (e.g., 1, 2, 3, 4, 5 or more). In some aspects, a kit provided herein comprises (i) a pharmaceutical composition comprising an antigen binding polypeptide complex (e.g., antibody or antigen binding fragment thereof) provided herein, and (ii) an additional pharmaceutical agent. In some aspects, the pharmaceutical composition further comprises a pharmaceutically acceptable carrier.

In some aspects, the kit further comprises instructions for use, for example, for any of the methods of use described herein (e.g., treating or preventing a SARS-CoV-2 infection).

### Methods of Use

In some aspects, provided herein are certain methods of use of an antigen binding polypeptide complex (e.g., an antibody or antigen binding fragment thereof), polypeptide, polynucleotide, vector, host cell, or pharmaceutical composition provided herein, or a combination thereof. In some aspects, the antigen binding polypeptide complex (e.g., an antibody or antigen binding fragment thereof), polypeptide, polynucleotide, vector, host cell, or pharmaceutical composition provided herein neutralizes multiple SARS-CoV-2 variants. In some aspects, the antigen binding polypeptide complex (e.g., an antibody or antigen binding fragment thereof), polypeptide, polynucleotide, vector, host cell, or pharmaceutical composition provided herein neutralizes diverse SARS-CoV-2 variants. In some aspects, the antigen binding polypeptide complex (e.g., an antibody or antigen binding fragment thereof), polypeptide, polynucleotide, vector, host cell, or pharmaceutical composition provided herein provides broad coverage against SARS-CoV-2 variants. Any of the antigen binding polypeptide complex (e.g., an antibody or antigen binding fragment thereof), polypeptide, polynucleotide, vector, host cell, or pharmaceutical composition described herein may be used in any of the methods and uses of the invention.

In some aspects, provided herein is a method of preventing or treating a SARS-CoV-2 infection, comprising administering an antigen binding polypeptide complex (e.g., an antibody or antigen binding fragment thereof), polypeptide, polynucleotide, vector, host cell or pharmaceutical composition provided herein, or a combination thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in treating or preventing a SARS-CoV-2 infection. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of a SARS-CoV-2 infection. In some aspects, preventing or treating a SARS-CoV-2 infection comprises administering a pharmaceutical composition comprising (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of preventing or treating a SARS-CoV-2 infection in a subject in need thereof, comprising administering to the subject an effective amount of an antigen binding polypeptide complex (e.g., an antibody or antigen binding fragment thereof), polypeptide, polynucleotide, vector, host cell or pharmaceutical composition provided herein, or a combination thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in treating or preventing a SARS-CoV-2 infection in a subject in need thereof. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of a SARS-CoV-2 infection in a subject in need thereof. In some aspects, preventing or treating a SARS-CoV-2 infection in a subject in need thereof comprises administering a pharmaceutical composition comprising (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of preventing or treating coronavirus disease 2019 (COVID-19), comprising administering an antigen binding polypeptide complex (e.g., an antibody or antigen binding fragment thereof), polypeptide, polynucleotide, vector, host cell or pharmaceutical composition, or a combination thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in treating or preventing coronavirus disease 2019 (COVID-19). In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of coronavirus disease 2019 (COVID-19). In some aspects, preventing or treating coronavirus disease 2019 (COVID-19) comprises administering a pharmaceutical composition comprising (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of preventing or treating COVID-19 in a subject in need thereof, comprising administering to the subject an effective amount of an antigen binding polypeptide complex (e.g., an antibody or antigen binding fragment thereof), polypeptide, polynucleotide, vector, host cell or pharmaceutical composition, or a combination thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in treating or preventing COVID-19 in a subject in need thereof. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for the treatment or prevention of COVID-19 in a subject in need thereof. In some aspects, preventing or treating COVID-19 comprises administering a pharmaceutical composition comprising (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of preventing immune escape of a SARS-CoV-2 variant, comprising administering an antigen binding polypeptide complex (e.g., an antibody or antigen binding fragment thereof), polypeptide, polynucleotide, vector, host cell or pharmaceutical composition provided herein, or a combination thereof. In some aspects, provided herein is a method of preventing immune escape of a SARS-CoV-2 variant in a subject in need thereof, comprising administering to the subject an effective amount of an antigen binding polypeptide complex (e.g., an antibody or antigen binding fragment thereof), polypeptide, polynucleotide, vector, host cell or pharmaceutical composition provided herein, or a combination thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in a method of preventing immune escape of a SARS-CoV-2 variant in a subject in need thereof. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for preventing immune escape of a SARS-CoV-2 variant in a subject in need thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof prevents immune escape of the SARS-CoV-2 variant in the subject. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof prevents immune escape of the SARS-CoV-2 variant in the subject. In some aspects, the variant is an Omicron variant. In some aspects, the Omicron variant is BA1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1 or a combination thereof. In some aspects, preventing immune escape of a SARS-CoV-2 variant comprises administering a pharmaceutical composition comprising (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of neutralizing a SARS-CoV-2 variant, comprising administering an antigen binding polypeptide complex (e.g., an antibody or antigen binding fragment thereof), polypeptide, polynucleotide, vector, host cell or pharmaceutical composition provided herein, or a combination thereof. In some aspects, provided herein is a method of neutralizing a SARS-CoV-2 variant in a subject in need thereof, comprising administering to the subject an effective amount of an antigen binding polypeptide complex (e.g., an antibody or antigen binding fragment thereof), polypeptide, polynucleotide, vector, host cell or pharmaceutical composition provided herein, or a combination thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in a method of neutralizing a SARS-CoV-2 variant in a subject in need thereof. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for neutralizing a SARS-CoV-2 variant in a subject in need thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in preventing or treating infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof neutralizes the SARS-CoV-2 variant in the subject. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof neutralizes the SARS-CoV-2 variant in the subject. In some aspects, multiple SARS-CoV-2 variants are neutralized. In some aspects, diverse SARS-CoV-2 variants are neutralized. In some aspects, treating or preventing a SARS-CoV-2 infection by neutralizing a SARS-CoV-2 variant comprises administering a pharmaceutical composition comprising (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of increasing neutralization potency against a SARS-CoV-2 variant, comprising administering an antigen binding polypeptide complex (e.g., an antibody or antigen binding fragment thereof), polypeptide, polynucleotide, vector, host cell or pharmaceutical composition provided herein, or a combination thereof. In some aspects, provided herein is a method of increasing neutralization potency against a SARS-CoV-2 variant in a subject in need thereof, comprising administering to the subject an effective amount of an antigen binding polypeptide complex (e.g., an antibody or antigen binding fragment thereof), polypeptide, polynucleotide, vector, host cell or pharmaceutical composition provided herein, or a combination thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in a method of increasing neutralization potency against a SARS-CoV-2 variant in a subject in need thereof. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for increasing neutralization potency against a SARS-CoV-2 variant in a subject in need thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof increases neutralization potency against the SARS-CoV-2 variant in the subject. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for treating or preventing infection with a SARS-CoV-2 variant in a subject in need thereof, wherein the antigen binding polypeptide complex (e.g., antibody or antigen binding fragment thereof), polypeptide, polynucleotide, vector, cell, pharmaceutical composition or combination thereof increases neutralization potency against the SARS-CoV-2 variant in the subject. In some aspects, the increased neutralization potency is relative to a monospecific antigen binding polypeptide complex that specifically binds to one of the same antigens as the antigen binding polypeptide complex. In some aspects, the increased neutralization potency is relative to a mixture of monospecific antigen binding polypeptide complexes that specifically binds to the same antigens as the antigen binding polypeptide complex. In some aspects, the variant is an Omicron variant. In some aspects, the Omicron variant is BA1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof. In some aspects, increasing neutralization potency against a SARS-CoV-2 variant comprises administering a pharmaceutical composition comprising (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, provided herein is a method of increasing neutralization potency against multiple SARS-CoV-2 variants, comprising administering an antigen binding polypeptide complex (e.g., an antibody or antigen binding fragment thereof), polypeptide, polynucleotide, vector, host cell or pharmaceutical composition provided herein, or a combination thereof. In some aspects, provided herein is a method of increasing neutralization potency against multiple SARS-CoV-2 variants in a subject in need thereof, comprising administering to the subject an effective amount of an antigen binding polypeptide complex (e.g., an antibody or antigen binding fragment thereof), polypeptide, polynucleotide, vector, host cell or pharmaceutical composition provided herein, or a combination thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in a method of increasing neutralization potency against multiple SARS-CoV-2 variants in a subject in need thereof. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for increasing neutralization potency against multiple SARS-CoV-2 variants in a subject in need thereof. In some aspects, provided herein is an antigen binding polypeptide complex (e.g., antibody or antigen binding fragment thereof), polypeptide, polynucleotide, vector, cell, or pharmaceutical composition described herein, or a combination thereof, for use in treating or preventing infection with multiple SARS-CoV-2 variants in a subject in need thereof, wherein the antigen binding polypeptide complex (e.g., antibody or antigen binding fragment thereof), polypeptide, polynucleotide, vector, cell, pharmaceutical composition or combination thereof increases neutralization potency against multiple SARS-CoV-2 variants in the subject. In some aspects, provided herein is the use of an antigen binding polypeptide complex (e.g., antibody or antigen binding fragment thereof), polypeptide, polynucleotide, vector, cell, or pharmaceutical composition described herein, or a combination thereof in the manufacture of a medicament for treating or preventing infection with multiple SARS-CoV-2 variants in a subject in need thereof, wherein the antigen binding polypeptide complex (e.g., antibody or antigen binding fragment thereof), polypeptide, polynucleotide, vector, cell, pharmaceutical composition or combination thereof increases neutralization potency against multiple SARS-CoV-2 variants in the subject. In some aspects, the increased neutralization potency is relative to a monospecific antigen binding polypeptide complex that specifically binds to one of the same antigens as the antigen binding polypeptide complex. In some aspects, the increased neutralization potency is relative to a mixture of monospecific antigen binding polypeptide complexes that specifically bind to the same antigens as the antigen binding polypeptide complex. In some aspects, the variants are Omicron variants. In some aspects, the Omicron variants are BA1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof. In some aspects, increasing neutralization potency against multiple SARS-CoV-2 variants comprises administering a pharmaceutical composition comprising (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

As used herein, the terms “prevent” or “preventing” refer to the prevention of the onset, recurrence or spread, in whole or in part, of a disease or condition provided herein, or a symptom thereof.

As used herein, the terms “treat” or “treating” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treat” can also mean prolonging survival as compared to expected survival if not receiving treatment.

As used herein, “administering” is meant a method of giving a dosage of an antigen binding polypeptide or polypeptide complex (e.g., antibody or antigen binding fragment thereof) to a subject in need thereof (e.g., a patient). Administering can be by any suitable means, including parenteral, intrapulmonary or intranasal. Parenteral infusions include, for example, intramuscular, intravenous, intraarterial, intraperitoneal or subcutaneous administration. Dosing can be by any suitable route, e.g., by injection, such as intravenous or subcutaneous injection. Various dosing schedules including, but not limited to, single or multiple administrations over various time-points, bolus administration, and pulse infusion are contemplated herein.

As used herein, an “effective amount” is an amount of an antigen binding polypeptide or antigen binding polypeptide complex (e.g., an antibody or antigen binding fragment thereof) that is sufficient to achieve the desired effect and can vary according to the nature and severity of the condition, and the potency of the polypeptide or polypeptide complex. In some aspects, the effective amount is from about 5 μg/mL to about 100 μg/mL, or any value or range of values therein (in some aspects, e.g., over about 30 days post-infusion). In some aspects, the effective amount is from about 5 μg/mL to about 75 μg/mL, from about 5 μg/mL to about 50 μg/mL, from about 5 μg/mL to about 25 μg/mL, from about 5 μg/mL to about 10 μg/mL, from about 10 μg/mL to about 100 μg/mL, from about 10 μg/mL to about 75 μg/mL, from about 10 μg/mL to about 50 μg/mL, from about 10 μg/mL to about 25 μg/mL, from about 25 μg/mL to about 100 μg/mL, from about 25 μg/mL to about 75 μg/mL, from about 25 μg/mL to about 50 μg/mL, from about 50 μg/mL to about 100 μg/mL, from about 50 μg/mL to about 75 μg/mL, or from about 75 μg/mL to about 100 μg/mL. In some aspects, the effective amount is about 5 μg/mL, about 10 μg/mL, about 25 μg/mL, about 50 μg/mL, about 75 μg/mL, or about 100 μg/mL.

In some aspects, an antigen binding polypeptide or polypeptide complex can be delivered by administering a polynucleotide, vector, or host cell that encodes the antigen binding polypeptide or polypeptide complex. In some aspects, an antigen binding polypeptide or polypeptide complex thereof can be delivered by administering a pharmaceutical composition containing the polypeptide or polypeptide complex. A therapeutic effect is the relief, to at least some extent, of one or more symptoms of the disease or disorder, and can include curing a disease or disorder. “Curing” means that the symptoms of active disease are eliminated. However, certain long-term or permanent effects of a disease or disorder can exist even after a cure is obtained.

As used herein, the term “subject” means a human or a non-human mammal, e.g., a dog, cat, mouse, rat, cow, sheep, pig, goat, non-human primate or bird, e.g., chicken, as well as any other vertebrate or invertebrate. In some aspects, the subject is a human. In some aspects, the subject is a veterinary animal. In some aspects, the subject is a mammal.

In some aspects, provided herein is a method of diagnosing a subject as being infected with a SARS-CoV-2 virus or suspected of being infected with a SARS-CoV-2 virus, comprising (i) contacting a sample obtained from the subject with the antigen binding polypeptide or polypeptide complex (e.g., an antibody or antigen binding fragment thereof) provided herein; (ii) detecting the presence or absence of a virus complex which contains the polypeptide or polypeptide complex and a SARS-CoV-2 virus, virion or fragment thereof; and (iii) diagnosing the subject as being infected with a SARS-CoV-2 virus or suspected of being infected with a SARS-CoV-2 virus when the presence of the virus complex is detected. In some aspects, the method is performed in vitro or ex vivo.

In some aspects, provided herein is a method of diagnosing a subject as not being infected with a SARS-CoV-2 virus or not suspected of being infected with a SARS-CoV-2 virus, comprising (i) contacting a sample obtained from the subject with the antigen binding polypeptide or polypeptide complex (e.g., an antibody or antigen binding fragment thereof) provided herein; (ii) detecting the presence or absence of a virus complex which contains the polypeptide or polypeptide complex and a SARS-CoV-2 virus, virion or fragment thereof, and (iii) diagnosing the subject as not being infected with a SARS-CoV-2 virus or not being suspected of being infected with a SARS-CoV-2 virus when the presence of the virus complex is not detected. In some aspects, the method is performed in vitro or ex vivo.

In some aspects, provided herein is a method of diagnosing a subject as having COVID-19 or suspected of having COVID-19, comprising (i) contacting a sample obtained from the subject with an antigen binding polypeptide or polypeptide complex (e.g., an antibody or antigen binding fragment thereof) provided herein; (ii) detecting the presence or absence of a virus complex which contains the polypeptide or polypeptide complex and a SARS-CoV-2 virus, virion or fragment thereof, and (iii) diagnosing the subject as having COVID-19 or suspected or having COVID-19 when the presence of the virus complex is detected. In some aspects, the method is performed in vitro or ex vivo.

In some aspects, provided herein is a method of diagnosing a subject as not having COVID-19 or not suspected of having COVID-19, comprising (i) contacting a sample obtained from the subject with an antigen binding polypeptide or polypeptide complex (e.g., an antibody or antigen binding fragment thereof) provided herein; (ii) detecting the presence or absence of a virus complex which contains the polypeptide or polypeptide complex and a SARS-CoV-2 virus, virion or fragment thereof; and (iii) diagnosing the subject as not having COVID-19 or not suspected of having COVID-19 when the presence of the virus complex is not detected. In some aspects, the method is performed in vitro or ex vivo.

The term “sample,” as used herein, refers to a composition that is obtained or derived from a subject that contains a cellular and/or other molecular entity that is to be characterized and/or identified, for example, based on physical, biochemical, chemical, and/or physiological characteristics. A sample includes, but is not limited to, nasal fluid or discharge (e.g., from a nasal swab), tissue, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, tissue culture medium, tissue extracts such as homogenized tissue, cellular extracts, and combinations thereof.

Methods for collecting, processing and storing a sample are known, and described, for example, in Vaught et al., IARC Sci. Pub., Unit 2. Chapter 3, pp. 23-42, 2011; CDC Interim Guidelines for Collecting and Handling of Clinical Specimens for COVID-19 Testing, Oct. 25, 2021; and Minghetti et al., Recommendations for collection, transport and storage of COVID-19 biological samples, ISS working group on Translational Research COVID-19, Istituto Superiore di Sanita, Apr. 15, 2020.

Methods for detecting the binding of an antigen binding polypeptide or polypeptide complex (e.g., antibody or antigen binding fragment thereof) provided herein and a SARS-CoV-2 virus, virion or fragment thereof are also known. Such methods include, but are not limited to, immunosorbent assay (ELISA), sodium dodecyl sulfate-polyacrylamide gel electrophoresis (SDS-PAGE), immunospot assay, lateral flow assay, flow cytometry, immunohistochemistry or western blot. See, e.g., Kumar et al., VirusDisease, 31:97-105, 2020; Gong et al., Front. Mol. Biosci. Jul. 23, 2021; and Espejo et al., Am. J. Clin. Pathol., 154(3)293-304, 2020.

In some aspects, the disclosure provides a method of delivering an antigen binding polypeptide or antigen binding polypeptide complex provided herein. In some aspects, the method includes administering to a subject a pharmaceutical composition provided herein. In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, the disclosure provides a method of producing an antigen binding polypeptide or antigen binding polypeptide complex provided herein in a cell. In some aspects, the method includes administering to a subject a pharmaceutical composition provided herein. In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, the disclosure provides a method of treating a disease or disorder provided herein. In some aspects, the method includes administering to a subject a pharmaceutical composition provided herein. In some aspects, the pharmaceutical composition comprises (1) one or more polynucleotides encoding an antigen binding polypeptide or antigen binding polypeptide complex provided herein, and (2) a lipid nanoparticle composition. In some aspects, the lipid nanoparticle composition comprises one or more of (i) a cationic and/or ionizable lipid, (ii) a structural lipid, (iii) a PEGylated lipid, and (iv) a phospholipid, as provided herein.

In some aspects, the SARS-CoV-2 of the SARS-CoV-2 protein to which an antigen binding polypeptide or polypeptide complex provided herein specifically binds is a variant of concern (VOC). In some aspects, the SARS-CoV-2 of the SARS-CoV-2 protein to which an antigen binding polypeptide or polypeptide complex provided herein specifically binds is a variant under monitoring (e.g., variant with genetic changes suspected to affect virus characteristics and some indication of posing a future risk, but further studies are needed).

In some aspects, the SARS-CoV-2 of the SARS-CoV-2 protein to which an antigen binding polypeptide or polypeptide complex provided herein specifically binds is a Lambda (C.37), Mu (B.1.621), Epsilon (B.1.429, B.1.427, CAL.20C), Zeta (P.2), Eta (B.1.525), Theta (P.3), Iota (B.1.526), or Kappa (B.1.617.1) variant, or a combination thereof. In some aspects, the SARS-CoV-2 of the SARS-CoV-2 protein to which an antigen binding polypeptide or polypeptide complex provided herein specifically binds is a AZ.5, C.1.2, B.1.617.1, B.1.526, B.1.525, B.1.630, B.1.640, AV.1, AT.1, R.1, B.1.466.2, B.1.1.519, C.36.3, B.1.214.2, B.1.427, B.1.429, B.1.1.523, B.1.619, B.1.620, B.1.1.207, B.1.1.317, B.1.616, B.1.618, or B.1.640.2 variant, or a combination thereof.

In some aspects, the SARS-CoV-2 of the SARS-CoV-2 protein to which an antigen binding polypeptide or polypeptide complex provided herein specifically binds is an Alpha (B.1.1.7), Beta (B.1.351), Gamma (P.1), Delta (B.1.617.2), or Omicron (B.1.1.529) variant, or a combination thereof.

In some aspects, the spike protein has an amino acid mutation of one or more of the following: F2L, L5F, L5I, V6F, L7V, P9L, S12C, S13I, Q14H, C15F, N17K, L18F, T20I, T22N, T22A, T22I, Q23K, P25S, A27S, A27V, T29I, F32L, R34C, H49Y, S50L, T51I, Q52L, Q52H, L54F, L54W, F55I, P57L, H69Y, S71F, G72V, T73I, G75V, T76I, F79L, D80N, D80Y, N87Y, D88N, D88E, D88Y, D88A, V90F, T95A, T95I, E96D, K97T, S98F, R1021, 1105L, D111N, K113R, L118F, V130A, E132D, C136R, D138H, L141-, L141F, G142V, G142-, V143F, V143-, Y144-, Y144V, Y145H, H146Y, K147E, N148S, S151I, W152C, M153T, M153V, M153I, E154V, F157L, R158S, L176F, M177I, D178N, G181V, L189F, R190K, I203M, I210-, R214L, D215Y, D215G, L216F, Q218L, F220L, S221L, A222V, A222P, D228H, L229F, Q239R, T240I, L242F, A243S, A243V, H245R, H245Y, R246K, D253Y, D253G, S254F, S256L, W258L, G261V, G261R, A262S, Y265C, V267L, R273S, E281Q, A288S, L293V, D294E, P295S, E298G, T307I, V308L, E309Q, Q314K, Q314L, Q314H, T315I, Q321L, T323I, P330S, A344S, T345S, A348T, A348S, N354K, R357K, V367F, V382L, P384L, V395I, R403K, V407I, A411S, G413R, K417T, N439K, N440K, L441I, G446V, R457K, K458Q, G476S, S477N, P479L, V483A, E484Q, E484K, Q493L, S494P, L452R, Y453F, Y508H, N501Y, H519Q, A520S, A522V, K529E, G545S, T547I, L552F, T553I, E554D, K558N, A570V, T572I, D574Y, E583D, I584V, S5961, 1598V, N603H, Q613H, D614G, V615F, T618A, P621S, V622F, V622I, V622A, A623S, H625Y, A626V, P631S, W633R, G639V, S640F, A647S, A647V, E654Z, E654K, H655Y, N658Y, A668S, A672V, Q675R, Q675H, T676S, T676I, Q677H, Q677R, T678I, P681L, P681R, P681H, R682W, A684S, A684T, V687L, A688V, A688S, S689I, S691F, S698L, N703D, S704L, V705F, A706V, I714M, T716I, I720V, T724A, M7311, T732A, T732I, G744V, D745G, N751D, L754F, R765S, R765H, T768I, G769A, A771S, T778I, Q779H, E780Q, A783S, D808V, D808G, P809S, I818V, L822F, D830H, D830Y, Q836P, Q836L, G838D, A845S, A845D, A845V, A845D, A845S, A846V, R847I, K854R, N856S, T859I, D867N, A879V, A879S, F888L, A893E, A893V, E918V, L922F, A924S, A924V, S929I, D936H, D936Y, L938F, S939F, T941I, G946V, A958S, N969S, L981F, T1006I, V1008T, T1009I, A1016S, A1020V, F1052L, P1053T, L1063F, V1065L, A1070V, Q1071H, E1072V, K1073N, A1078V, A1078S, G1085R, K1086N, R1091L, H1101Y, V1104L, P1112L, D1118Y, T11201, V1122L, S1123P, G1124V, G1124C, V1129A, 11130M, T11361, D1139H, L1141F, D1146H, S1147L, D1153Y, P1162Q, P1162S, P1162Q, P1162S, D1163Y, G1167V, D1168H, V1176F, N1187Y, K1191N, N1192T, E1195Q, L1203F, K1205N, E1207A, 1219V, G1219S, I1221T, V1228L, M1229I, V1230L, T12311, T1231A, C1235F, M1237I, M1237V, M1237T, T1238I, K1245N, C1247F, G1251R, D1259H, S1261F, P1263L, V1264L, a deletion of residues 69 and 70, a deletion of residues 246-252 and D253N, or a combination thereof.

In some aspects, the antigen binding polypeptides and antigen binding polypeptide complexes provided herein potently inhibit one or more SARS-CoV-2 virus variants.

In some aspects, provided herein is a method of preventing immune escape of a SARS-CoV2 virus in a subject infected with the SARS-CoV2 virus, comprising administering to the subject an effective amount of an antigen binding polypeptide or polypeptide complex provided herein, an antibody or antigen binding fragment thereof provided herein, a pharmaceutical composition provided herein, or a combination thereof. In some aspects, provided herein is an antigen binding polypeptide or polypeptide complex, antibody or antigen binding fragment thereof, or pharmaceutical composition described herein, or a combination thereof, for use in a method of preventing immune escape of a SARS-CoV-2 virus in a subject infected with the SARS-CoV-2 virus. In some aspects, provided herein is the use of an antigen binding polypeptide or polypeptide complex, antibody or antigen binding fragment thereof, or pharmaceutical composition described herein, or a combination thereof, in the manufacture of a medicament for preventing immune escape of a SARS-CoV-2 virus in a subject infected with the SARS-CoV-2 virus. In some aspects, provided herein is an antigen binding polypeptide or polypeptide complex, antibody or antigen binding fragment thereof, or pharmaceutical composition described herein, or a combination thereof for use in preventing or treating a SARS-CoV-2 virus infection in a subject in need thereof, wherein the antigen binding polypeptide or polypeptide complex, antibody or antigen binding fragment thereof, pharmaceutical composition or combination thereof prevents immune escape of the SARS-CoV-2 virus in the subject. In some aspects, provided herein is the use of an antigen binding polypeptide or polypeptide complex, antibody or antigen binding fragment thereof, or pharmaceutical composition described herein, or a combination thereof, in the manufacture of a medicament for preventing or treating a SARS-CoV-2 virus infection in a subject in need thereof, wherein the antigen binding polypeptide or polypeptide complex, antibody or antigen binding fragment thereof, pharmaceutical composition or combination thereof prevents immune escape of the SARS-CoV-2 virus in the subject.

In some aspects, provided herein is a method of preventing immune escape of a SARS-CoV2 virus in a subject infected with the SARS-CoV2 virus, comprising administering to the subject an effective amount of an anti-SARS-CoV2 multivalent antibody that exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody. In some aspects, provided herein is an anti-SARS-CoV2 multivalent antibody for use in a method of preventing immune escape of a SARS-CoV2 virus in a subject infected with the SARS-CoV2 virus, wherein the anti-SARS-CoV2 multivalent antibody exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody. In some aspects, provided herein is the use of an anti-SARS-CoV2 multivalent antibody in the manufacture of a medicament for preventing immune escape of a SARS-CoV2 virus in a subject infected with the SARS-CoV2 virus, wherein the anti-SARS-CoV2 multivalent antibody exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody. In some aspects, provided herein is an anti-SARS-CoV2 multivalent antibody for use in preventing or treating a SARS-CoV-2 virus infection in a subject in need thereof, wherein the anti-SARS-CoV2 multivalent antibody prevents immune escape of the SARS-CoV-2 virus in the subject, and wherein the anti-SARS-CoV2 multivalent antibody exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody. In some aspects, provided herein is the use of an anti-SARS-CoV2 multivalent antibody in the manufacture of a medicament for preventing or treating a SARS-CoV-2 virus infection in a subject in need thereof, wherein the anti-SARS-CoV2 multivalent antibody prevents immune escape of the SARS-CoV-2 virus in the subject, and wherein the anti-SARS-CoV2 multivalent antibody exhibits improved prevention of immune escape compared to (i) a monovalent anti-SARS-CoV2 antibody that binds to one of the same antigens as the anti-SARS-CoV2 multivalent antibody, and/or (ii) a combination of monovalent anti-SARS-CoV2 antibodies that binds to the same antigens as the anti-SARS-CoV2 multivalent antibody.

In some aspects, provided herein is a method of increasing neutralization potency against a SARS-CoV-2 virus in a subject infected with the SARS-CoV2 virus, comprising administering to the subject an effective amount of an antigen binding polypeptide or polypeptide complex provided herein, an antibody or antigen binding fragment thereof provided herein, or a combination thereof. In some aspects, provided herein is an antigen binding polypeptide or polypeptide complex, an antibody or antigen binding fragment thereof provided herein, or a combination thereof for use in a method of increasing neutralization potency against a SARS-CoV-2 virus in a subject infected with the SARS-CoV2 virus. In some aspects, provided herein is the use of an antigen binding polypeptide or polypeptide complex, an antibody or antigen binding fragment thereof provided herein, or a combination thereof in the manufacture of a medicament for increasing neutralization potency against a SARS-CoV-2 virus in a subject infected with the SARS-CoV2 virus. In some aspects, provided herein is an antigen binding polypeptide or polypeptide complex, an antibody or antigen binding fragment thereof provided herein, or a combination thereof for use in preventing or treating a SARS-CoV-2 virus in a subject, wherein the antigen binding polypeptide or polypeptide complex, antibody or antigen binding fragment thereof, or combination thereof increases neutralization potency against the SARS-CoV-2 virus in the subject. In some aspects, provided herein is the use of an antigen binding polypeptide or polypeptide complex, an antibody or antigen binding fragment thereof provided herein, or a combination thereof in the manufacture of a medicament for preventing or treating a SARS-CoV-2 virus infection in a subject in need thereof, wherein the antigen binding polypeptide or polypeptide complex, antibody or antigen binding fragment thereof, or combination thereof increases neutralization potency against the SARS-CoV-2 virus in the subject.

In some aspects, provided herein is a method of increasing neutralization potency against a SARS-CoV-2 virus in a subject infected with the SARS-CoV2 virus, comprising administering to the subject an effective amount of an anti-SARS-CoV2 multivalent antibody that exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, provided herein is an anti-SARS-CoV2 multivalent antibody for use in a method of increasing neutralization potency against a SARS-CoV-2 virus in a subject infected with the SARS-CoV2 virus, wherein the anti-SARS-CoV2 multivalent antibody exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, provided herein is the use of an anti-SARS-CoV2 multivalent antibody in the manufacture of a medicament for increasing neutralization potency against a SARS-CoV-2 virus in a subject infected with the SARS-CoV2 virus, wherein the anti-SARS-CoV2 multivalent antibody exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, provided herein is an anti-SARS-CoV2 multivalent antibody for use in preventing or treating a SARS-CoV-2 virus infection in a subject in need thereof, wherein the anti-SARS-CoV2 multivalent antibody increases neutralization potency against the SARS-CoV-2 virus in the subject, and wherein the anti-SARS-CoV2 multivalent antibody exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, provided herein is the use of an anti-SARS-CoV2 multivalent antibody in the manufacture of a medicament for preventing or treating a SARS-CoV-2 virus infection in a subject in need thereof, wherein the anti-SARS-CoV2 multivalent antibody increases neutralization potency against the SARS-CoV-2 virus in the subject, and wherein the anti-SARS-CoV2 multivalent antibody exhibits increased neutralization potency compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody.

In some aspects, the anti-SARS-CoV2 multivalent antibody is administered in a pharmaceutical composition. In some aspects, the pharmaceutical composition further comprises a pharmaceutically acceptable carrier.

In some aspects, the anti-SARS-CoV2 multivalent antibody exhibits improved prevention of immune escape compared to a monovalent anti-SARS-CoV2 antibody that binds to one of the same antigens as the anti-SARS-CoV2 multivalent antibody.

In some aspects, the anti-SARS-CoV2 multivalent antibody exhibits increased neutralization potency compared to a combination of monovalent anti-SARS-CoV2 antibodies that binds to the same antigens as the anti-SARS-CoV2 multivalent antibody.

In some aspects, the anti-SARS-CoV2 multispecific antibody prevents immune escape of the anti-SARS-CoV2 multivalent antibody for at least 2, at least 3, at least 4, at least 5, at least 6, or at least 7 selection rounds as measured by an antibody escape assay. In some aspects, the anti-SARS-CoV2 multispecific antibody prevents immune escape of the anti-SARS-CoV2 multivalent antibody for about 2 to about 7 selection rounds as measured by an antibody escape assay, or any value or range of values therein, for example, for about 3 to about 7 selection rounds, about 4 to about 7 selection rounds, about 5 to about 7 selection rounds, about 6 to about 7 selection rounds, about 2 to about 6 selection rounds, about 3 to about 6 selection rounds, about 4 to about 6 selection rounds, about 5 to about 6 selection rounds, about 2 to about 5 selection rounds, about 3 to about 5 selection rounds, about 4 to about 5 selection rounds, about 2 to about 4 selection rounds, about 3 to about 4 selection rounds, or about 2 to about 3 selection rounds as measured by an antibody escape assay. In some aspects, the anti-SARS-CoV2 multispecific antibody prevents immune escape of the anti-SARS-CoV2 multivalent antibody for about 2, about 3, about 4, about 5, about 6 or about 7 selection rounds as measured by an antibody escape assay.

In some aspects, the anti-SARS-CoV2 multispecific antibody prevents immune escape of the anti-SARS-CoV2 multivalent antibody following at least 2, at least 3, at least 4, at least 5, at least 6, or at least 7 exposures to the subject by the SARS-CoV2 virus. In some aspects, the anti-SARS-CoV2 multispecific antibody prevents immune escape of the anti-SARS-CoV2 multivalent antibody following about 2 to about 7 exposures to the subject by the SARS-CoV2 virus, or any value or range of values therein, for example, for about 3 to about 7 exposures, about 4 to about 7 exposures, about 5 to about 7 exposures, about 6 to about 7 exposures, about 2 to about 6 exposures, about 3 to about 6 exposures, about 4 to about 6 exposures, about 5 to about 6 exposures, about 2 to about 5 exposures, about 3 to about 5 exposures, about 4 to about 5 exposures, about 2 to about 4 exposures, about 3 to about 4 exposures, or about 2 to about 3 exposures to the subject by the SARS-CoV2 virus. In some aspects, the anti-SARS-CoV2 multispecific antibody prevents immune escape of the anti-SARS-CoV2 multivalent antibody following about 2, about 3, about 4, about 5, about 6 or about 7 exposures to the subject by the SARS-CoV2 virus.

In some aspects, the anti-SARS-CoV2 multispecific antibody is at least 100-fold, at least 500-fold, at least 1,000-fold, or at least 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody. In some aspects, the anti-SARS-CoV2 multispecific antibody is from about 100-fold to about 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody, or any value or range of values therein, for example, from about 500-fold to about 2,000-fold, about 1,000-fold to about 2,000-fold, about 100-fold to about 1,000-fold, about 500-fold to about 1,000-fold, or about 100-fold to about 500-fold. In some aspects, the anti-SARS-CoV2 multispecific antibody is about 100-fold, about 500-fold, about 1,000-fold, or about 2,000-fold more effective in inhibiting growth of the SARS-CoV2 virus compared to (i) a monovalent anti-SARS-CoV2 antibody that binds to one of the same antigens as the anti-SARS-CoV2 multivalent antibody and/or (ii) a combination of monovalent anti-SARS-CoV2 antibodies that binds to the same antigens as the anti-SARS-CoV2 multivalent antibody In some aspects, the anti-SARS-CoV2 multivalent antibody comprises a first antigen binding site that specifically binds to a SARS-CoV-2 protein and a second antigen binding site that specifically binds to a SARS-CoV-2 protein.

In some aspects, the first antigen binding site is different than the second antigen binding site.

In some aspects, the first antigen binding site comprises a first heavy chain variable domain and a first light chain variable domain, and the second antigen binding site comprises a second heavy chain variable domain and a second light chain variable domain.

In some aspects, the first and second heavy chain variable regions each comprise CDR1, CDR2 and CDR3 regions, and the first and second light chain variable region each comprise CDR1, CDR2 and CDR3 regions.

In some aspects, the CDR1 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; the CDR2 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; the CDR3 of the first heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345; the CDR1 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; the CDR2 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); the CDR3 of the first light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349; the CDR1 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; the CDR2 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; the CDR3 of the second heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195,203,211,219,227,235,243,251,259,267,275,283,291, 299, 321, 329, 337 and 345; the CDR1 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; the CDR2 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and the CDR3 of the second light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349. In some preferred aspects, CDR1, CDR2 and CDR3 of each of the first heavy chain variable region, the first light chain variable region, the second heavy chain variable region and the second light chain variable region have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of each of the first heavy chain variable domain, the first light chain variable domain, the second heavy chain variable domain and the second light chain variable domain comprise or consist of a corresponding amino acid sequence specified above.

In some aspects, the first heavy chain variable domain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; the first light chain variable domain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346; the second heavy chain variable domain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; and the second light chain variable domain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:5, 13, 21, 29, 37, 151, 155, 163, 171 179, 188, 189, 191, 196, 204, 212, 220, 228, 236, 244, 252, 260, 268, 276, 284, 292, 300, 305, 307, 309, 311, 313, 315, 317, 322, 330, 338 and 346. In some aspects, the sequence has a mutation at N62, for example, N62Q or N62Q at, for example, SEQ ID NO:175. In some preferred aspects, each of the first heavy chain variable domain, the first light chain variable domain, the second heavy chain variable domain and the second light chain variable domain have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, each of the first heavy chain variable domain, the first light chain variable domain, the second heavy chain variable domain and the second light chain variable domain comprise or consist of a corresponding amino acid sequence specified above.

In some aspects, the anti-SARS-CoV2 multivalent antibody further comprises a third antigen binding domain.

In some aspects, the third antigen binding site is different than the first antigen binding site, the second antigen binding site, or both the first and second antigen binding sites.

In some aspects, the third antigen binding site comprises a third heavy chain variable domain and a third light chain variable domain.

In some aspects, the third heavy chain variable domain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; and the third light chain variable domain comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346. In some aspects, the sequence has a mutation at N62, for example, N62Q or N62Q at, for example, SEQ ID NO:175. In some preferred aspects, the third heavy chain variable domain and the third light chain variable domain have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the third heavy chain variable domain and the third light chain variable domain comprise or consist of a corresponding amino acid sequence specified above.

In some aspects, the third heavy chain variable region comprises CDR1, CDR2 and CDR3 regions.

In some aspects, the CDR1 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; the CDR2 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; the CDR3 of the third heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345; the CDR1 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; the CDR2 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and the CDR3 of the third light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349. In some preferred aspects, CDR1, CDR2 and CDR3 of the third heavy chain variable region and the third light chain variable region have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of the third heavy chain variable region and the third light chain variable region comprise or consist of a corresponding amino acid sequence specified above.

In some aspects, the anti-SARS-CoV2 multivalent antibody further comprises a fourth antigen binding domain.

In some aspects, the fourth antigen binding site is different than one or more of the first, second and third antigen binding sites.

In some aspects, the first, second, third and fourth antigen binding sites are different.

In some aspects, the fourth antigen binding site comprises a third heavy chain variable domain and a fourth light chain variable domain.

In some aspects, the fourth heavy chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:1, 9, 17, 25, 33, 147, 159, 167, 175, 183, 190, 192, 200, 208, 216, 224, 232, 240, 248, 256, 264, 272, 280, 288, 296, 304, 306, 308, 310, 312, 314, 316, 318, 326, 334, 342, 351, 353, 357 and 359; and the fourth light chain variable domain comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs:5, 13, 21, 29, 37, 151, 155, 163, 171, 179, 188, 189, 191, 196, 204, 212, 220, 228, 236, 244, 252, 260, 268, 276, 284, 292, 300, 305, 307, 309, 311, 313, 315, 317, 322, 330, 338 and 346. In some aspects, the sequence has a mutation at N62, for example, N62Q or N62Q at, for example, SEQ ID NO:175. In some preferred aspects, the fourth heavy chain variable domain and the fourth light chain variable domain have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, the fourth heavy chain variable domain and the fourth light chain variable domain comprise or consist of a corresponding amino acid sequence specified above.

In some aspects, the fourth heavy chain variable region comprises CDR1, CDR2 and CDR3 regions.

In some aspects, the CDR1 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:2, 10, 18, 26, 34, 148, 160, 168, 176, 184, 193, 201, 209, 217, 225, 233, 241, 249, 257, 265, 273, 281, 289, 297, 319, 327, 335 and 343; the CDR2 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:3, 11, 19, 27, 35, 149, 161, 169, 177, 185, 194, 202, 210, 218, 226, 234, 242, 250, 258, 266, 274, 282, 290, 298, 320, 328, 336 and 344; the CDR3 of the fourth heavy chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:4, 12, 20, 28, 36, 150, 162, 170, 178, 186, 195, 203, 211, 219, 227, 235, 243, 251, 259, 267, 275, 283, 291, 299, 321, 329, 337 and 345; the CDR1 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:6, 14, 22, 30, 38, 152, 156, 164, 172, 180, 197, 205, 213, 221, 229, 237, 245, 253, 261, 269, 277, 285, 293, 301, 323, 331, 339 and 347; the CDR2 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:7 (having the sequence of SAS), 15 (having the sequence of GTN), 23 (having the sequence of GAS), 31 (having the sequence of DAS), and 39 (having the sequence of GAS), 153, 157, 165 (having the sequence of GAS), 173 (having the sequence of LGS), 181, 198 (having the sequence of DAS), 206 (having the sequence of DVS), 214 (having the sequence of EDS), 222 (having the sequence of KDS), 230 (having the sequence of DAS), 238 (having the sequence of AAS), 246 (having the sequence of GAS), 254 (having the sequence of DDS), 262 (having the sequence of KDS), 270 (having the sequence of SAS), 278 (having the sequence of SAS), 286 (having the sequence of GAS), 294 (having the sequence of GAS), 302 (having the sequence of GAS), 324 (having the sequence of GAS), 332 (having the sequence of DAS), 340 (having the sequence of SAS) and 348 (having the sequence of GAS); and the CDR3 of the fourth light chain variable region comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to any one of SEQ ID NOs:8, 16, 24, 32, 40, 154, 158, 166, 174, 182, 199, 207, 215, 223, 231, 239, 247, 255, 263, 271, 279, 287, 295, 303, 325, 333, 341 and 349. In some preferred aspects, CDR1, CDR2 and CDR3 of the fourth heavy chain variable region and the fourth light chain variable region have at least 90% identity with a corresponding amino acid sequence specified above. As used herein, “at least 90% identity” includes at least 91%, 92%, 93%, 94%, 95%, 96%, 97%, 98%, 99% and 100% identity to the recited reference sequence. In some particularly preferred aspects, CDR1, CDR2 and CDR3 of the fourth heavy chain variable region and the fourth light chain variable region comprise or consist of a corresponding amino acid sequence specified above.

In some aspects, the anti-SARS-CoV2 multivalent antibody is any antibody or antigen binding fragment thereof described herein.

In some aspects, the antigen is the SARS-CoV-2 protein. In some aspects, the SARS-CoV-2 protein is a membrane protein, nucleocapsid protein, envelope protein, or spike protein. In some aspects, the SARS-CoV-2 protein is a spike protein.

In some aspects, the SARS-CoV2 virus is a SARS-CoV2 variant. In some aspects, the SARS-CoV2 variant is Alpha variant (B.1.1.7), Beta variant (B.1.351), Gamma variant (P.1), Delta variant, Epsilon variant (B.1.427), or Omicron variant (B.1.1.529), or a combination thereof.

In some aspects, the Omicron variant is BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7, CH.1.1, or a combination thereof.

In some aspects, the method further comprises administering to the subject an effective amount of one or more additional pharmaceutical agents (e.g., 1, 2, 3, 4, 5 or more). In some aspects, the method further comprises administering to the subject an effective amount of an additional pharmaceutical agent. In some aspects, treating or preventing a disease or infection as disclosed herein further comprises administering to the subject an effective amount of one or more additional pharmaceutical agents (e.g., 1, 2, 3, 4, 5 or more). In some aspects, treating or preventing a disease or infection as disclosed herein further comprises administering to the subject an effective amount of an additional pharmaceutical agent.

In some aspects, the method further comprises administering to the subject an effective amount of one or more pharmaceutical compositions comprising (i) an additional pharmaceutical agent and (ii) a pharmaceutically acceptable carrier (e.g., 1, 2, 3, 4, 5 or more). In some aspects, the method further comprises administering to the subject an effective amount of a pharmaceutical composition comprising (i) an additional pharmaceutical agent and (ii) a pharmaceutically acceptable carrier. In some aspects, treating or preventing a disease or infection as disclosed herein further comprises administering to the subject an effective amount of one or more pharmaceutical compositions comprising (i) an additional pharmaceutical agent and (ii) a pharmaceutically acceptable carrier (e.g., 1, 2, 3, 4, 5 or more). In some aspects, treating or preventing a disease or infection as disclosed herein further comprises administering to the subject an effective amount of a pharmaceutical composition comprising (i) an additional pharmaceutical agent and (ii) a pharmaceutically acceptable carrier.

In some aspects, the additional pharmaceutical agent is 25-hydroxyvitamin D, an agent that potentiates vitamin D action, an anti-viral agent, an anti-malarial agent, an antibiotic, or a combination thereof.

In some aspects, the additional pharmaceutical agent is 25-hydroxyvitamin D.

In some aspects, the agent that potentiates vitamin D action is a CYP24 inhibitor, 1,25-dihydroxyvitamin D compound, or a combination thereof.

In some aspects, the anti-viral agent is an anti-retroviral agent, an antibody against SARS-CoV-2 virus, an inhibitor of reverse transcriptase, or a combination thereof.

In some aspects, the anti-viral agent is maraviroc, enfuvirtide, amantadine, lamivudine, nevirapine, efavirenz, dolutegravir, elvitegravir, raltegravir, acyclovir and any nucleoside analog of aciclovir, ganciclovir, cidofovir, forcarnet, ribavirin, interferon alpha, pegylated interferon alpha, boceprevir, atazanavir, darunavir, indinavir, oseltamivir, zanamivir, rimantadine, peremivir, valaciclovir, penciclovir, valganciclovir, foscarnet, tenofovir, adefovir, entecavir, lamivudine, telbivudine, ribavirin, glecaprevir, grazoprevir, paritaprevir, simeprevir, voxilaprevir, daclatasvir, elbasvir, ledipasvir, ombitasvir, pibrentasvir, velpatasvir, dasabuvir, famciclovir, remdesivir, trifluridine, sofobuvir, bebtelovimab (LY1404, LY-CoV1404, LY3853133), or a combination thereof.

In some aspects, the anti-viral agent is bebtelovimab.

In some aspects, the anti-viral agent is Retrovir® (3′-azido-3′-deoxypyrimidine, zidovudine), 3′-azido-3′-deoxythymidine (AZT), HMD® (2′,3′-dideoxycytidine, zalcitabine), VidexEC® (2′,3′dideoxyinosine, didanosine), Epivir® (lamivudine), Zerit® (stavudine), Viread® (tenofovir DF), Ziagen® (abacavir), Emtriva® (emtricitabine, FTC), Rescriptor® (delavirdine), Sustiva® (efavirenz), Viramune® (nevirapine, 11-cyclopropyl-4-methyl-5,11-dihydro-6H-dipyrido[3,2-b:2′,3′-e][1,4]diazepin-6-one), trisodium phosphonoformate, ammonium-21-tungstenato-9-antimonate, 1-O-D-ribofuranoxyl-1,2,4-triazole-3-carboxamide, Aganerase® (amprenavir), Reyataz® (atazanavir), Lexiva® (fosamprenavir), Crixivan® (indinavir), Viracept® (nelfinavir), Norvir® (ritonavir), Fortovase® or Invirase® (saquinavir), lasinavir (5(S)-(tert-butoxycarbonylamino)-4(S)-hydroxy-6-phenyl-2(R)(2,3,4-trimethoxyphenylmethyl)-hexanoyl-(L)-valyl-N-(2-metoxy-ethyl)-amide), adriamycin, KVX-478, VX-478, 141W94, AG-1343, KNI-272, U-96988, BILA-2011 BS (palinavir), polymannoacetate, Fuzeon® (enfuvirtide, T-20), Epzicom® (abacavir and lamivudine), Trizivir® (abacavir, lamivudine and zidovudine), Truvada® (emtricitabine and tenofir DF), Combivir® (lamivudine and zidovudine), Kaletra® (lopinavir and ritonavir), or a combination thereof.

In some aspects, the additional pharmaceutical agent is administered before the antigen binding polypeptide complex, antibody or antigen binding fragment thereof.

In some aspects, the additional pharmaceutical agent is administered after the antigen binding polypeptide complex, antibody or antigen binding fragment thereof.

In some aspects, the additional pharmaceutical agent is administered concurrently with the antigen binding polypeptide complex, antibody or antigen binding fragment thereof.

In some aspects, the additional pharmaceutical agent and the antigen binding polypeptide complex, antibody or antigen binding fragment thereof are administered in the same pharmaceutical composition.

In some aspects, the additional pharmaceutical agent and the antigen binding polypeptide complex, antibody or antigen binding fragment thereof are administered in different pharmaceutical compositions.

### EXAMPLES

The following examples are provided to further illustrate aspects of the disclosure, and are not meant to constrain the disclosure to any particular application or theory of operation.

### Example 1

### Design of Trispecific Antibodies

The trispecific antibody format follows a previously-described design configuration (DOI: 10.1126/science.aan8630). Briefly, a cross-over dual variable domain (CODV)-Ig bispecific antibody arm was heterodimerized with a conventional antibody arm using knob-into-hole (doi:10.1038/nbt0798-677) modifications in the CH3 domain of IgG1 Fc. Specifically, three classes of anti-SARS-CoV-2 neutralizing antibodies targeting spike protein receptor binding domains (RBDs) were used: B1-182.1 (class I), A19-46.1 (class II), and A19-61.1 (class III). These classes were tested in various combinations including each antibody Fv orientation and copy number to achieve bi- or tri-specificity. (As used herein, the term “Fv” represents the antigen binding domain of an antibody containing a variable heavy region and a variable light region.) These combinations were tested for best neutralizing breadth/potency and good manufacturability. In addition, “knob” (S354C/T366W), “hole” (Y349C/T366S/L368A/Y407V), and “LS” (M428L/N434S) (doi: 10.1038/nbt.1601) mutations were engineered into CH3 of the monospecific, bi- or tri-specific Fc region.

### Example 2

### Synthesis, Cloning and Expression of Multispecific Antibodies

After design of the amino acid sequences for each multispecific antibody, the four genes for each multispecific antibody were synthesized using human preferred codons (GenScript) and cloned into eukaryotic expression vectors. For each multispecific antibody expression, equal amounts of the 4 plasmid DNAs were transfected into Expi293 cells (Life Technology) using Expi293 transfection reagent (Life Technology) as previously reported (DOI: 10.1126/science.aan8630). The transfected cells were cultured in a shaker incubator at 120 rpm, 37° C., 9% C02 for 4-5 days.

Culture supernatants were harvested and filtered, the multispecific antibodies were purified over a Protein A (GE Health Science) column. Each multispecific antibody was eluted with IgG elution buffer (Pierce), immediately buffer exchanged with phosphate buffered saline (PBS) and concentrated using Centricon Plus-70 (Millipore Sigma) membrane filter unit. After concentration, each multispecific antibody was applied to a Superdex 200 16/600 size exclusion column (Cytiva) to remove aggregates and different species in the preparation. The fractions were then analyzed on reduced and non-reduced SDS-PAGE to identify the fractions that contained the monomeric multispecific antibody before combining them. The pooled fractions were then further concentrated, aliquoted and analyzed by SDS-PAGE as well as an analytical SEC column (Superdex 200 16/600) to verify purity.

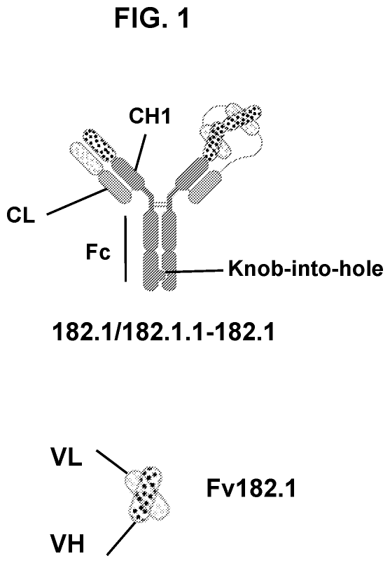

FIG. 1 shows an exemplary configuration of a monospecific, trivalent cross-over dual variable (CODV) immunoglobulin antibody made utilizing variable fragments (Fv) from B1-182.1 (Fv182.1, dotted). The Fc region contains a knob-into-hole modification to increase yield and correct association of heavy chains.

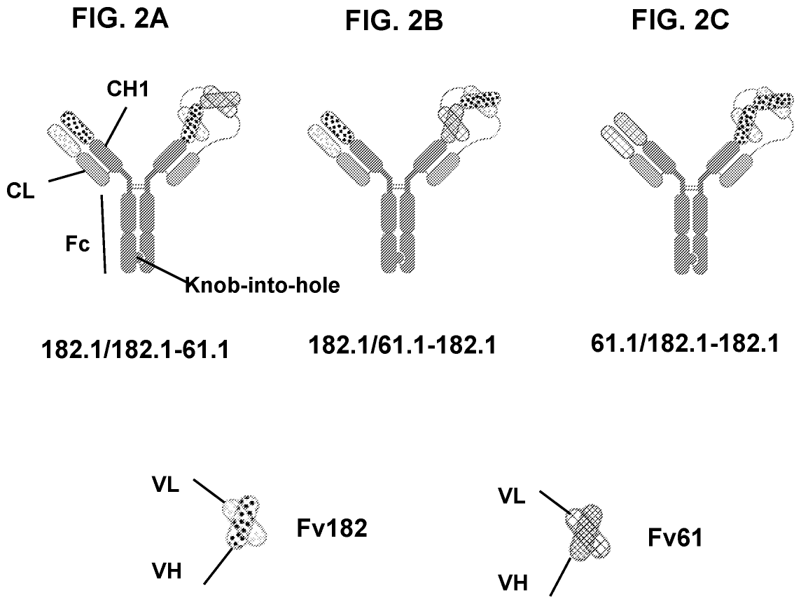

FIG. 2A-2C shows exemplary configurations of bispecific, trivalent CODV immunoglobulin antibodies made utilizing variable fragments (Fv) from B1-182.1 (Fv182.1, dotted) and A19-61.1 (Fv61.1, grid). The Fc region contains a knob-into-hole modification to increase yield and correct association of heavy chains.

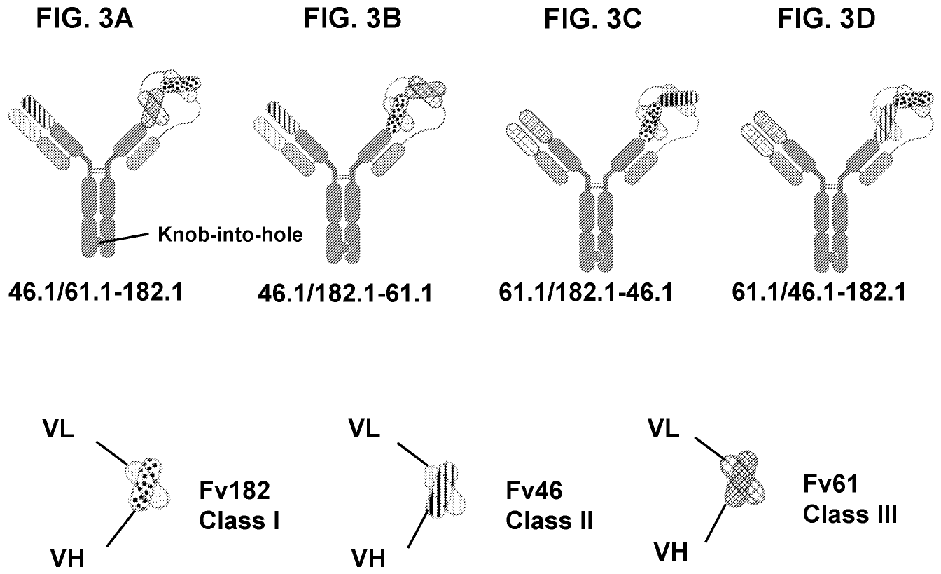

FIG. 3A-3D shows exemplary configurations of trispecific, trivalent CODV immunoglobulin antibodies made utilizing variable fragments (Fv) from B1-182.1 (“182.1” or “Fv182 Class I”, dotted), A19-46.1 (“46.1” or “Fv46 Class II”, striped) and A19-61.1 (“61.1” or “Fv61 Class III”, grid). The Fc region contains a knob-into-hole modification to increase yield and correct association of heavy chains.

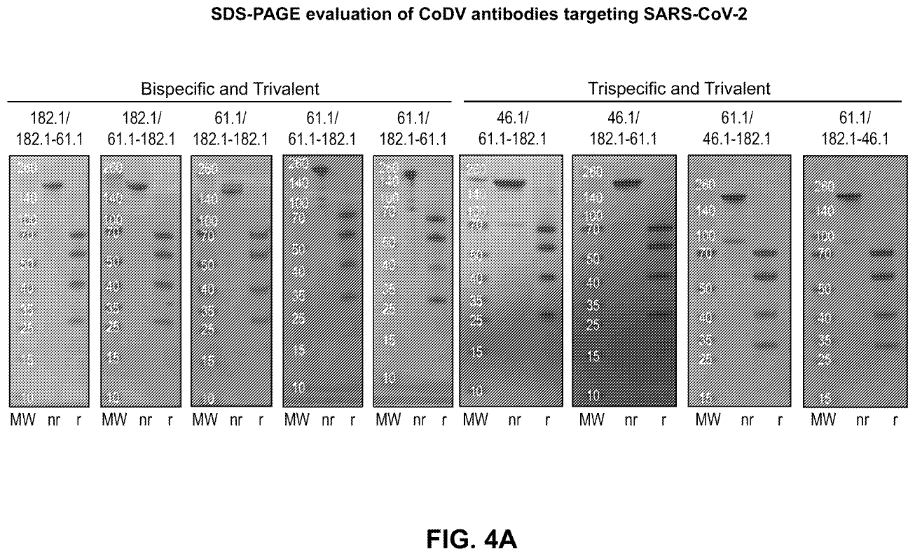

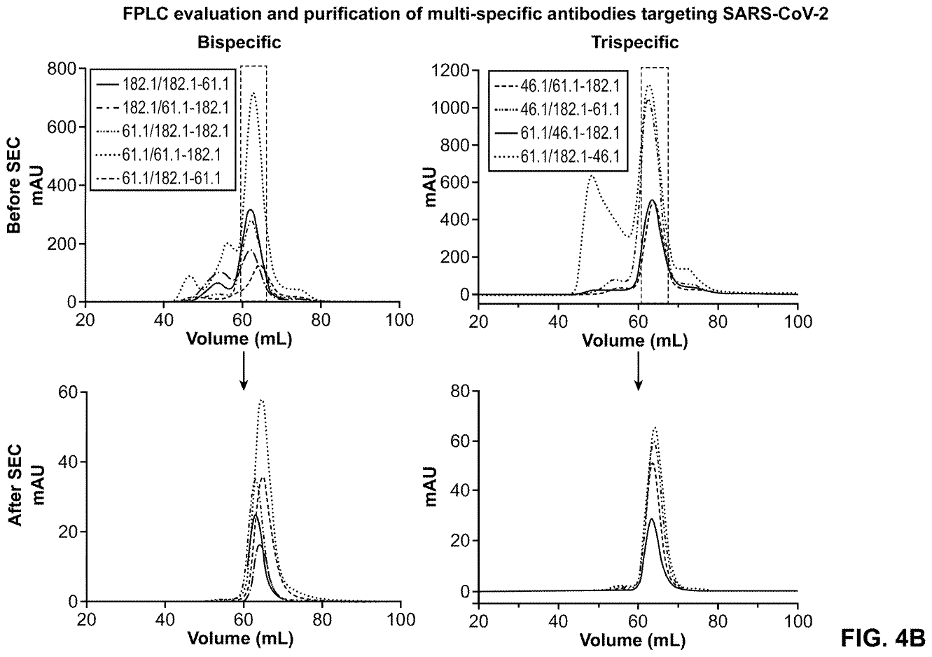

FIG. 4A shows purity of CODV immunoglobulin antibodies evaluated under non-reducing (nr) and reducing (r) conditions on a Coomassie SDS-PAGE gel (representative gels shown). Antibody sequences are shown below in Table 1.

TABLE 1
Sequences

| Antibody | SEQ ID NOS: |
|---|---|
| 182.1/182.1-61.1 | 113, 114, 119-122 |
| 182.1/61.1-182.1 | 93-96, 119-122 |
| 61.1/182.1-182.1 | 97-100, 115-118 |
| 61.1/61.1-182.1 | 97-100, 119-122 |
| 61.1/182.1-61.1 | 97-100, 113-114, 123, 124 |
| 46.1/61.1-182.1 | 87-89, 119-122 |
| 61.1/46.1-182.1 | 97-100, 83-86 |
| 61.1/182.1-46.1 | 97-100, 109-112 |

FIG. 4B shows CODV immunoglobulin bispecific and trispecific antibody traces shown before (top row) and after size exclusion chromatography (SEC) (bottom row). The dotted box indicates the fractions combined to make final preparations of the indicated multispecific antibodies. The bottom row shows analytic SEC traces for the purified multispecific antibodies. Shown are representative traces from production runs of antibodies. Antibody properties and yields are shown in Table 2.

TABLE 2
Biochemical Properties and Production Yields Following SEC
Purification For SARS-COV-2 CODV Antibodies

| | Molecular | | Extinction | |
|---|---|---|---|---|
| | Weight | Isoelectric | Coefficient. | Yield |
| ID | (kDa) | Point | (M⁻¹ cm⁻¹) | (mg/L) |
| 182.1/182.1-182.1 | 173.1 | 8.38 | 267120 | 14.5 |
| 182.1/182.1-61.1 | 173.4 | 8.39 | 271465 | 3.94 |
| 182.1/61.1-182.1 | 173.4 | 8.39 | 271465 | 2.6 |
| 61.1/182.1-182.1 | 173.4 | 8.39 | 271465 | 4.37 |
| 61.1/61.1-182.1 | 173.7 | 7.81 | 275810 | 1.6 |
| 61.1/182.1-61.1 | 173.7 | 7.81 | 275810 | 9.6 |
| 46.1/61.1-182.1 | 173.7 | 8.32 | 280280 | 9.24 |
| 46.1/182.1-61.1 | 173.7 | 8.32 | 280280 | 16.41 |
| 61.1/46.1-182.1 | 173.7 | 8.32 | 280280 | 7.94 |
| 61.1/182.1-46.1 | 173.7 | 8.32 | 280280 | 17.72 |

### Example 3

### Pseudovirus Neutralization Assay

The pseudotyped neutralization potency (IC50 and IC80) of multispecific antibodies described in Examples 1 and 2 was then determined. Specifically, S-containing lentiviral pseudovirions were produced by co-transfection of packaging plasmid pCMVdR8.2, transducing plasmid pHR′ CMV-Luc, a TMPRSS2 plasmid and S plasmids from SARS-CoV-2 variants into 293T cells using Lipofectamine 3000 transfection reagent (L3000-001, ThermoFisher Scientific, Asheville, NC). 293T-ACE2 cells (provided by Dr. Michael Farzan) were plated into 96-well white/black Isoplates (PerkinElmer, Waltham, MA) at 75,00 cells per well the day before infection of SARS CoV-2 pseudovirus. Serial dilutions of monoclonal antibodies (mAbs) were mixed with titrated pseudovirus, incubated for 45 minutes at 37° C. and added to cells in triplicate. Following 2 h of incubation, wells were replenished with 150 ml of fresh media. Cells were lysed 72 h later, and luciferase activity was measured with Microbeta (Perking Elmer). Percent neutralization and neutralization IC50 and IC80 values were calculated using GraphPad Prism 8.0.2.

Table 3 shows IC50 and IC80 values for neutralization of the SARS-CoV-2 original Wuhan strain (WT), SARS-CoV-2 D614G spike protein mutant (D614G), Alpha variant (B.1.1.7), Beta variant 1 (B.1.351.v1), Gamma variant (P.1), Delta variants (B.1.617.2 and AY.1), Kappa variant (B.1.617.1), Iota variant (B.1.526), Lambda variant (C.37), Mu variant (B.1.621), C.1.2 variant, and Omicron variants (BA1.1.529, BA.1, BA.2, BA.2.12.1 and BA.4/5). These results show that the antibodies tested potently inhibited many to all variants tested.

TABLE 3

| | WT | | D614G | | B.1.1.7 | | B.1.351.v1 | | P.1 | | B.1.617.2 | |
|---|---|---|---|---|---|---|---|---|---|---|---|---|
| Name | IC50 | IC80 | IC50 | IC80 | IC50 | IC80 | IC50 | IC80 | IC50 | IC80 | IC50 | IC80 |
| A23_58.1 | | | | | 0.0329 | 0.1041 | 0.1227 | 0.3389 | 0.0097 | 0.0437 | 0.0133 | 0.0358 |
| B1-182.1 | 0.0264 | 0.0692 | 0.0171 | 0.0359 | 0.0262 | 0.0844 | 0.0357 | 0.1390 | 0.0182 | 0.0429 | 0.0099 | 0.0254 |
| 58H_182L | | | | | 0.0174 | 0.0376 | 0.0305 | 0.0966 | 0.0116 | 0.0237 | 0.0104 | 0.0205 |
| A19-61.1 | 0.1725 | 0.4078 | 0.0550 | 0.1258 | 0.1747 | 0.3251 | 0.0686 | 0.1127 | 0.1352 | 0.2885 | 0.2090 | 0.2917 |
| A19-46.1 | 0.2633 | 0.7783 | 0.1379 | 0.3893 | 0.1899 | 0.4859 | 0.8074 | 2.0716 | 0.2600 | 0.7000 | >66.7 | >66.7 |
| 58H_182L/ | 0.0077 | 0.0223 | 0.0076 | 0.0204 | 0.0183 | 0.0398 | 0.0111 | 0.0339 | 0.003468 | 0.010518 | 0.003844 | 0.009797 |
| 58H_182L/ | | | | | | | | | | | | |
| 58H_182L | | | | | | | | | | | | |
| 182.1/182.1-61.1 | 0.0321 | 0.0966 | 0.0178 | 0.0447 | 0.0385 | 0.1148 | 0.0358 | 0.0806 | 0.019834 | 0.064068 | 0.011632 | 0.033888 |
| 182.1/61.1-182.1 | 0.0694 | 0.1891 | 0.0406 | 0.0812 | 0.0918 | 0.2084 | 0.0446 | 0.1080 | 0.044369 | 0.117965 | 0.014564 | 0.036509 |
| 61.1/182.1-182.1 | 0.2545 | 0.6804 | 0.0495 | 0.1236 | 0.2383 | 0.3381 | 0.1189 | 0.2217 | 0.214617 | 0.537649 | 0.068636 | 0.141241 |
| 61.1/61.1-182.1 | | | 0.0620 | 0.1181 | | | 0.0586 | 0.1268 | | | 0.1316 | 0.2455 |
| 61.1/182.1-61.1 | | | 0.0262 | 0.0583 | | | 0.0626 | 0.1369 | | | 0.0580 | 0.1217 |
| 46.1/61.1-182.1 | 0.2969 | 0.5228 | 0.0881 | 0.1538 | 0.2829 | 0.5068 | 0.1550 | 0.1989 | 0.1999 | 0.4556 | 0.0859 | 0.1828 |
| 46.1/182.1-61.1 | 0.1473 | 0.3613 | 0.0346 | 0.0769 | 0.1008 | 0.2644 | 0.0384 | 0.0816 | 0.0284 | 0.0887 | 0.2503 | 0.8762 |
| 61.1/46.1-182.1 | 0.0769 | 0.2880 | 0.0326 | 0.0764 | 0.0784 | 0.2296 | 0.0548 | 0.1262 | 0.0451 | 0.1553 | 0.1540 | 0.1955 |
| 61.1/182.1-46.1 | 0.3553 | 0.5566 | 0.0930 | 0.1539 | 0.2582 | 0.2992 | 0.1455 | 0.1799 | 0.2072 | 0.4977 | 0.3807 | 0.4716 |
| 58H_182L/ | | | | | 0.0412 | 0.1176 | 0.0582 | 0.1363 | 0.020744 | 0.082974 | 0.018507 | 0.056465 |
| 58H_182L-61.1 | | | | | | | | | | | | |
| 58H_182L/61.1- | | | | | 0.0967 | 0.2177 | 0.0635 | 0.1450 | 0.056899 | 0.106242 | 0.035644 | 0.062195 |
| 58H_182L | | | | | | | | | | | | |
| 46.1/61.1- | | | | | 0.3513 | 0.4160 | 0.2423 | 0.3083 | 0.2459 | 0.2713 | 0.1568 | 0.2862 |
| 58H_182L | | | | | | | | | | | | |
| 61.1/46.1- | | | | | 0.1005 | 0.3097 | 0.1059 | 0.1949 | 0.0455 | 0.1085 | 0.2301 | 0.2747 |
| 58H_182L | | | | | | | | | | | | |

| | B.1.617.1 | | B.1.526 | | C.37 | | B.1.621 | | C.1.2 | | AY.1 | |
|---|---|---|---|---|---|---|---|---|---|---|---|---|
| Name | IC50 | IC80 | IC50 | IC80 | IC50 | IC80 | IC50 | IC80 | IC50 | IC80 | IC50 | IC80 |
| A23_58.1 | | | | | | | 0.1240 | 0.2600 | 0.0287 | 0.0656 | | |
| B1-182.1 | 0.0261 | 0.0860 | 0.0639 | 0.1831 | 0.0049 | 0.0150 | 0.0695 | 0.2188 | 0.0549 | 0.1096 | 0.0080 | 0.0192 |
| 58H_182L | | | | | | | 0.0402 | 0.1081 | 0.0207 | 0.0348 | | |
| A19-61.1 | 0.0815 | 0.1732 | 0.0418 | 0.0836 | 0.1099 | 0.1865 | 0.2639 | 0.4296 | 8.4254 | | 0.0557 | 0.1207 |
| A19-46.1 | >66.7 | >66.7 | 0.8033 | 1.8820 | >66.7 | >66.7 | 1.3387 | 3.8125 | 11.6200 | 21.0215 | >66.7 | >66.7 |
| 58H_182L/ | | | | | | | 0.0201 | 0.0581 | 0.0042 | 0.0098 | 0.0113 | 0.0247 |
| 58H_182L/ | | | | | | | | | | | | |
| 58H_182L | | | | | | | | | | | | |
| 182.1/182.1-61.1 | 0.0105 | 0.0311 | 0.0314 | 0.0711 | 0.0083 | 0.0144 | 0.0710 | 0.1460 | 0.0381 | 0.0866 | 0.0049 | 0.0138 |
| 182.1/61.1-182.1 | 0.0142 | 0.0403 | 0.0446 | 0.0660 | 0.0103 | 0.0203 | 0.1122 | 0.2471 | 0.0794 | 0.0944 | 0.0099 | 0.0154 |
| 61.1/182.1-182.1 | 0.0405 | 0.0903 | 0.1146 | 0.1858 | 0.0371 | 0.0629 | 0.1947 | 0.3113 | | | 0.0412 | 0.0594 |
| 61.1/61.1-182.1 | | | | | | | | | | | | |
| 61.1/182.1-61.1 | | | | | | | | | | | | |
| 46.1/61.1-182.1 | 0.1047 | 0.2997 | 0.0694 | 0.1060 | 0.0902 | 0.2529 | 0.2226 | 0.3168 | 0.1148 | 0.2665 | 0.0441 | 0.1220 |
| 46.1/182.1-61.1 | 0.4675 | 0.9759 | 0.0362 | 0.0670 | 0.2156 | 0.6651 | 0.0389 | 0.1157 | | | 0.0882 | 0.4172 |
| 61.1/46.1-182.1 | 0.0753 | 0.1903 | 0.0525 | 0.0854 | 0.0690 | 0.1149 | 0.1559 | 0.3001 | 0.0654 | 0.1943 | 0.0438 | 0.0845 |
| 61.1/182.1-46.1 | 0.2051 | 0.3456 | 0.0808 | 0.1168 | 0.2133 | 0.2801 | 0.2716 | 0.4677 | | | 0.2629 | 0.2880 |
| 58H_182L/ | | | | | | | 0.0966 | 0.1854 | 0.0512 | 0.0650 | | |
| 58H_182L-61.1 | | | | | | | | | | | | |
| 58H_182L/61.1- | | | | | | | 0.2409 | 0.2656 | 0.0071 | 0.0194 | | |
| 58H_182L | | | | | | | | | | | | |
| 46.1/61.1- | | | | | | | 0.2862 | 0.3135 | 0.2185 | 0.4543 | | |
| 58H_182L | | | | | | | | | | | | |
| 61.1/46.1- | | | | | | | 0.2698 | 0.2858 | 0.0681 | 0.0878 | | |
| 58H_182L | | | | | | | | | | | | |

| | BA1.1.529 | | BA.1 | | BA.2 | | BA.2.12.1 | | BA.4/5 | |
|---|---|---|---|---|---|---|---|---|---|---|
| Name | IC50 | IC80 | IC50 | IC80 | IC50 | IC80 | IC50 | IC80 | IC50 | IC80 |
| A23_58.1 | | | 0.8529 | 4.2362 | 1.7240 | 6.1249 | 0.6471 | 4.9635 | >66.7 | >66.7 |
| B1-182.1 | 2.5540 | 6.4690 | 1.0322 | 4.4388 | 2.1151 | 8.5007 | 0.7393 | 5.9249 | >66.7 | >66.7 |
| 58H_182L | | | 0.3638 | 1.3930 | 0.6631 | 2.8936 | 0.8298 | 3.2735 | >66.7 | >66.7 |
| A19-61.1 | >66.7 | >66.7 | >66.7 | >66.7 | 0.0607 | 0.1699 | 0.1104 | 0.2980 | 0.1282 | 0.3220 |
| A19-46.1 | 1.8360 | 2.8400 | 0.9580 | 2.2810 | 0.6896 | 2.2272 | >66.7 | >66.7 | >66.7 | >66.7 |
| 58H_182L/ | | | 3.9134 | 24.2954 | 2.3442 | 25.0890 | 4.2697 | >66.7 | >66.7 | >66.7 |
| 58H_182L/ | | | | | | | | | | |
| 58H_182L | | | | | | | | | | |
| 182.1/182.1-61.1 | 47.2166 | | 27.2608 | 38.1161 | 0.0632 | 0.1724 | 0.1446 | 0.5118 | 10.0891 | 19.6789 |
| 182.1/61.1-182.1 | 1.2104 | | 1.0695 | 8.1047 | 0.0160 | 0.0626 | 0.0381 | 0.0788 | 2.4939 | 6.9402 |
| 61.1/61.1-182.1 | >66.7 | >66.7 | 0.0677 | 0.1171 | 0.0698 | 0.2894 | 0.0973 | 0.2350 | >66.7 | >66.7 |
| 61.1/182.1-61.1 | >66.7 | >66.7 | 0.0415 | 0.0962 | 0.0507 | 0.1275 | 0.0427 | 0.2009 | >66.7 | >66.7 |
| 61.1/182.1-182.1 | >66.7 | | >66.7 | >66.7 | 0.0859 | 0.2015 | 0.3427 | 0.6587 | 1.8459 | 6.0570 |
| 46.1/61.1-182.1 | 0.8634 | | 0.7366 | 1.8668 | 0.0400 | 0.0900 | 0.3527 | 1.1248 | 11.2895 | 24.6235 |
| 46.1/182.1-61.1 | 1.8580 | | 1.4133 | 3.9563 | 0.0448 | 0.1123 | 1.3441 | 2.4544 | 15.4292 | 36.9370 |
| 61.1/46.1-182.1 | 0.1666 | | 0.1982 | 0.4505 | 0.0255 | 0.0683 | 0.0488 | 0.1064 | 1.2141 | 2.3808 |
| 61.1/182.1-46.1 | 8.3726 | | 5.0902 | 23.0975 | 0.0195 | 0.0663 | 0.3086 | 0.6151 | 1.9607 | 4.4528 |
| 58H_182L/ | | | 22.1760 | 48.0214 | 0.1301 | 0.4851 | 0.6181 | 1.2993 | 16.1758 | 47.7654 |
| 58H_182L-61.1 | | | | | | | | | | |
| 58H_182L/61.1- | | | 2.7327 | 11.6180 | 0.0300 | 0.0960 | 0.0532 | 0.1416 | 2.3381 | 6.5572 |
| 58H_182L | | | | | | | | | | |
| 46.1/61.1- | | | 1.3652 | 3.0719 | 0.0283 | 0.1451 | 0.3664 | 0.7078 | 8.1859 | 19.2642 |
| 58H_182L | | | | | | | | | | |
| 61.1/46.1- | | | 0.2016 | 0.6393 | 0.0206 | 0.1003 | 0.1193 | 0.2270 | 2.1978 | 6.3612 |
| 58H_182L | | | | | | | | | | |

### Example 4

### Validation of Antibody Binding to RBD Mutation Proteins by ELISA and Neutralization

Binding of multispecific antibodies of Examples 1 and 2 to viral spike receptor-binding domain (RBD) mutations was then validated. The RBD mutations that were used contained one or more of the previously-identified knockout mutations for Fv182 (F486S), Fv46 (L452R) and Fv61 (K444E). For the validation assay, MaxiSorp™ Immuno Plates (Thermo Fisher Scientific) were coated with 1 g/mL of SARS-CoV-2 WA-1 RBD or RBD with single, double or triple mutations (F486S, E444K, L452R, F486S/E444K, F486S/L452R, and F486S/E444K/L452R) in PBS at 4° C. overnight. After standard washes and blocking, plates were incubated with serial dilutions of antibody for one hour at room temperature. Anti-human IgG Fc gamma-specific horseradish peroxidase conjugate (Jackson Laboratory) was used to detect binding of antibody to the RBD proteins. The plates were then washed and developed with 3,5,3′5′-tetramethylbenzidine (TMB) (KPL, Gaithersburg, MD). After stopping the reaction with 1N H₂SO₄ (Fisher), optical density (OD) at 450 nM was read with a SpectraMax® Plus microplate reader (Molecular Devices).

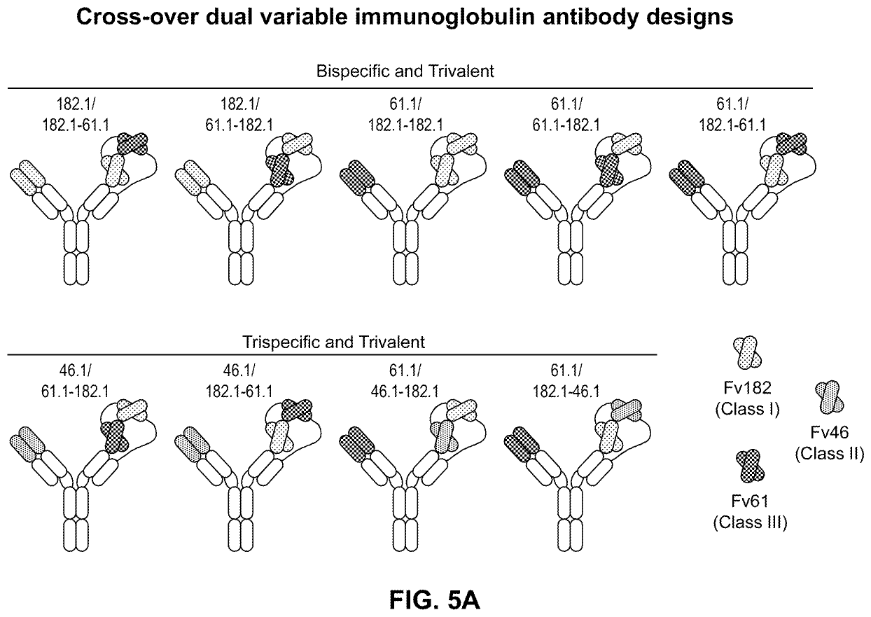

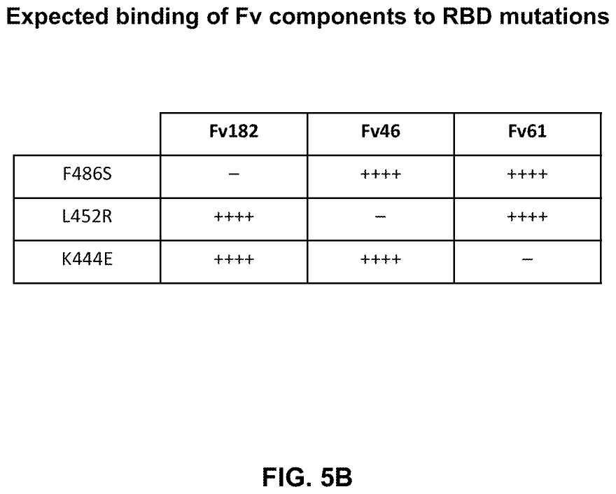

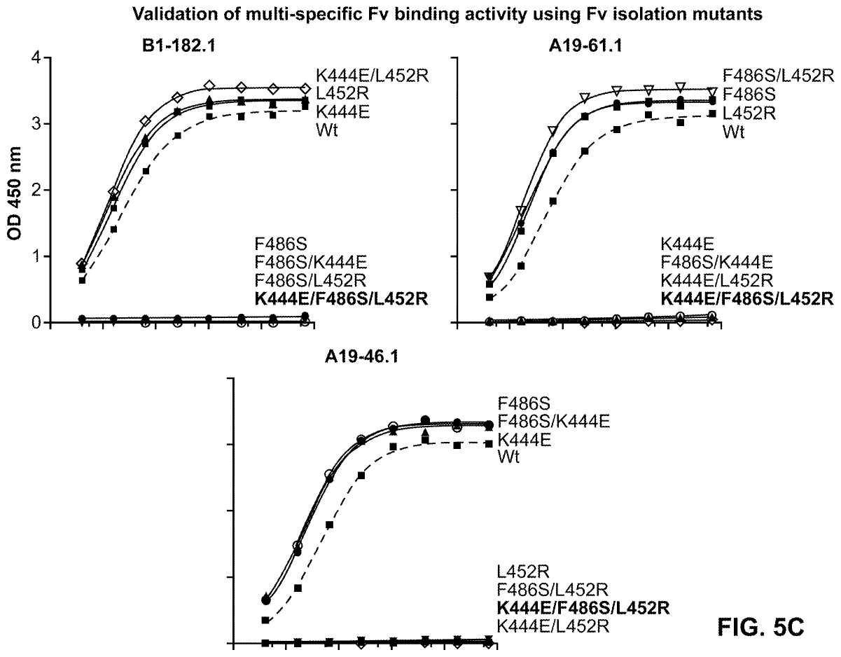

FIG. 5A shows cross-over the dual variable immunoglobulin antibody designs used in this example.

FIG. 5B shows the expected binding of Fv182.1 (“Fv182”), Fv46.1 (“Fv46”), and Fv61.1 (“Fv61”) to RBD proteins containing the indicated Fv isolating mutations that were selected to knockout binding a single component Fv, while leaving binding of the remain Fv components unchanged.

FIG. 5C-5E shows validation of multispecific Fv binding activity using Fv isolating mutations in RBD and enzyme-linked immunosorbent assay (ELISA). Shown is ELISA binding for parental component antibody controls (FIG. 5C, B1-182.1, A19-46.1, and A19-61.1), bispecific antibodies (FIG. 5D, 182.1/182.1-61.1, 182.1/61.1-182.1, 61.1/182.1-182.1, 61.1/61.1-182.1 and 61.1/182.1-61.1), and trispecific antibodies (FIG. 5E, 46.1/61.1-182.1, 46.1/182.1-61.1, 61.1/46.1-182.1 and 61.1/182.1-46.1) against WA-1 RBD (wt) or RBD proteins containing one or more Fv-specific mutations shown and chosen to interrogate binding of one Fv component at a time in each multispecific.

These results show that the candidate multispecific antibodies had the expected binding properties. For example, F486S/K444E showed binding to the Fv46 component of the trispecific antibodies because it does not allow binding of Fv182 and Fv61. And, Fv domains in each multispecific antibody recognized the unmutated epitopes similarly to wild type RBD.

For further validation, an assay was performed to test the ability of each antibody to neutralize spike protein point mutants that eliminated binding of one component and showed that each multispecific maintained neutralization. Table 4 shows the neutralization of candidate multispecific antibodies against D614G or mutants that target Fv182 (“B1-182.1 Escape”; F486S/D614G), Fv46 (“A19-46.1 Escape”; L452R/D614G) and Fv46 (“A19-46.1 Escape”; K444E/D614G).

TABLE 4

| | | | | B1-182.1 Escape | | A19-46.1 Escape | | A19-46.1 Escape | |
|---|---|---|---|---|---|---|---|---|---|
| | | D614G | | F486S/D614G | | L452R/D614G | | K444E/D614G | |
| | | IC₅₀ | IC₈₀ | IC₅₀ | IC₈₀ | IC₅₀ | IC₈₀ | IC₅₀ | IC₈₀ |
| | B1-182.1 | 15.7 | 34.9 | >67,000 | >67,000 | 25.0 | 75.8 | 11.1 | 29.9 |
| | A19-46.1 | 44.1 | 96.5 | 75.2 | 165.8 | 206.9 | 444.2 | >67,000 | >67,000 |
| | A19-61.1 | 113.3 | 339.2 | 132.9 | 390.0 | >67,000 | >67,000 | 472.1 | 1504 |
| Bispecific | 182.1/182.1-61.1 | 11.4 | 36.5 | 403.8 | 1320 | 24.1 | 112.6 | 7.0 | 56.1 |
| and | 182.1/61.1-182.1 | 15.8 | 46.8 | 354.1 | 789.6 | 38.5 | 65.2 | 10.1 | 35.8 |
| Trivalent | 61.1/182.1-182.1 | 67.4 | 128.4 | 320.4 | 1008 | 145.7 | 311.6 | 260.6 | 1090 |
| | 61.1/61.1-182.1 | n.t. | n.t. | n.t. | n.t. | n.t. | n.t. | n.t. | n.t. |
| | 61.1/182.1-61.1 | n.t. | n.t. | n.t. | n.t. | n.t. | n.t. | n.t. | n.t. |
| Trispecific | 46.1/61.1-182.1 | 48.7 | 119.2 | 112.1 | 243.2 | 201.2 | 602.8 | 189.1 | 487.4 |
| and | 46.1/182.1-61.1 | 31.7 | 62.9 | 80.0 | 270.8 | 278.7 | 909.9 | 46.9 | 140.5 |
| Trivalent | 61.1/46.1-182.1 | 30.8 | 48.4 | 68.6 | 163.9 | 232.8 | 934.0 | 42.0 | 139.9 |
| | 61.1/182.1-46.1 | 82.0 | 183.3 | 82.6 | 212.3 | 1010 | 2097 | 89.3 | 356.1 |

Taken together, these findings verified the Fvs within each candidate multispecific antibody functioned as expected.

### Example 5

### Identification of Potential Binding Modes of CODV to SARS-CoV-2 Spike Protein by Negative Stain-Electron Microscopy

Next, potential binding modes of CODV to SARS-CoV-2 spike protein were identified by negative stain-electron microscopy. For negative stain-electron microscopy, SARS-CoV-2 spike proteins were mixed with CODV or Fab at a molar ratio of 1:1.2, incubated at room temperature for 10 minutes, and diluted to a concentration of approximately 0.02 mg spike protein/mL with 10 mM HEPES, pH 7.4, 150 mM NaCl. To make a grid, 4.8 μL of the diluted sample was placed on a freshly glow-discharged carbon-coated copper grid for 15 seconds. The drop on the grid was then wicked away with filter paper, and the grid was washed and wicked three times. The same volume of 0.75% uranyl formate was added to the grid to negatively stain protein molecules adsorbed to the carbon and immediately wicked away. After staining three times, the grid was allowed to air-dry. Datasets were collected using a Thermo Scientific™ Talos™ F200C transmission electron microscope equipped with a Ceta camera at 200 kV. The nominal magnification was 57,000×, corresponding to a pixel size of 2.53 Å, and the defocus was set at −2 μm. Data was collected automatically using EPU. Single particle analysis was performed using CryoSPARC 3.0.

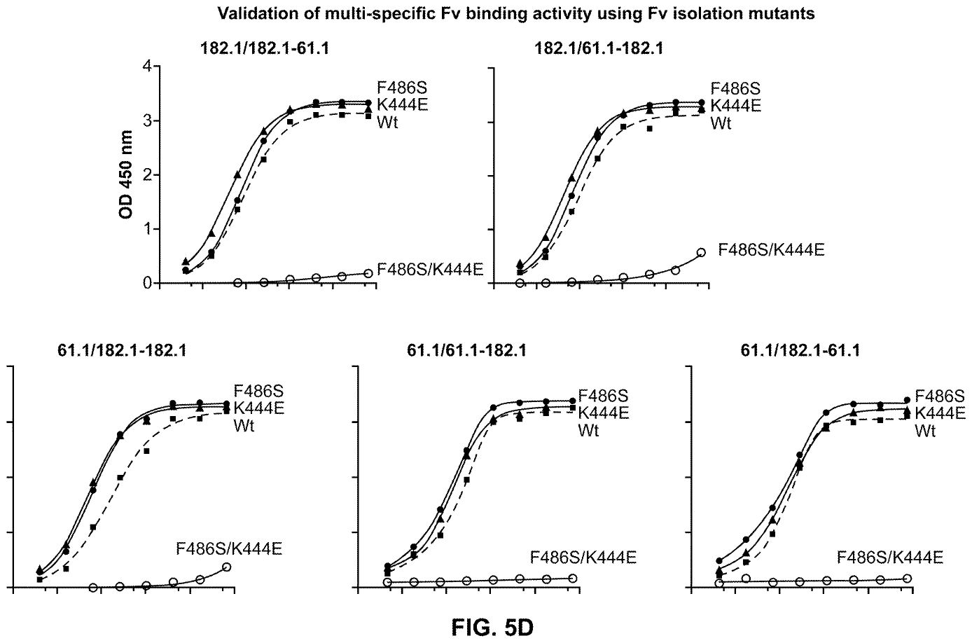

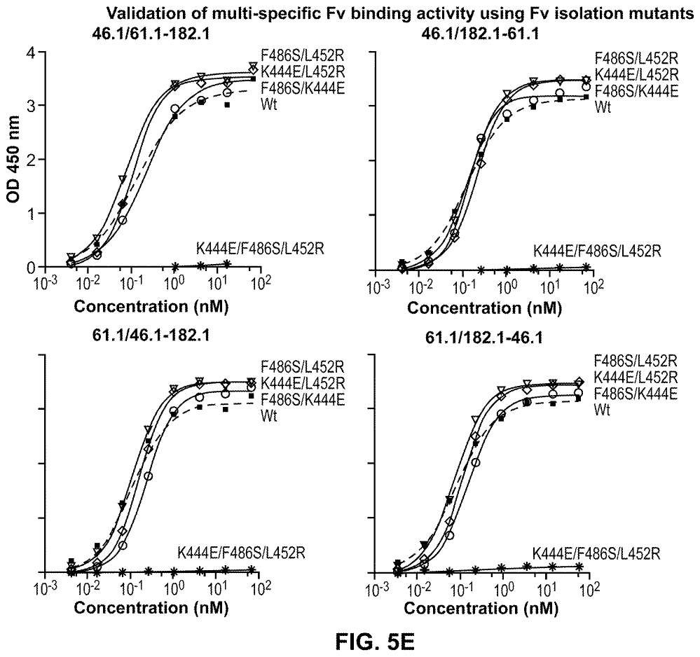

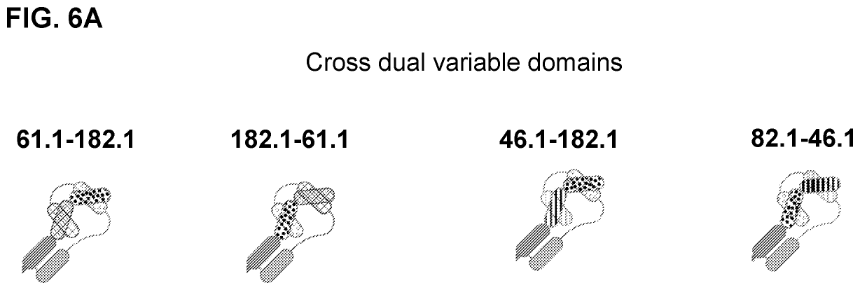

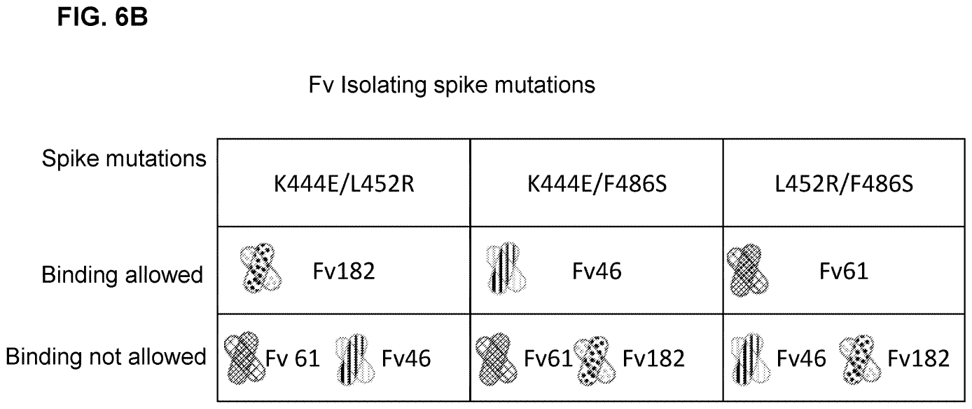

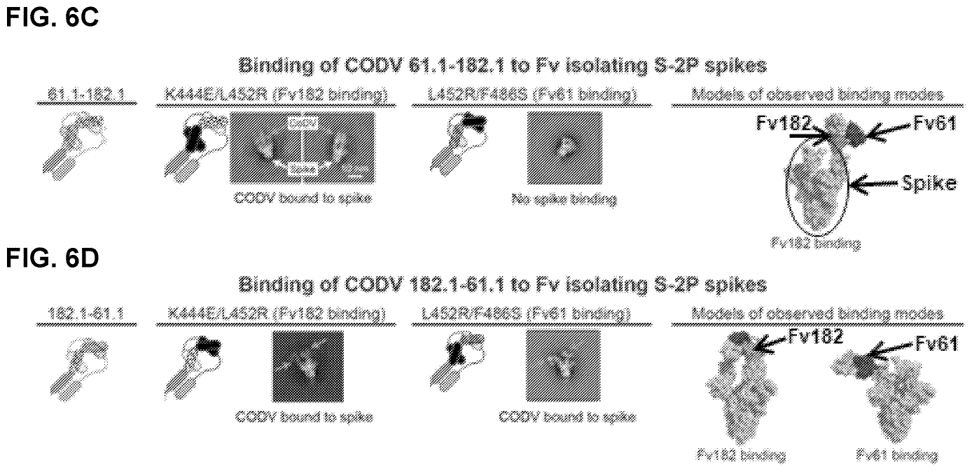

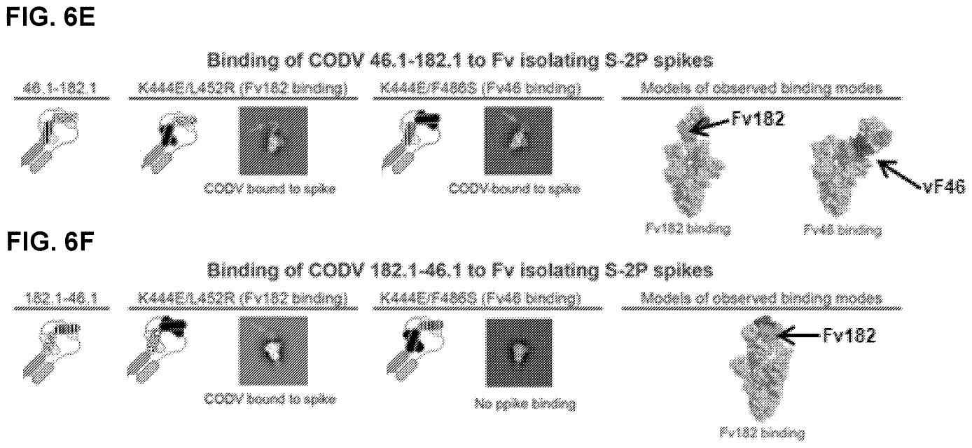

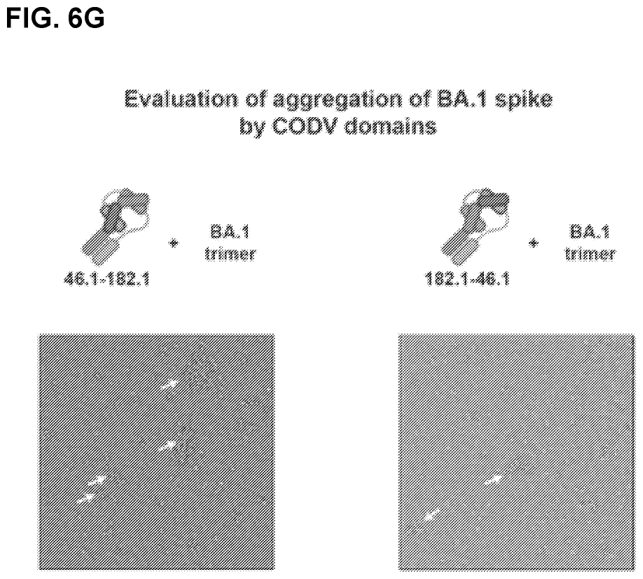

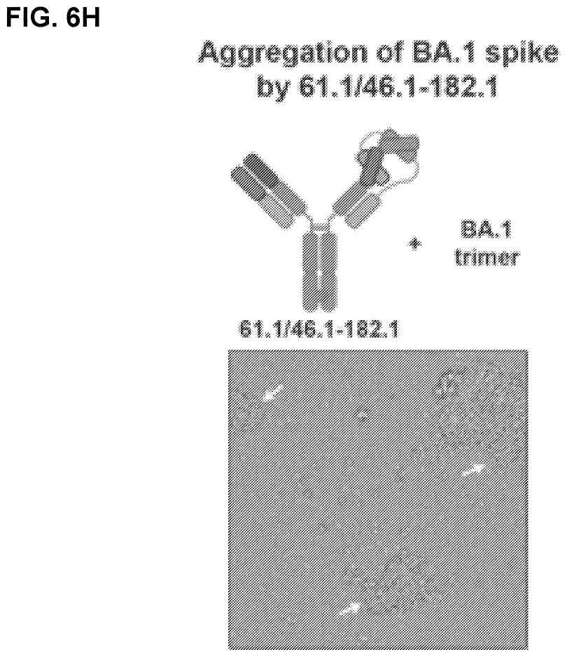

FIG. 6A shows a schematic illustration of arrangement of antibody variable domains in different CODVs. The domains are dotted, striped, grid, and gray for Fv182.1 (182.1), Fv46.1 (46.1), Fv61.1 (61.1) and constant domains, respectively.

FIG. 6B shows combinations of RBD mutations on the SARS-CoV-2 spike protein designed to distinguish different binding modes of CODV.

FIG. 6C is a schematic of CODV 61.1-182.1 (left) and binding of the CODV 61.1-182.1 to spike protein mutants (middle left and right). For clarity, the Fv domain that is not able to bind to the mutant is colored black. CODV binding to K444E/L452R mutant was observed, indicating CODV 61.1-182.1 can bind to spike protein through Fv182 domain (middle left). However, no CODV was observed to bind the L452R/F486S mutant (middle right), suggesting that the CODV 61.1-182.1 cannot bind through the Fv61 domain in the trimer context. A model of the CODV 61.1-182.1 bound to the SARS-CoV-2 spike protein is shown in the allowed Fv182-binding mode (right). For clarity, only one CODV is shown to bind to the spike protein in RBD-up conformation. The white scale bar represents 10 nm.

FIG. 6D is a schematic of CODV 182.1-61.1 (left) and binding of the CODV 182.1-61.1 to spike protein mutants (middle left and right). For clarity, the Fv domain that is not able to bind to the mutant is colored black. CODV was observed to bind both K444E/L452R and L452R/F486S mutants, indicating the CODV 182.1-61.1 can bind to spike protein through either Fv182 domain (middle left) or Fv61 domain (middle right). Models of the CODV 182.1-61.1 bound to the SARS-CoV-2 spike protein are shown in both Fv182.1-binding and Fv61.1-binding modes (right). The scale is the same as in FIG. 6C.

FIG. 6E is a schematic of CODV 46.1-182.1 (left) and binding of the CODV 46.1-182.1 to spike protein mutants (middle left and right). For clarity, the Fv domain that is not able to bind to the mutant is colored black. CODV was observed to bind both K444E/L452R and K444E/F486S mutants, indicating the CODV 46.1-182.1 can bind to the spike protein through both Fv182 and Fv46 domains (middle left and right). Models of the CODV 46.1-182.1 bound to the SARS-CoV-2 spike protein are shown to bind through Fv182 and Fv46-binding modes (right). The scale is the same as in FIG. 6C.

FIG. 6F is a schematic of CODV 182.1-46.1 (left) and binding of the CODV 182.1-46.1 to spike protein mutants (middle left and right). For clarity, the Fv domain that is not able to bind to the mutant is colored black. CODV was observed to bind K444E/L452R, indicating the CODV 182.1-46.1 can bind to the spike protein through the Fv182.1 domain (middle left). However, no CODV was observed to bind the K444E/F486S mutant (middle right), suggesting that the CODV 182.1-46.1 cannot bind through the Fv46 domain in the trimer context. Model of the CODV 182.1-46.1 bound to the SARS-CoV-2 spike protein is shown to bind through the allowed Fv182 domain (right). The scale is the same as in FIG. 6C.

FIG. 6G shows CODV 46.1-182.1- and 182.1-46.1-induced aggregation of the Omicron spike protein. NSEM micrographs revealed that under conditions when CODV 182.1-46.1 only formed small complexes with BA.1 spike trimers, CODV 46.1-182.1 formed large aggregates. This suggests that CODV 46.1-182.1 can efficiently promote inter-spike crosslinking.

While the monovalent 61.1 arm of 61.1/46.1-182.1 is prevented from binding to the Omicron BA.1 spike due to mutations, it is possible that its presence in the full immunoglobulin might impact the ability of the CODV 46.1-182.1 arm to aggregate spikes. The spike-aggregating potential of 61.1/46.1-182.1 using NSEM and noted large amounts of aggregation (FIG. 611). Taken together, these results illustrate how in the context of spike trimers, both the location of epitopes within RBD and CODV position can impact binding, aggregative potential and neutralization. Specifically, Fvs with an angle of approach vertical to the trimer apex such as Fv182 are likely to have greater flexibility for positioning with trivalent mAb designs, likely due to freedom from steric constraints, and consistent with higher potency neutralization by this Fv. In contrast, Fv46 has a lateral or angled approach suggesting that antibodies that bind with this angle of approach may be subject to steric constraints that are revealed by position-dependence of the Fv for optimal engagement and neutralization.

### Example 6

### SARS-CoV-2 Escape Potential by Single Antibodies, Combinations and Multispecific Antibodies

The escape potential of the candidate multispecific antibodies was assessed using an rcVSV antibody escape assay. Selection of virus escape variants was conducted as previously described by Wang et al., Science, 373:1-15 (2021). Briefly, an equal volume of clonal population of replication competent vesicular stomatitis virus (rcVSV) with its native glycoprotein replaced by the Wuhan-1 spike protein (rcVSV SARS CoV-2) at an MOI of 0.01 was mixed with serial dilutions of antibodies (5-fold) in cell media to give the desired final antibody concentration. Antibody cocktails were mixed at equal ratios. Virus:antibody mixtures were incubated at 37° C. for 1 hour prior to being added to Vero E6 cells. Virus replication was assessed 72 hours after infection in the presence of selected antibodies. Supernatants from well with the highest concentration of antibody which showed evidence of viral replication (>20% cytopathic effect) was passaged into the subsequent rounds of selection. Infection, monitoring, and collection of supernatants was performed as in the initial round.

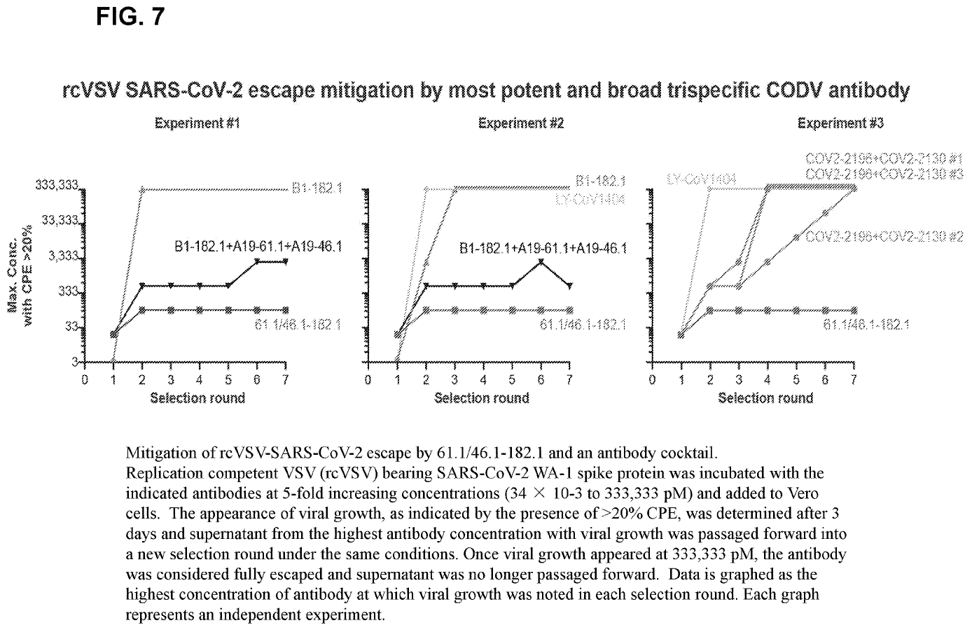

FIG. 7 shows SARS-CoV-2 escape potential by single antibodies, combinations of single antibodies, and multispecific antibodies. Shown for each antibody is the maximum concentration with >20% CPE that was passaged forward in each round of selection. Once significant viral growth appeared at 333,333 μM, supernatant was no longer passaged forward, and generation of escape variants was considered fixed.

These results show that the candidate multispecific antibodies mitigated immune escape observed with single antibodies and combinations of single antibodies.

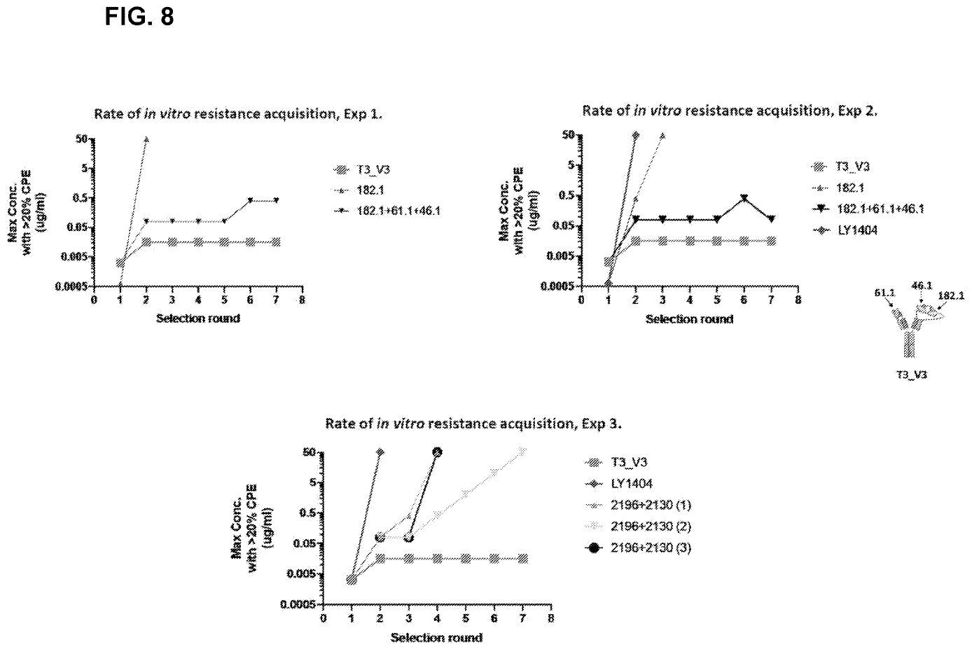

In addition, FIG. 8 shows results from three independent experiments showing SARS-CoV-2 escape potential of T3_V3 (having 61.1, 46.1 and 182.1 binding regions as indicated in FIG. 8), compared to an antibody having binding regions for 182.1, 61.1 and 46.1 (182.1+61.1+46.1); an antibody having binding regions for 182.1 (182.1); bebtelovimab (LY1404); and an AZD7442 cocktail (2196+2130). T3_V3 was better at preventing the acquisition of resistance than 182.1, LY1404 and AZD7442. In addition, the maximum concentration at which there was a loss of neutralization was 0.016 g/mL for T3_V3 in all three experiments.

### Example 7

### E8 mAb Glycosylation Mutations

N62Q and N62S mutations were made in the E8 heavy chain variable domain (VH) sequence, which remove a high mannose glycosylate residue or moiety. The specific sequences prepared are listed in Table 5.

TABLE 5

| SEQ ID NO: | Description |
|---|---|
| 175 | E8 VH wild type (N62) - peptide |
| 350 | E8 VH wild type (N62) - DNA |
| 351 | E8 VH N62Q - peptide |
| 352 | E8 VH N62Q - DNA |
| 353 | E8 VH N62S - peptide |
| 354 | E8 VH N62S - DNA |
| 357 | E8 VH N62Q in expression plasmid - peptide |
| 358 | E8 VH N62Q in expression plasmid - DNA |
| 359 | E8 VH N62S in expression plasmid - peptide |
| 360 | E8 VH N62S in expression plasmid - DNA |

The E8 VL sequence of SEQ ID NO:171 was used for preparing the antibodies (corresponding to SEQ ID NO:355 (E8 VL in expression vector—peptide) and SEQ ID NO:356 (E8 VL in expression plasmid—DNA)).

Human FcRn transgenic mice (C57BL/6, B6.mFcRn−/− hFcRn Tg32 line from The Jackson Laboratory) were used to assess the pharmacokinetics of the antibodies. Each animal was infused intravenously with 5 mg antibody/kg of body weight. Whole blood samples were collected at days 1, 2, 5, 7, 9, 14, 21, 28, and 35. Serum was separated by centrifugation. Serum antibody levels were measured by ELISA. All mice were bred and maintained under pathogen-free conditions at an American Association for Assessment and Accreditation of Laboratory Animal Care International-accredited animal facility at the National Institute of Allergy and Infectious Diseases and housed in accordance with the procedures outlined in the Guide for the Care and Use of Laboratory Animals. All mice were between 6 and 13 weeks of age.

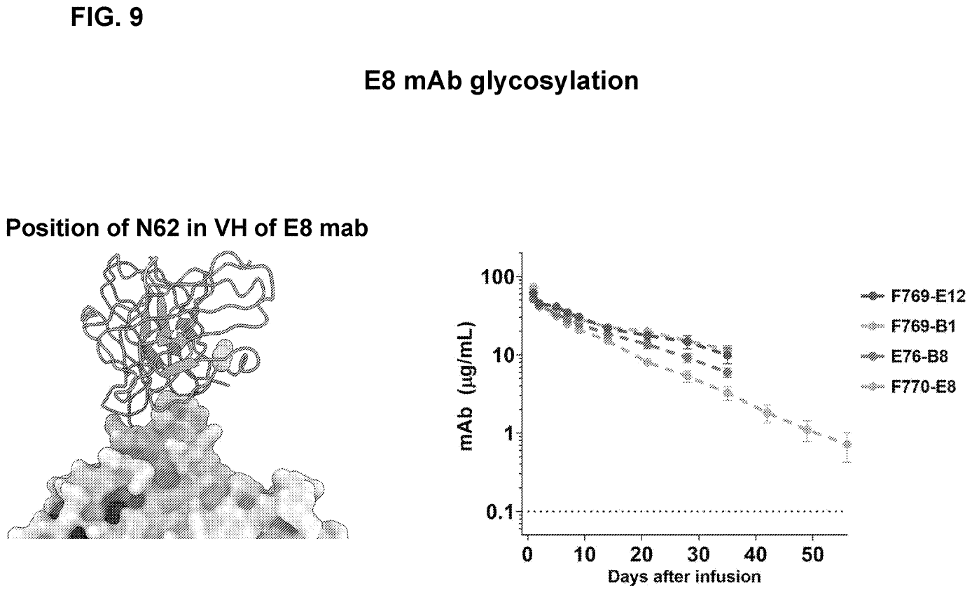

FIG. 9 shows the resulting serum concentration of E8 glycosylation mutant antibodies in g/mL.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. An antigen binding polypeptide complex comprising a first polypeptide, a second polypeptide, a third polypeptide, and a fourth polypeptide;
wherein the first polypeptide has a structure represented by:
VL1-L1-CL;
wherein the second polypeptide has a structure represented by:
VH1-L2-CH1-L3-Fc;
wherein the third polypeptide has a structure represented by:
VH2-L4-VH3-L5-CH1-L6-Fc;
wherein the fourth polypeptide has a structure represented by:
VL3-L7-VL2-L8-CL;
wherein:
VL1 is a first immunoglobulin light chain variable region that specifically binds to a SARS-CoV-2 protein;
VL2 is a second immunoglobulin light chain variable region that specifically binds to a SARS-CoV-2 protein;
VL3 is a third immunoglobulin light chain variable region that specifically binds to a SARS-CoV-2 protein;
VH1 is a first immunoglobulin heavy chain variable region that specifically binds to a SARS-CoV-2 protein;
VH2 is a second immunoglobulin heavy chain variable region that specifically binds to a SARS-CoV-2 protein;
VH3 is a third immunoglobulin heavy chain variable region that specifically binds to a SARS-CoV-2 protein;
Fc is a region comprising an immunoglobulin heavy chain constant region 2 (CH2), an immunoglobulin heavy chain constant region 3 (CH3), and optionally, an immunoglobulin hinge;
CH1 is an immunoglobulin heavy chain constant region 1;
CL is an immunoglobulin light chain constant region; and
L1-L8 are amino acid linkers.

3. The antigen binding polypeptide complex of claim 4, wherein VH1 comprises the same heavy chain variable region as VH3, and VL1 comprises the same light chain variable region as VL3.

4. The antigen binding polypeptide complex of claim 4, wherein VH1 comprises the same heavy chain variable region as VH2, and VL1 comprises the same light chain variable region as VL2.

5. The antigen binding polypeptide complex of claim 4, wherein VH2 comprises the same heavy chain variable region as VH3, and VL2 comprises the same light chain variable region as VL3.

6-27. (canceled)

28. The antigen binding polypeptide complex of claim 2, wherein linkers L1-L8 each independently have a length of from 0 amino acids to about 50 amino acids.

29-31. (canceled)

32. The antigen binding polypeptide complex of claim 2, wherein the Fc region comprises at least one knob-into-hole modification or Fc effector function knockout mutation.

33. The antigen binding polypeptide complex of claim 32, wherein the knob-into-hole modification comprises:
(i) knob substitutions of S354C and T366W and hole substitutions of Y349C, T366S, L368A and Y407V;
(ii) hole substitutions of M428L and N434S or N434A;
(iii) hole substitutions of M252Y, S254T and T256E; or
(iv) a combination thereof,
based on the EU numbering scheme.

34. (canceled)

35. The antigen binding polypeptide complex claim 2 comprising a detectable label.

36. The antigen binding polypeptide complex of claim 35, wherein the detectable label is a radioactive label, chemiluminescent label, fluorescent label, enzyme, peptide tag, or a combination thereof.

37-38. (canceled)

39. The antigen binding polypeptide complex of claim 2 that specifically binds to a SARS-CoV-2 protein with an equilibrium dissociation constant (K_{D}) of from about 10 μM to about 1 μM.

40. The antigen binding polypeptide complex of claim 2, wherein the SARS-CoV-2 virus is the original Wuhan strain (WA1), D614G spike protein mutant (D614G), Alpha variant (B.1.1.7), Beta variant 1 (B.1.351.v1), Gamma variant (P.1), Delta variant (B.1.617.2 or AY.1), Kappa variant (B.1.617.1), Iota variant (B.1.526), Lambda variant (C.37), Mu variant (B.1.621), C.1.2 variant, Epsilon variant (B.1.427), or Omicron variant (BA1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF.7 or CH.1.1), or a combination thereof.

41-49. (canceled)

50. A pharmaceutical composition comprising the antigen binding polypeptide complex of claim 2 and a pharmaceutically acceptable carrier.

51-62. (canceled)

63. A kit comprising the antigen binding polypeptide complex of claim 2.

64-70. (canceled)

71. A method of preventing or treating a SARS-CoV-2 viral infection in a subject in need thereof, comprising administering to the subject an effective amount of the antigen binding polypeptide complex of claim 2.

72. A method of preventing or treating coronavirus disease 2019 (COVID-19) in a subject in need thereof, comprising administering to the subject an effective amount of the antigen binding polypeptide complex of claim 2.

73. A method of diagnosing a subject as being infected with a SARS-CoV-2 virus or suspected of being infected with a SARS-CoV-2 virus, comprising (i) contacting a sample obtained from the subject with the antigen binding polypeptide complex of claim 2; (ii) detecting the presence or absence of a virus complex which contains the polypeptide complex, antibody or fragment and a SARS-CoV-2 virus, virion or fragment thereof, and (iii) diagnosing the subject as being infected with a SARS-CoV-2 virus or suspected of being infected with a SARS-CoV-2 virus when the presence of the virus complex is detected.

74. (canceled)

75. A method of diagnosing a subject as having COVID-19 or suspected of having COVID-19, comprising (i) contacting a sample obtained from the subject with the antigen binding polypeptide complex of claim 2; (ii) detecting the presence or absence of a virus complex which contains the polypeptide complex, antibody or fragment and a SARS-CoV-2 virus, virion or fragment thereof, and (iii) diagnosing the subject as having COVID-19 or suspected of having COVID-19 when the presence of the virus complex is detected.

76. (canceled)

77. The method of claim 73, wherein the sample is a nasal swab, tissue sample, saliva, plasma or blood.

78. The method of claim 73, wherein detecting the presence or absence of the virus complex comprises an enzyme linked immunosorbent assay (ELISA), immunospot assay, lateral flow assay, flow cytometry, immunohistochemistry or western blot.

79. The method of claim 71, wherein the SARS-CoV-2 virus is the original Wuhan strain (WA1), D614G spike protein mutant (D614G), Alpha variant (B.1.1.7), Beta variant 1 (B.1.351.v1), Gamma variant (P.1), Kappa variant (B.1.617.1), Delta variant (B.1.617.2 or AY.1), Epsilon variant (B.1.427), Iota variant (B.1.526), Lambda variant (C.37), Mu variant (B.1.621), C.1.2 variant, Omicron variant (BA1.1.529, BA.1, BA.2, BA.2.12.1, BA.4, BA.4/5, BA.5, BQ.1, BQ.1.1, BA.2.75, BA.2.75.2, BA.4.6, BQ.1.1, BJ.1, XBB, XBB1.5, BF. 7, or CH.1.1), or a combination thereof.

80-124. (canceled)
